(19) United States
(12) Patent Application Publication
(10) Pub. No.: US 2023/0285719 A1
(43) Pub. Date: Sep. 14, 2023

(54) DEVICES AND METHODS FOR VASCULAR NAVIGATION, ASSESSMENT, TREATMENT AND/OR DIAGNOSIS

(71) Applicant: Piccolo Medical, Inc., San Francisco, CA (US)

(72) Inventors: Alexey SALAMINI, San Francisco, CA (US); Jonathan SILBERSTEIN, San Francisco, CA (US); Augustus SHANAHAN, San Francisco, CA (US); Kevin MCDERMOTT, San Francisco, CA (US); John MCKENZIE, San Carlos, CA (US); Mark SPECTOR, San Francisco, CA (US); Daniel R. BURNETT, San Francisco, CA (US); Eric YU, San Francisco, CA (US); Mark HEALY, San Francisco, CA (US)

(73) Assignee: Piccolo Medical, Inc., San Francisco, CA (US)

(21) Appl. No.: 18/177,617

(22) Filed: Mar. 2, 2023

Related U.S. Application Data
(63) Continuation of application No. PCT/US2021/049050, filed on Sep. 3, 2021.

(51) Int. Cl. A61M 25/01; A61B 5/287

(52) U.S. Cl. CPC A61M 25/0102; A61B 5/287; A61M 25/01; A61M 2025/0166

(57) ABSTRACT

Devices and methods for vascular navigation, assessment, treatment and/or diagnosis are disclosed. The system generally comprises an elongate body sized for introduction and translation through a catheter lumen. One or more openings may be located at or in proximity to a distal end of the elongate body and a sensor is positioned at or in proximity to the distal end. The sensor is configured to measure at least one parameter of a mixture of a first fluid and a second fluid after the first fluid is emitted from the openings and into the second fluid when the distal end is advanced beyond a distal opening of the catheter lumen. A controller in communication with the sensor is configured to receive a signal indicative of the at least one parameter and is further configured to obtain a position of the sensor within a body of a subject based upon the signal.

### CROSS-REFERENCE TO RELATED APPLICATIONS

This application is a continuation of International Patent Application Number PCT/US2021/049050 filed Sep. 3, 2021, which claims the benefit of priority to U.S. Provisional Application No. 63/075,392 filed Sep. 8, 2020 which is incorporated herein by reference in its entirety.

### FIELD OF THE INVENTION

The present invention relates to devices and methods for vascular navigation, assessment, treatment and/or diagnosis.

### INCORPORATION BY REFERENCE

All publications and patent applications mentioned in this specification are herein incorporated by reference to the same extent as if each such individual publication or patent application were specifically and individually indicated to be so incorporated by reference.

### BACKGROUND OF THE INVENTION

A central vascular catheter (vascular catheter), also known as central line, central venous line or central venous access catheter, is a catheter placed into a large vein in the neck (internal jugular vein), chest (subclavian vein or axillary vein), arm or groin (femoral vein). It is primarily used to administer medication or fluids, obtain blood tests (such as central venous oxygen saturation), and measure central venous pressure.

A peripherally inserted central catheter (PICC or PIC line) is a form of vascular catheter that can be used for a prolonged period of time and/or for administration of substances. It is a catheter that enters the body through the skin (percutaneously) at a peripheral site, extends to the superior vena cava (a central venous trunk), and may remain in place for days or weeks.

Placing the catheter (PICC, central vascular catheter or related vascular catheter, referred to herein as “vascular catheter” or “catheter”) in the ideal location can be challenging. The catheter may be mistakenly inserted into an artery instead of a vein, or into the incorrect vein or incorrect venous branch or advanced too far or into/along a vessel wall. Ideally, the catheter tip is placed in the superior vena cava/cavo-atrial junction (SVC-CAJ or CAJ), or the lower one third of the superior vena cava.

Correct placement currently is determined by taking a physical measurement of the distance from the catheter entry point to the estimated location of the lower one third of the superior vena cava or CAJ. There are several challenges with current techniques. First, the catheter may enter into an artery instead of a vein. Second, a catheter may be advanced down the incorrect branch of the vein tree. The catheter may advance down an azygous vein, a thoracic vein, a jugular vein, or any number of additional veins on the branch. Third, a catheter may be advanced past the superior vena cava and into the heart or into the inferior vena cava. This can be a dangerous situation. Fourth, a catheter may advance up against, or embed in, a vessel wall which can prevent fluid delivery or fluid draw. Fifth, because the gold standard for catheter placement is essentially blind, placement verification needs to be confirmed with a chest x-ray which adds additional cost and time. Sixth, the estimated distance to the lower one third of the superior vena cava or CAJ may be inaccurate.

There is a need for a relatively easy and accurate way of navigating a vascular catheter by accurately identifying the location of the tip of the catheter as it is advanced to its targeted location.

### SUMMARY OF THE INVENTION

The present invention includes vascular catheter location and navigation devices and methods which determine the location of the tip of a vascular catheter using the introduction of a medium (or injectate) with a measurable parameter (temperature, light reflection, sound reflection, conductance, impedance, etc.) and sensing and measuring the measurable parameter as the catheter is advanced within a flowing fluid, such as blood flow in a blood vessel. Measurements of the parameter are tracked over time, recorded and analyzed. The value of the parameter and/or the shape of the parameter value vs. time curve may be used in the analysis. For example, curve amplitude, variability, pulsatility, phase, standard deviation, slope, etc. may be used in the analysis of catheter location.

Flow direction, characteristics, profiles, and types, with respect to the catheter and catheter tip can provide a vast array of information on catheter positioning during placement, after initial or subsequent placement, after the catheter has been in place for a period of time, and/or during catheter withdrawal.

Devices and methods disclosed herein can be used to inform the user of one or more of the following conditions: insertion, placement or advancement of the catheter into an artery rather than a vein; insertion, placement or advancement of the catheter into an undesired vein branch; placement or advancement of the catheter too near, into, or past the heart; or placement of the catheter tip up against, or embedded in, a wall of a vessel, or insufficient advancement of the catheter. Each of these scenarios is described in detail herein.

Blood flow characteristics and direction can help determine if the catheter is in an artery or a vein. In the case of a vein, the blood will generally be flowing more slowly toward the heart, while with the artery the blood will generally be flowing more quickly away from the heart. At least the blood flow direction and speed with respect to the catheter will be different depending on whether the catheter is in an artery or vein. Other flow parameters may also be different (turbulence, pulsatility, etc.). In addition, the flow characteristics of blood within a smaller branch of the blood vessel will be different than the flow characteristics in a larger vessel. For example, blood flow within a vein branch may completely or substantially stop where a catheter tip is totally or partially occluding the vein branch. In the case where the catheter tip is seated against a vessel wall, flow patterns around the catheter are different than when the catheter tip is in free flowing blood.

In the situation where the catheter tip passes into the superior vena cava, and passes near or into the heart's right atrium or right ventricle, the flow characteristics of the blood will change. For example, the blood flow may become more or less turbulent. More or less turbulence results in different flow characteristics, profiles, and flow types and can be detected by a variety of types of sensors.

These flow profile changes can be measured using devices and methods disclosed herein.

Devices disclosed herein may include a catheter, a guidewire, a stylet, a controller, communications, an infusion mechanism, a medium source, medium sensor or sensors etc.

Devices and methods disclosed herein utilize the introduction of a medium or injectate (saline, fluid, light, sound, etc.) which has a measurable parameter (temperature, conductivity, impedance, opacity, light reflectivity, sound reflectivity, density, viscosity, ability to absorb light, ability to absorb sound, amplitude, etc.) where the measurable parameter can be detected using a sensor (sensor, thermocouple, electrode, light sensor, sound sensor, microphone, etc.). By introducing a medium at or near the tip of the catheter and/or stylet, and measuring one or more parameters of the medium over time, and possibly over distance, flow parameters, such as flow direction, rate, volume and type, turbulent or laminar, can be determined. Based on these determinations, the user can identify whether the catheter tip is progressing to the desired position in the vasculature via the desired path. Vessels may be identified by type (vein vs. artery, vs heart etc.), size, shape, flow parameters may be assessed, etc.

The measurable parameter of the injectate medium is different from that of blood, either higher or lower. In some embodiments, the measurable parameter of the injectate medium or of blood may be zero or essentially zero. For example, where the parameter is conductivity, the injectate medium may be a zero conductivity fluid, such as distilled water or D5 W (dextrose 5% in water) similar.

The medium may be injected or introduced intermittently during all or part of catheter placement, continuously during all or part of catheter placement, periodically during all or part of catheter placement, continually during all or part of catheter placement, and/or at regular intervals during all or part of catheter placement. The medium may be introduced manually, or automatically via a controller, or automatically via an intravenous (IV) bag with or without an IV pump, or passively with an IV.

In some embodiments, the location of the sensors relative to where the medium, or injectate, is introduced is controlled. A sensor distal to the introduction of the medium may detect antegrade flow whereas a sensor proximal to the introduction of medium may detect retrograde flow.

Measurements of one or more medium parameters may be taken before, during and/or after medium introduction. For example, room temperature or other non-body temperature saline (or other fluid) may be injected through the catheter or stylet during placement. One or more sensors at or near the distal tip of the catheter/stylet can measure the temperature of the fluid immediately surrounding the sensor(s) over time as the device is advanced/moved. Based on blood flow characteristics, including direction, pulsatility and turbulence, the temperature profile over time will be different at different locations, resulting in a temperature (or parameter) profile or signature for different flow types and therefore different catheter/stylet tip location scenarios.

In embodiments where the device is used in fluid flow, for example in a blood vessel, the medium may be a fluid (first fluid) which has a measurable parameter that is different than that of the fluid within the vessel (second fluid, which may be blood). The sensors in any of the embodiments disclosed herein may be measuring the parameter of the mixture of the first fluid and the second fluid, over time and at different locations, to determine the location of the device. Note that in some embodiments, the medium parameter level may be negligible and may serve to dilute the parameter in the mixture of the first fluid and the second fluid. For example, where the parameter is electrical conductivity, the medium, or injectate, may be distilled water, or another injectate, which has negligible conductivity, where blood has a higher conductivity. In these embodiments, the sensors may be measuring the conductivity of the injected medium/blood mixture to determine device location.

Temperature sensors may include thermocouples or other temperature sensors, such as, fiber optic, resistive, bimetallic, thermometer, state-change, silicon diode, thermistors, optical temperature measurement (infrared or otherwise), mercury thermometers, manometers, etc. The sensor or sensors is/are in communication with a controller which records and/or analyzes the signal from the sensor(s). The communication between the sensor and the controller may be wired or wireless.

By placing a thermocouple, thermistor, or other temperature sensing device, or an array of temperature sensing devices on or through the catheter, one can determine the direction of flow of a room temperature fluid bolus that is injected into the blood stream. Since blood temperature is around 37 degrees C., a saline (or other) fluid bolus or fluid infusion with a temperature around 20-25 degrees C. or between 15 and 30 degrees C. or between 0 and 35 degrees C., or generally cooler than 37 degrees C. is distinguishable from body temperature and can be used to detect blood flow direction and characteristics, and therefore, device location.

Alternatively the fluid may be greater than body temperature, optimally about 40 C but ranging from about 39 C to 42 C or about 37 C to about 45 C.

In some embodiments, optical sensing can be used. Optical sensors can be used to detect the direction of flow by measuring the amount of dilution of blood with another fluid with different optical characteristics, such as saline.

Sonar or sound can alternatively be used as the parameter to detect blood flow direction, velocity and other blood flow characteristics. Sound waves may be produced by the controller and conveyed to the tip, or near the tip, of the catheter. A sound detector, or microphone, records the sound waves reflected back by the red blood cells or other components of blood. Saline may also be introduced to create a change in the sound waves detected.

Various mediums and/or parameters may be used in combination in some embodiments. For example, light (visible and/or not visible) and temperature may both be used. In addition, other sensors may be used to aid in locating the catheter, including electro cardiogram (ECG), ultrasound, Doppler, x-ray, etc. Pressure may also be used instead of, or in combination with these embodiments.

Other types of sensing may be used to detect various properties of blood, or a mixture of blood and an added element, such as fluid. For example, any of the following properties may be sensed using any of the sensing techniques disclosed herein, including sensing techniques below:

| Property | Sensing techniques |
|---|---|
| Concentration of RBCs | Optical flow sensors |
| Flow direction and speed | Reflection densitometry |
| Salinity | Interference refractometry |
| Pressure | Optical densitometry |
| Oxygen saturation | Refractive index |
| | In-fiber optic salinity sensing |
| | Diffuse reflectance spectroscopy |
| | Oxygen saturation |

Embodiments that incorporate more than one type of sensor may be used either in each situation (vein vs. artery, vessel branch, vessel wall, catheter in heart or past heart), or different sensors may be used in different situations. For example, pressure may be used to determine when the catheter tip is in the heart, where temperature may be used to determine whether the catheter is in an artery. Or, for example, ECG can be used to determine if the catheter is in the cavo-atrial junction but temperature or conductivity/impedance can be used to determine if the catheter has gone down an azygous or unintended vein branch.

In some embodiments, a camera may be used to optically determine the presence, and possibly the density, or number, of red blood cells. If a greater number of cells pass by, then the flow is stronger. If they are flowing in the opposite direction, then the flow has reversed direction, thus the catheter is proceeding in the incorrect direction.

These sensing modalities can also be combined with one or more (ECG) sensors to detect catheter placement. An ECG electrode or electrodes can be placed precisely either at the target location of the catheter tip (for example, in the superior ⅓ of the vena cava), or over the heart itself to detect an unnecessary over extension of the catheter. Alternatively, one or more ECG sensors may be incorporated into the device itself, for example, into a guidewire/stylet and/or catheter. Alternatively, ECG signals can be gathered with the same sensors or electrodes that are used to measure conductivity, temperature or other parameters. The received signal may alternate between ECG and conductivity for example, with or without breaks in between.

In any of the embodiments disclosed herein, the sensors may be located at or near the distal tip of, or along the length of a guidewire or stylet that passes through a vascular catheter. In any of the embodiments disclosed herein, the sensors may be located at or near the distal tip of, or along the length of a catheter.

One objective of some of the embodiments disclosed herein is to locate the device within the vasculature without the use of x-ray and/or fluoroscopy, and/or ultrasound and/or magnetic fields, and/or other imaging modalities.

Some embodiments disclosed herein may be specifically designed to be used with a sitting patient, or a patient with a pacemaker, or patients with specific conditions, etc.

One variation of a location detection system may generally comprise an elongate body having a lumen, wherein the elongate body is sized for introduction and translation through a catheter lumen. One or more openings may be located at or in proximity to a distal end of the elongate body and a sensor may be positioned at or in proximity to the distal end of the elongate body, wherein the sensor is configured to measure at least one parameter of a mixture of a first fluid and a second fluid after the first fluid is emitted from the one or more openings and into the second fluid when the distal end of the elongate body is advanced beyond a distal opening of the catheter lumen. A controller in communication with the sensor may also be included, wherein the controller is configured to receive a signal indicative of the at least one parameter of the mixture and is further configured to obtain a position of the sensor within a body of a subject based upon the signal.

One variation of a method of determining a location within a body of a subject may generally comprise introducing a first fluid through a lumen defined within an elongate body and through one or more openings located at or in proximity to a distal end of the elongate body after the distal end of the elongate body has been advanced beyond a distal opening of a catheter lumen, measuring a signal via a sensor of at least one parameter of a mixture of the first fluid and a second fluid after the first fluid is emitted from the one or more openings and into the second fluid, and determining via a controller in communication with the sensor a position of the sensor within the body of the subject based upon the signal.

### BRIEF DESCRIPTION OF THE DRAWINGS

FIG. 1 shows an embodiment of the vascular catheter navigation device navigating the human anatomy.

FIG. 2 shows an embodiment of the vascular catheter navigation device placed in the human anatomy.

FIG. 3 shows an embodiment of the vascular catheter navigation device.

FIGS. 4A-F show the influence of fluid flow direction on flow behavior of an injected fluid bolus with respect to the catheter tip before, during and after injection.

FIGS. 5A-5E show a variety of embodiments of the vascular catheter navigation device.

FIGS. 6A-6E show a variety of embodiments of the vascular catheter navigation device.

FIG. 7 shows an embodiment of the vascular navigation device with 2 sensors and multiple openings between the two sensors.

FIG. 8 is a schematic illustration showing fluid flow in different areas of the vascular system.

FIGS. 9A-9E show various embodiments of the vascular catheter navigation device.

FIGS. 9F-9J show distances between the fluid ports and the sensors.

FIG. 10 shows an embodiment of the vascular catheter navigation device which can be used with any catheter.

FIGS. 11A-I show various views of various embodiments of a stylet/guidewire version of the vascular catheter navigation device.

FIGS. 12-17 show various embodiments of the vascular catheter navigation device.

FIGS. 18A-E and 19A-E show possible graphical user interfaces of the device.

FIGS. 20A-E show various embodiments of the vascular catheter navigation device.

FIGS. 21A-C show different configurations of vascular catheter lumens and variations of embodiments of the vascular catheter navigation device which work with them.

FIGS. 22A-F show various embodiments of a guidewire/stylet component of the vascular catheter navigation device.

FIGS. 23A-C show an embodiment of the vascular catheter navigation device.

FIG. 24 shows data from an embodiment of the vascular catheter navigation device which uses optical reflection.

FIG. 25 shows an embodiment of the vascular catheter navigation device which uses optical reflection.

FIGS. 26 and 27 show a triple lumen device and a double lumen device respectively, with 2 fiber optic cables.

FIG. 28 shows an embodiment which includes a controller and a medium introduction mechanism.

FIG. 29 shows an embodiment of the injection mechanism.

FIG. 30 shows a version of the injection mechanism, or fluid pump, which may be disposable.

FIG. 31 shows an exploded view of the injection mechanism shown in FIG. 30.

FIG. 32 shows the embodiment of the injection mechanism shown in FIG. 30 where the cover is removed.

FIG. 33 shows the cover spring adjuster receptacles in underside of the cover.

FIGS. 34A and 34B show another embodiment of the injection mechanism.

FIGS. 35A and 35B show an embodiment of an injection mechanism which includes an add-on navigational component/controller component.

FIG. 36 is a block diagram of a data processing system, which may be used with any embodiments of the invention.

FIG. 37 shows an embodiment of the vascular catheter navigation device which includes sensors, or electrodes, and a diffuse exit port, for measuring conductivity.

FIG. 38 shows an embodiment with one pair of electrode sensors, and a diffuse exit port.

FIG. 39 shows an embodiment with a diffuse exit port, where the medium infusion lumen runs the length of the guidewire/stylet.

FIG. 40 shows an embodiment of the vascular catheter navigation device which includes a mesh or braid as a component of the diffuse fluid exit point.

FIG. 41 shows an embodiment with a diffuse exit port and which includes a spacer.

FIG. 42 shows an embodiment with a diffuse exit port and which includes a spacer.

FIGS. 43 and 44 show some possible embodiments of electrode pairs.

FIG. 45A shows the distal end of a vascular catheter navigation device with a diffuse exit port area.

FIG. 45B shows a porous sheet used to manufacture an embodiment of the catheter with a diffuse exit port area.

FIG. 45C shows the distal end of a vascular catheter navigation device with a diffuse exit port area.

FIGS. 46A-E show some examples of diffuse exit port designs.

FIGS. 47A-C show some more examples of diffuse exit port designs.

FIGS. 48A-D show how the injectate exit velocity impacts the sensor's ability to sense the injectate parameter within the vessel blood flow.

FIGS. 49A-49C show some embodiments of a trimmable vascular catheter with electrodes incorporated into the catheter.

FIG. 50 shows an embodiment of the vascular navigation device which uses electrodes as sensors.

FIG. 51 shows an embodiment of the vascular navigation device where the stiffener exits beyond the end plug.

FIG. 52 shows an embodiment of the vascular navigation device where the stiffener ends in a curved portion.

FIG. 53 shows an embodiment of the vascular navigation device with a reduced diameter exit port area.

FIG. 54A shows an embodiment of the vascular navigation device with a sleeve style exit port area.

FIG. 54B shows an embodiment of the vascular navigation device with a sleeve style exit port area.

FIG. 55 shows an embodiment of the vascular navigation device with a double later exit port area.

FIG. 56 shows an embodiment of the vascular navigation device where the core, or stiffener, includes the leads for the sensors/electrodes.

FIG. 57 shows an embodiment of the vascular navigation device where the stiffener is exposed at the distal end forming the distal most electrode.

FIG. 58A shows an embodiment of the vascular navigation device in which the electrode leads are separate coils embedded in the outer tubing of the stylet.

FIGS. 58B-58E show embodiments of the vascular navigation device with various electrode and opening configurations.

FIG. 59 shows another figure representing an embodiment of the vascular navigation device which is similar to that shown in FIG. 58A.

FIG. 60 is a close-up of the embodiment shown in FIG. 59.

FIG. 61 is a close-up of the embodiment shown if FIGS. 58 and 59.

FIG. 62 shows the close-up of FIG. 61 with the electrodes removed.

FIG. 63 shows an angled view of the electrode leads and their respective electrode bands.

FIG. 64 shows detail of an electrode band.

FIG. 65 shows an embodiment of an electrode band which includes a piercing mechanism.

FIGS. 66, 67 and 68 show embodiments of the of the navigation device which include an insulated sheath to limit the electrode area of the distal tip portion of the stylet/catheter.

FIG. 69A shows some example dimensions associated with some embodiments of the navigation device.

FIG. 69B illustrates the relevant distances between the distal tip of the catheter and the stylet/guidewire extending beyond the distal tip of the catheter.

FIG. 69C shows an embodiment with a small diameter wire, flexible coil, ball tip and hypodermic tube.

FIG. 69D shows a view showing a flexible tubing element.

FIG. 69E shows another view of the embodiment shown in FIGS. 69C and 69D.

FIG. 70A shows an embodiment of the navigation device where two electrodes are comprised of two wires.

FIG. 70B shows the stylet of FIG. 70A extended beyond the distal end of the catheter.

FIG. 70C shows some example dimensions of one embodiment of the navigation system.

FIG. 70D shows an embodiment where the electrodes are not stripped at the end but rather cut flush with the insulated coating.

FIG. 71A illustrates an embodiment of the navigation device with three pairs of wire pairs each placed approximately 120 degrees radially from each other around the stylet.

FIG. 71B illustrates an embodiment in which the electrodes, and stylet, are extended beyond the distal end of the catheter.

FIG. 72A shows an embodiment with the electrodes or electrode leads embedded into the wall of a catheter itself.

FIG. 72B shows an embodiment of the navigation device with multiple electrodes/leads embedded into the elongate body (catheter or stylet) wall.

FIG. 72C shows an embodiment where the electrodes/leads are formed via one or more spiral coils in the wall of the catheter or stylet.

FIG. 72D shows electrodes/leads embedded in the central divider of a dual lumen catheter.

FIG. 72E shows two concentric circular electrodes that are insulated from one another.

FIGS. 73A-73D show various embodiments of the navigation system which are incorporated into a catheter.

FIG. 74 shows the magnitude of the sensor signal within the anatomy.

FIGS. 75 and 76 show the relative magnitude of the signal from the distal and proximal sensors, as well as signal pulsatility.

FIG. 77 shows that signal magnitude, relative signal magnitude, and/or signal pulsatility can be used by the controller of the vascular navigation system to determine the location of the distal end of the device.

FIG. 78 shows an example of specific signatures received by the controller using some embodiments of the navigation device.

FIG. 79 shows a close up of a graph showing ionic dilution signatures and the transition of the signatures as the device tip enters different areas of the anatomy of a pig.

FIGS. 80A-80E show an embodiment of the vascular navigation device which is incorporated into a balloon catheter or stylet, such as an angioplasty catheter or stylet.

FIGS. 81A-C shows examples of vascular navigation systems in use with a pressure wire.

### DETAILED DESCRIPTION OF THE INVENTION

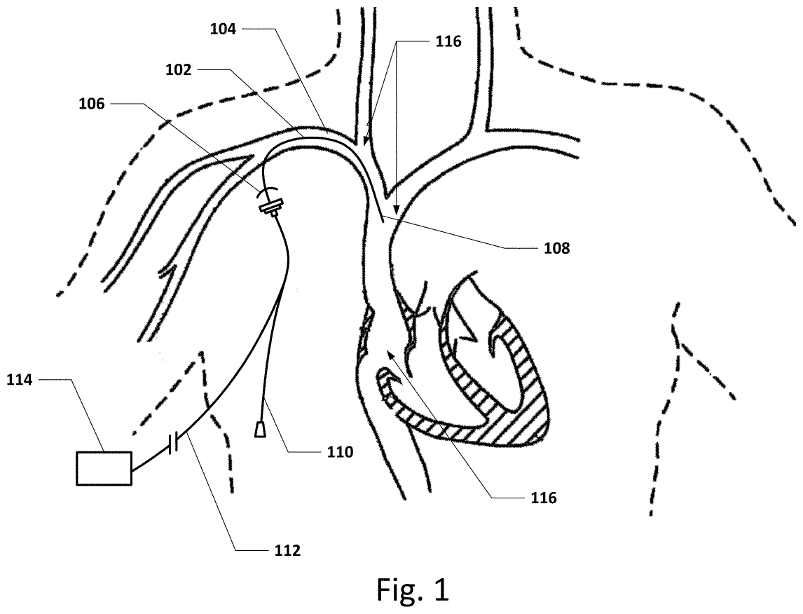

FIG. 1 shows an embodiment of the vascular catheter navigation device or system navigating the human anatomy. Vascular catheter navigation device 102 is shown in vein 104 of a patient. The vascular catheter navigation device has been inserted into the patient via insertion point 106. The insertion point is shown here in the patient's chest, however the insertion point may alternatively be the patients leg, arm or neck or other location. To navigate a standard vascular catheter into its desired location, several undesirable obstacles need to be avoided and/or overcome. For example, a vascular catheter may be mistakenly placed into an artery instead of a vein, a vascular catheter may venture down or up an incorrect branch of the vascular system, a vascular catheter may become lodged against a wall of a blood vessel, a vascular catheter may be advanced too far, either too close to the heart, into the heart or past the heart, or a vascular catheter may not be advanced far enough to reach its desired location, or may migrate to a less desirable location. A few of these hazard areas are labeled 116. Distal tip of vascular catheter navigation device is shown as 108. At the proximal end of vascular catheter navigation device, infusion or sampling lumen 110 is shown which is in fluid communication with opening or openings at or near the distal end of vascular catheter navigation device. Also shown is sensing port 112 which is in communication with controller 114. Sensing port 112 is in communication with one or more sensors (not shown here) at or near distal tip 108 of vascular catheter navigation device 102. Although one infusion/sampling lumen and one sensing port are shown here, multiple infusion/sampling and/or sensing ports may exist. Infusion lumen 110 may also be in communication with controller 114.

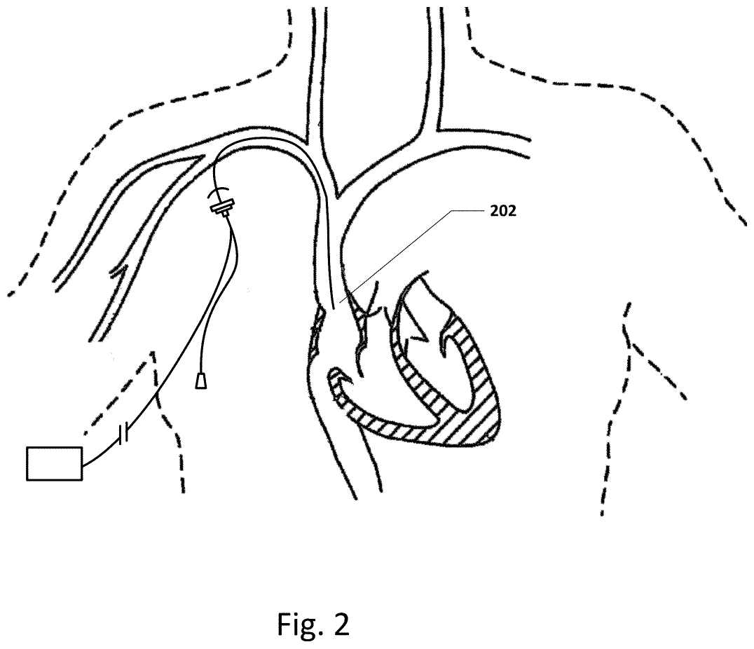

FIG. 2 shows an embodiment of the vascular catheter navigation device where the distal tip is placed in the superior vena cava/cavo-atrial junction (SVC-CAJ) 202.

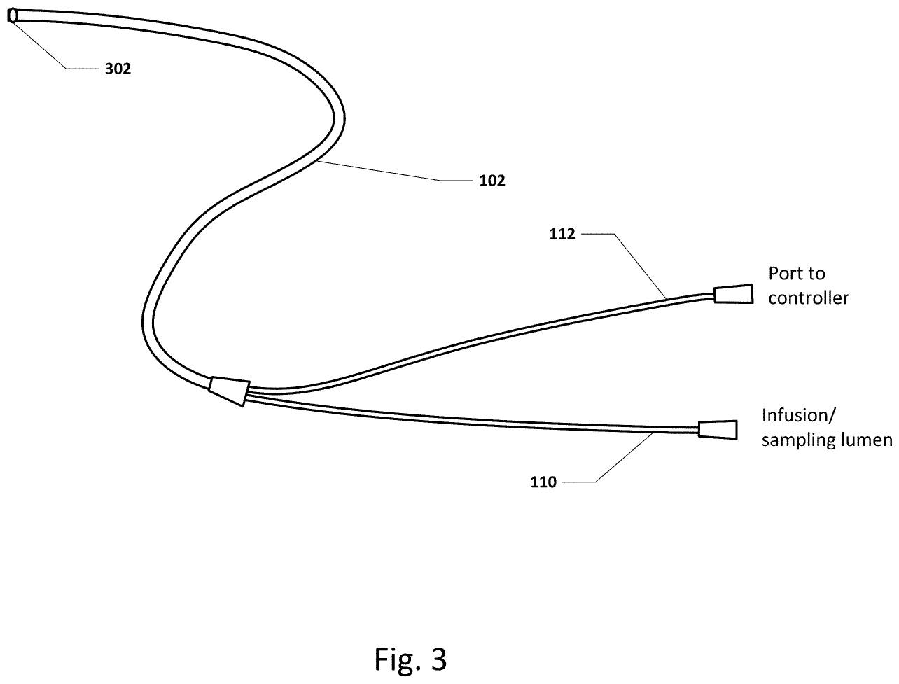

FIG. 3 shows an embodiment of the vascular catheter navigation device. The distal end of the vascular catheter navigation device is inserted into the appropriate access vein, and advanced along the vein to its target location. After the vascular catheter navigation device is inserted into the blood vessel, generally through a needle, catheter or sheath, sensing element 302 senses a parameter within the blood flowing through a blood vessel. A medium, such as fluid, with a measurable parameter, such as temperature, or conductivity, is injected through the device, and into the blood vessel. The sensor signals are communicated back to the controller where the sensor signal(s) are analyzed based on the sensor data over time, including data curve slope, magnitude, value, length, variability, pulsatility, phase, standard deviation, shape, pulsatility/fourier analysis etc. For example, the controller can determine whether the distal end of the vascular catheter navigation device is in an artery instead of a vein, based on magnitude and direction of blood flow around the vascular catheter navigation device by measuring and analyzing the measurable parameter. If the controller determines that the distal end of the vascular catheter navigation device is in an undesired position, an alert or other indicator may communicate with the user. For example, if the controller determines that the catheter is in an artery instead of a vein, a specific identifying signal may sound, including an audible, visual signal etc., instructing the user to retract the vascular catheter navigation device, and any other device, such as sheaths, catheters etc., and apply pressure to the blood vessel.

Similarly, the vascular catheter navigation device can sense when the distal end is in the incorrect branch of a vein, based on flow direction, and possibly flow profile and magnitude. When advancing the vascular catheter navigation device in the correct direction and in the correct vessel (toward the SVC-CAJ, in a vein), the blood flows over the vascular catheter navigation device from the more proximal end toward the distal end.

FIG. 3 shows one sensor 302, one sensor port 112 and one infusion/sampling lumen 110. However, more than one infusion/sampling lumen and/or more than one sensors may be present. In addition the port to the controller and the sampling lumen could be the same lumen and be incorporated into a single lumen device. The infusion and/or sampling lumen may also be connected to the controller.

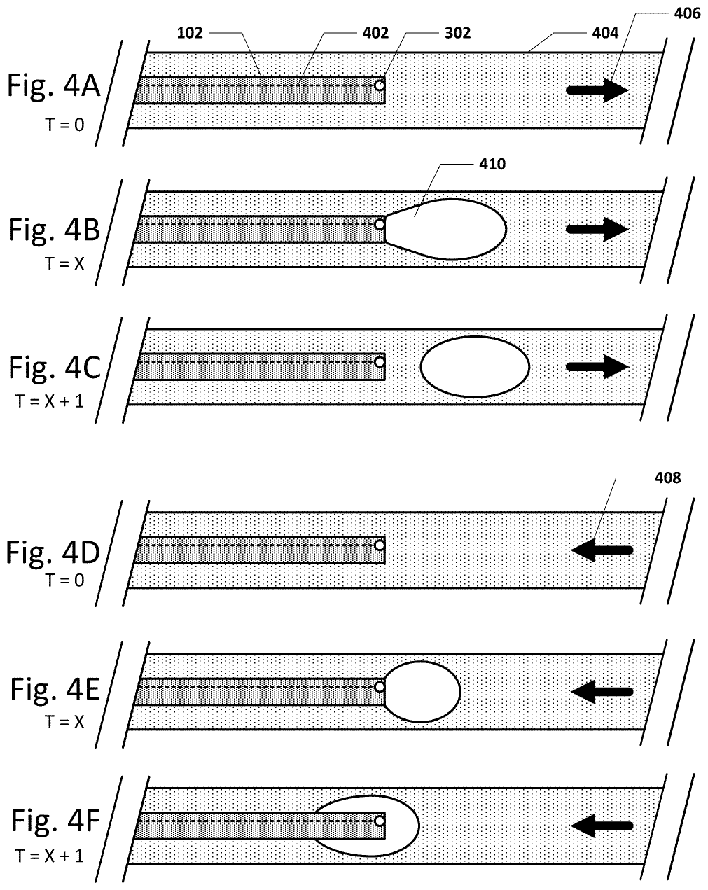

FIGS. 4A-F shows the influence of fluid flow direction on flow behavior with respect to the catheter tip before, during and after an injected fluid bolus. At time=0, device 102 is in vessel 404. Device 102 includes sensor 302. Sensor 302 is designed to measure a parameter of blood and/or the injection medium. The controller (not shown) is in communication with sensor 302 via lead connector 402 which, in this example, runs the length of the catheter back to the controller. Sensor 302 and connector or lead 402 may be incorporated into the vascular catheter or may be incorporated into a stylet that runs through the catheter. Medium 410 is introduced into the vessel at time=x. For example, the medium may be saline at a temperature which is different than that of the body. The parameter measured by the sensor in this example would be temperature, but could be any parameter, such as conductivity. After the injection, at T=x+1, blood flow will mix the medium with the blood flow. FIGS. 4A-C show the device in in-line blood flow. Where blood flow 406 flows away from the catheter, the bolus of medium 410 travels away from the catheter tip and away from the sensor. FIGS. 4D-F show the device in counter blood flow, such as in an artery. Where blood flow 408 flows toward the catheter, the bolus of medium 410 travels toward and over the catheter tip. This example shows a bolus of fluid, but a stream of fluid may also be used.

Depending on the location of the sensor(s), different temperature, or parameter, profiles may be measured over time/location. Variables in flow rate, direction, turbulence, etc. will affect the mixing of blood and medium and affect the profile of the parameter, in this example, temperature, over time. In this way, the system can determine blood flow direction and characteristics at or near the catheter tip.

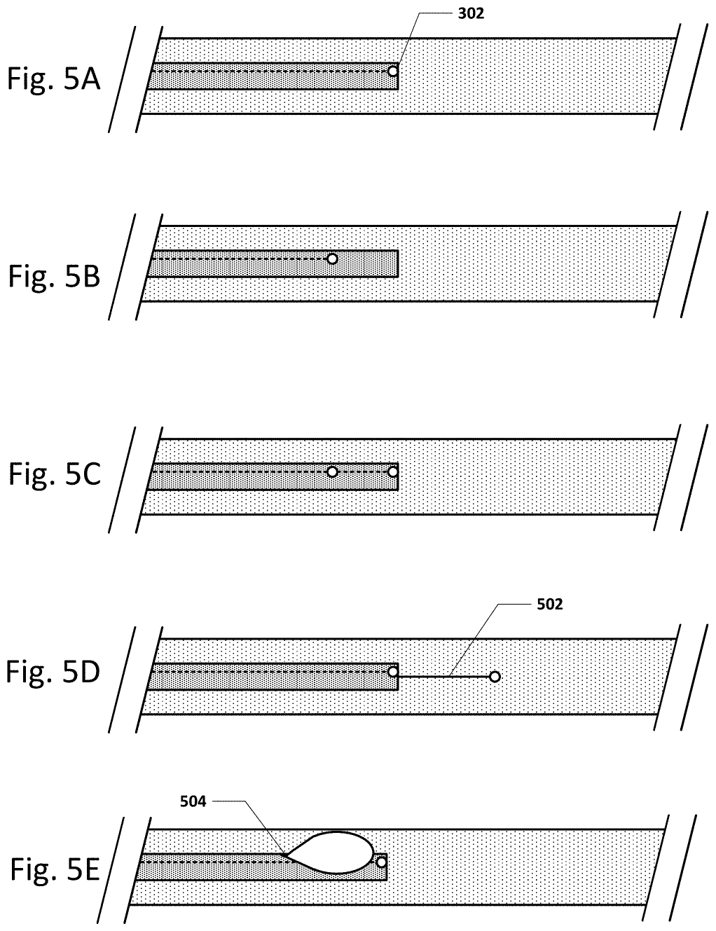

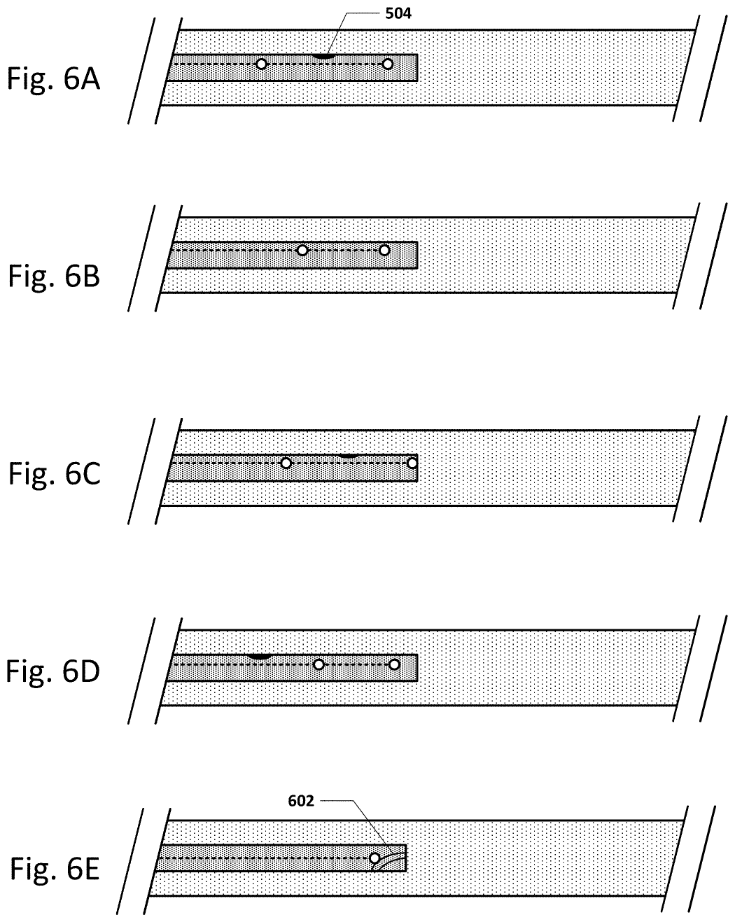

FIGS. 5A-5E and 6A-6E show several example embodiments of the vascular catheter navigation device. FIG. 5A shows an embodiment with sensor 302 at the catheter tip. FIG. 5B shows an embodiment with the sensor near, but not at, the catheter tip. This configuration may prevent the sensor from measuring the parameter during introduction of the medium from the catheter tip, allowing better distinction between flow directions. FIG. 5C shows an embodiment with 2 sensors, one at the catheter tip, and one near, but not at, the catheter tip. Sensor readings at different positions will vary based on fluid flow direction, characteristics, profile etc. A sensor near, but not at, the catheter tip may be from about 0.05 cm to about 2.0 cm back from the tip. Alternatively, a sensor near, but not at, the catheter tip may be from about 0.75 cm to about 1.25 cm back from the tip. FIG. 5D shows an embodiment where a sensor is on guidewire or stylet 502. Stylet 502 may move freely within the catheter allowing one or more sensors to be placed at a distance from the catheter tip. In addition, the guidewire/stylet may be removed after catheter placement. In this embodiment, the catheter may also include a sensor, as shown here. FIG. 5E shows an embodiment with opening 504 which is near, but not at, the catheter tip. This opening may be in fluid communication with a separate medium introduction lumen or infusion lumen, or the same lumen as that of the distal opening. This specific medium introduction lumen may exit at the catheter tip. An opening near, but not at, the catheter tip may be from about 0.25 cm to about 2.0 cm back from the tip. Alternatively, an opening near, but not at, the catheter tip may be from about 0.75 cm to about 1.25 cm back from the tip. The medium introduction lumen may be in the catheter or may be within the stylet. More than one injectate medium may be introduced either through the same lumen or through separate lumens of the device.

FIG. 6A shows an embodiment with an opening between two sensors, both of which are near, but not at, the catheter tip. FIG. 6B shows an embodiment with more than one sensor near, but not at, the tip of the catheter. FIG. 6C shows an embodiment with an opening between two sensors, one of which is at the catheter tip. FIG. 6D shows an embodiment which includes an opening proximal to 2 sensors. FIG. 6E shows an embodiment with channel 602. Channel 602 allows fluid to flow within the catheter, in proximity to a sensor within the catheter.

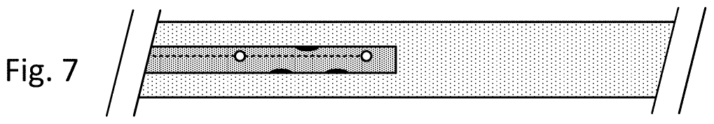

FIG. 7 shows an embodiment of the vascular navigation device with 2 sensors and multiple openings between the two sensors.

It is apparent that numerous variations of these and other embodiments of the vascular catheter navigation device are envisioned. For example, sensors, openings, channels etc. may be on different sides of the catheter and/or guidewire/stylet. Sensors, openings and channels are shown here at or near the catheter tip, however, they may be located anywhere along the catheter and/or guidewire/stylet.

Different sensor configurations will result in different parameter curve signatures in different vascular locations. For example, a single sensor will give a different set of curves than will a system with 2 sensors. The distance of the sensor(s) from the infusion exit site will also provide different curves. Different infusion rates, infusion volumes, infusion types (bolus vs. stream), infusion pressures, infusion velocities etc., will also provide different curves and thus different anatomical signatures. Different aspects of the curves may be analyzed by the controller to determine vascular location. These may include, but are not limited to, slope, magnitude, value, length, variability, pulsatility, phase, standard deviation, shape, area under the curve, Fourier transform, frequencies, harmonics, etc. In some embodiments, certain frequencies in the data may be filtered out, including those relating to the heartbeat, system noise, tissue conductance, etc.

In some embodiments there is one sensor and therefore one parameter vs. time/location curve. In some embodiments there are two or more sensors and therefore two or more parameter vs. time/location curves. In some embodiments, the infusion exit port is near the more proximal sensor or sensors. In some embodiments the infusion exit port is proximal or distal to the sensor or sensors. In some embodiments the infusion exit port is between the sensors. In some embodiments, one or more than two sensors may be used.

Note that parameter curves may appear different, in different anatomy, and based on the design of the vascular catheter navigation device. For example, the curve may be different for different sensor locations with respect to the fluid exit port. The curve may depend on the type of sensor or the fluid injection rate. The curve may depend on the initial parameter level of the injection fluid. Other design factors may also result in different parameter vs. time/location curve shapes.

In addition, calibration of the sensor vs. time/location curves may be performed by the controller. For example, a baseline measurement may be derived after insertion of the system, or at other points during use of the system. For example, a baseline measurement may be taken in the blood vessel before any injection fluid is injected, or at a particular injection rate. A baseline measurement (a measurement taken without any fluid injection into the system) may be used in the controller's analysis of the data to determine the location of the vascular catheter navigation device within the anatomy.

Various properties of the parameter vs. time curves may be analyzed to determine the location of the vascular catheter navigation device. For example, curve amplitude, noise, standard deviation, shape, slope, value, area under the curve, Fourier transform, frequencies, harmonics, etc. of one or more curves may be used to determine the vascular catheter navigation device location within the vasculature. These same parameters may be compared between and among multiple parameter vs. time/location curves to determine vascular catheter navigation device placement location. For example, the location, relative location, magnitude, and/or relative magnitude of peaks (positive or negative) of the curves may be used to determine vascular catheter navigation device location. In addition, the difference between amplitude, noise, standard deviation, shape, slope, value, area under the curve, and/or Fourier transform, harmonics, frequencies of the data from the multiple sensors may be used to determine vascular location. Depending on droplet size and/or infusion rate, an area under the curve, or Fourier transform may be used to analyze the parameter vs. time curve and thus vascular location. Additionally, a maximum, or a number of maxima, may be relevant.

The term “droplet” used herein may mean a drop, a bolus, a stream, an intermittent stream, etc. when referring to the injectate.

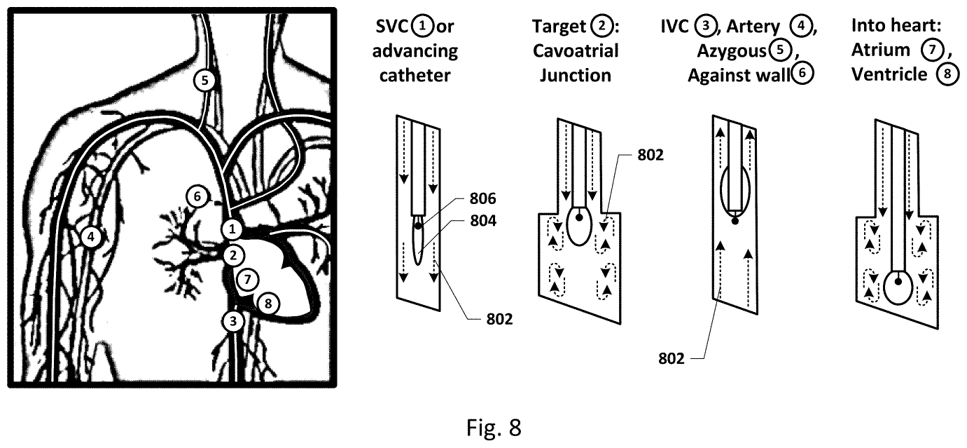

FIG. 8 is a schematic showing fluid flow in different areas of the vascular system representing desired (correct) and undesired (incorrect) device placement. Arrows 802 show blood flow direction. Areas 804 show fluid (such as saline) infusion. Note how the different anatomical locations will yield different flow conditions and thus different dissipation patterns of the fluid infusion. Although 1 sensor 806 is shown here, two, or three, or four or five or six or more may be used, in this, and any other embodiments disclosed herein.

Note that several embodiments disclosed herein may mention a particular type of sensor and measured parameter, such as a sensor measuring temperature. However, any of the embodiments disclosed herein may use any type of sensor (or more than one type of sensor) which measures that sensor's parameter. For example, embodiments that disclose sensors measuring temperature, may alternatively, or additionally include conductivity sensors measuring conductivity. Embodiments which mention the controller using data from a particular type of sensor, may alternatively or additionally use data from another type of sensor.

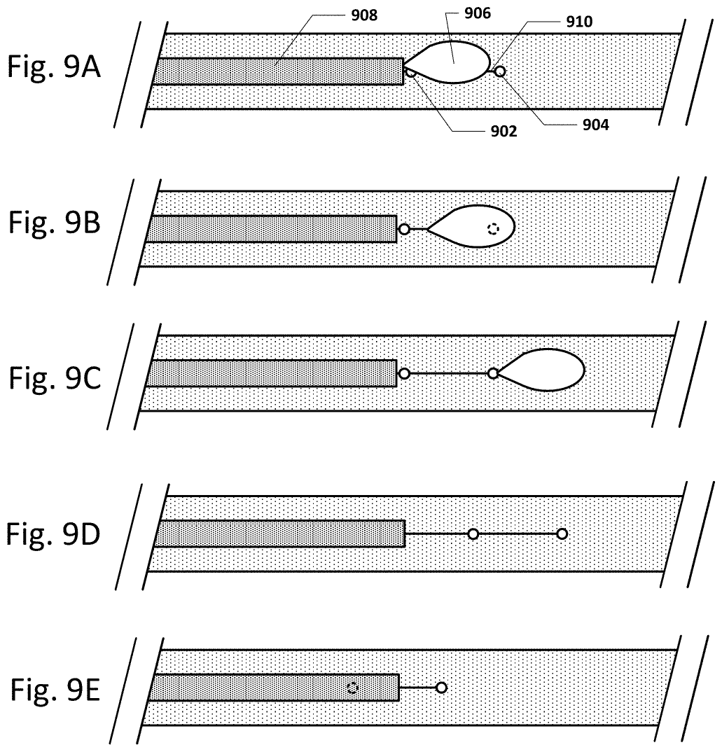

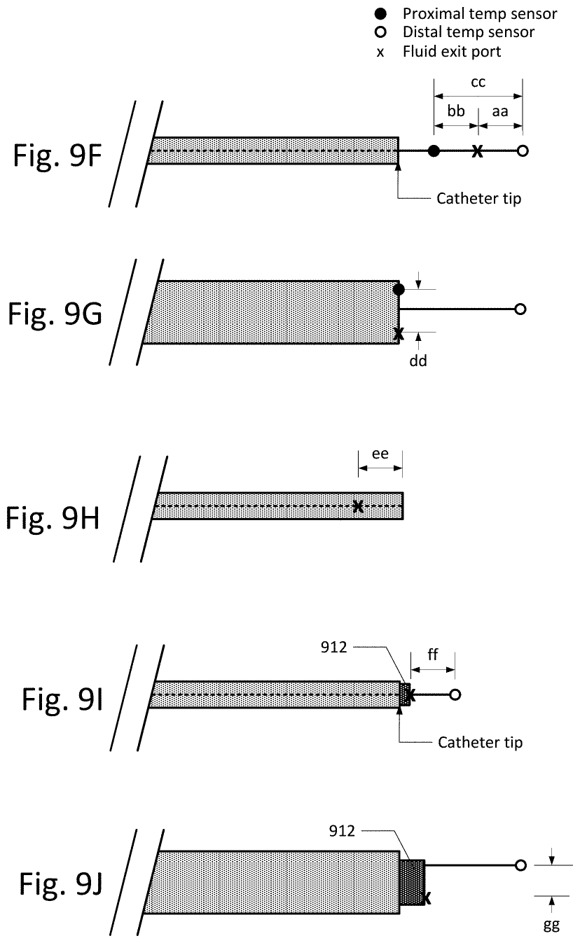

FIGS. 9A-9E show various embodiments of the vascular catheter navigation device where two sensors, or other types of sensors are on the guidewire/stylet. FIG. 9A shows stylet 910 with proximal sensor 902 and distal sensor 904. The injectate 906 in this embodiment exits at the distal tip of catheter 908, proximal, or near to proximal sensor 902. Alternatively, the injectate may be injected through a lumen of the guidewire/stylet. Although 2 sensors are shown here, one, or more than 2 may be used.

FIG. 9B shows an embodiment where the injectate is injected through the stylet/guidewire and exits between the two sensors. FIG. 9C shows an embodiment where the injectate is injected through the stylet/guidewire and exits near or distal to the distal sensor. If one sensor is used, the fluid injection exit port may be either proximal to, or distal to the sensor.

FIGS. 9D and 9E show an embodiment with two sensors on the stylet/guidewire where the guidewire is able to be moved with respect to the end of the catheter. This embodiment may be used to alter the sensing and/or injectate exit location with respect to the tip of the catheter.

For example, in some embodiments, the stylet/guidewire may include both the injection lumen (i.e. the stylet/guidewire may be hollow) and a sensor so that it may be positioned in the anatomy first and/or independently of the vascular catheter. For example when jugular access is being used for catheterization. Once the stylet/guidewire is placed, the vascular catheter may be advanced so that the distal tip of the catheter is at a known position relative to the distal tip of the stylet/guidewire. The stylet/guidewire may then be removed.

FIGS. 9F through 9H show distances between the fluid exit port and the sensor(s) and distances between the catheter/stylet tip and the sensor(s)/ports. FIG. 9F shows the axial distance aa between the injectate exit or port, and the distal or singular sensor. The axial distance bb is the distance between the fluid exit port and the proximal sensor. The axial distance cc is the distance between the distal sensor and the proximal sensor. These distances may be positive or negative. Although 2 sensors are shown here, the device may have one sensor or more than 2 sensors.

Distance aa may be about 0 mm. Alternatively, distance aa may be a range of about 0 mm to about 0.5 mm, or about 0 mm to about 1 mm. Alternatively, distance aa may be a range of about 0 mm to about 2 mm. Alternatively, distance aa may be a range of about 0 mm to about 3 mm. Alternatively, distance aa may about 3 mm to about 5 mm. Alternatively, distance aa may about 5 mm to about 10 mm. Alternatively, distance aa may be a range of about 0 mm to about 100 mm. These distances may alternatively be negative. For example, distance aa may be about 1 mm or may be about −1 mm. In the case of 1 mm, the distal sensor will be distal to the fluid exit port. In the case of −1 mm, the fluid exit port will be distal to the distal sensor. This is true for all dimensions provided in association with FIG. 9F-9H.

Distance bb may be about 10 mm. Alternatively, distance bb may be a range of about 0 mm to about 10 mm. Alternatively, distance bb may be a range of about 8 mm to about 12 mm. Alternatively, distance bb may be a range of about 5 mm to about 15 mm. Alternatively, distance bb may be a range of about 1 mm to about 100 mm. Alternatively, distance bb may about 3 mm to about 5 mm. Alternatively, distance bb may about 5 mm to about 10 mm. Alternatively, distance bb may be a range of about 0 mm to about 100 mm. These ranges may also be negative distances.

Distance cc may be about 10 mm. Alternatively, distance cc may be a range of about 0.0 mm to about 5 mm. Alternatively, distance cc may be a range of about 5 mm to about 15 mm. Alternatively, distance cc may be a range of about 15 mm to about 20 mm. Alternatively, distance cc may be a range of about 1 mm to about 100 mm.

Distance dd in FIG. 9G is the distance between the fluid exit port and either the distal or proximal sensor. The distance is shown with respect to the proximal sensor here, but distance dd may apply to either. Alternatively, only one sensor may be present. Distance dd may be about 0.75 mm. Alternatively, distance dd may range from about 0.25 mm and 1.5 mm. Alternatively, distance dd may range from about 0.1 mm and 5 mm.

FIG. 9H shows the axial distance ee between the fluid exit port and the end of the catheter and/or stylet. Distance ee may be about 0 mm. Alternatively, distance ee may range from about 0 mm and about 1 mm. Alternatively, distance ee may range from about 0 mm and about 3 mm. Alternatively, distance ee may range from about 0 mm and about 5 mm. Alternatively, distance ee may range from about 5 mm and about 10 mm. Alternatively, distance ee may range from about 0 mm and about 100 mm. These distances may be positive or negative.

FIG. 9I shows an embodiment of the vascular catheter navigation device which includes only one sensor and includes a conduit in the system. The conduit may serve as a “plug” on the guidewire/stylet that blocks a lumen of the catheter to direct the flow of injected fluid through the catheter from the flow passages. Conduit 912 incorporates the injectate exit port shown by an X. Distance ff shown here is the longitudinal distance between the fluid injectate exit port of the conduit and the sensor.

FIG. 9J shows an embodiment similar to that in FIG. 9I where the distance gg represents the radial distance between the fluid injectate exit port of the conduit and the sensor.

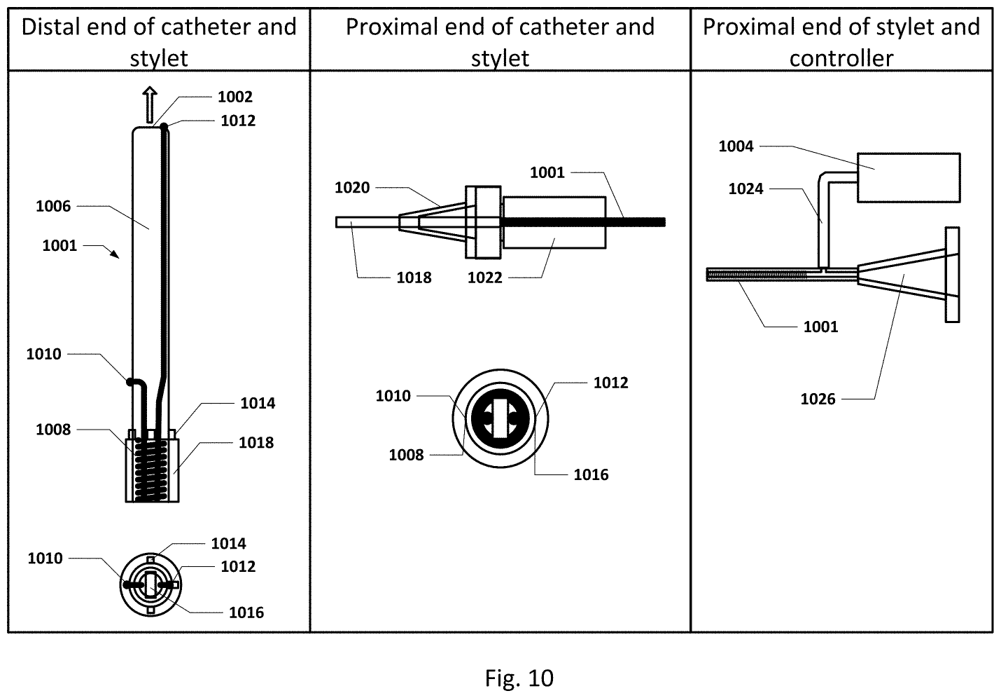

FIG. 10 shows an embodiment of the vascular catheter navigation device which can be used with any catheter, or in other words, where the sensor(s), the injectate lumen, the controller, and locking mechanism are included with the stylet/guidewire. FIG. 10 shows an embodiment with two sensors, distal sensor 1012 and proximal sensor 1010, and injectate exit port 1002 as part of guidewire/stylet 1001. Alternatively, the stylet/guidewire may only have one sensor, or may have more than two sensors. The stylet/guidewire may include features 1014 to help align the stylet/guidewire and the catheter. This embodiment may include a tip portion 1006, such as a molded urethane, nylon, silicone, or other polymer portion, for embedding the sensor(s). Also shown here is an optional guidewire/stylet coil 1008 and the distal tip of catheter 1018. In the cross sectional view, injection lumen 1016 can also be seen.

This embodiment may include torque or locking device 1022 which may be used to lock the stylet to the proximal end of the catheter, for example using luer lock 1020 at the proximal end of catheter 1018. The torque/locking device may be locked to the stylet/guidewire so that the stylet/guidewire won't move with respect to the vascular catheter. Controller (not shown) may include and/or control an infusion mechanism via fluid port 1026 as well as read data from the sensor(s) via sensor port 1004. The controller may be located near the proximal end of the stylet, or may be located several inches or feet from the proximal end of the stylet. sensor leads 1024 are also shown. The infusion may be steady or intermittent or consist of boluses.

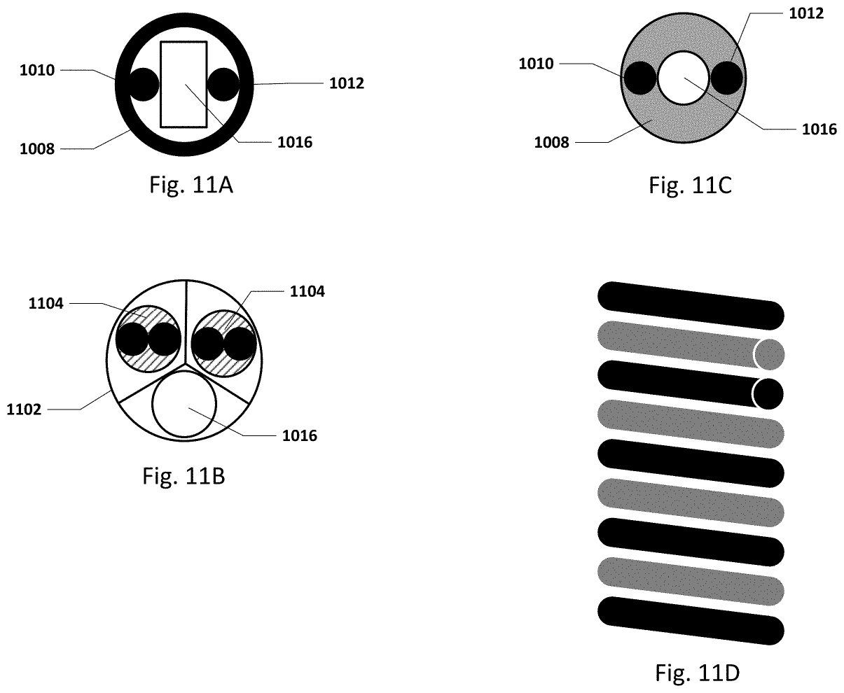

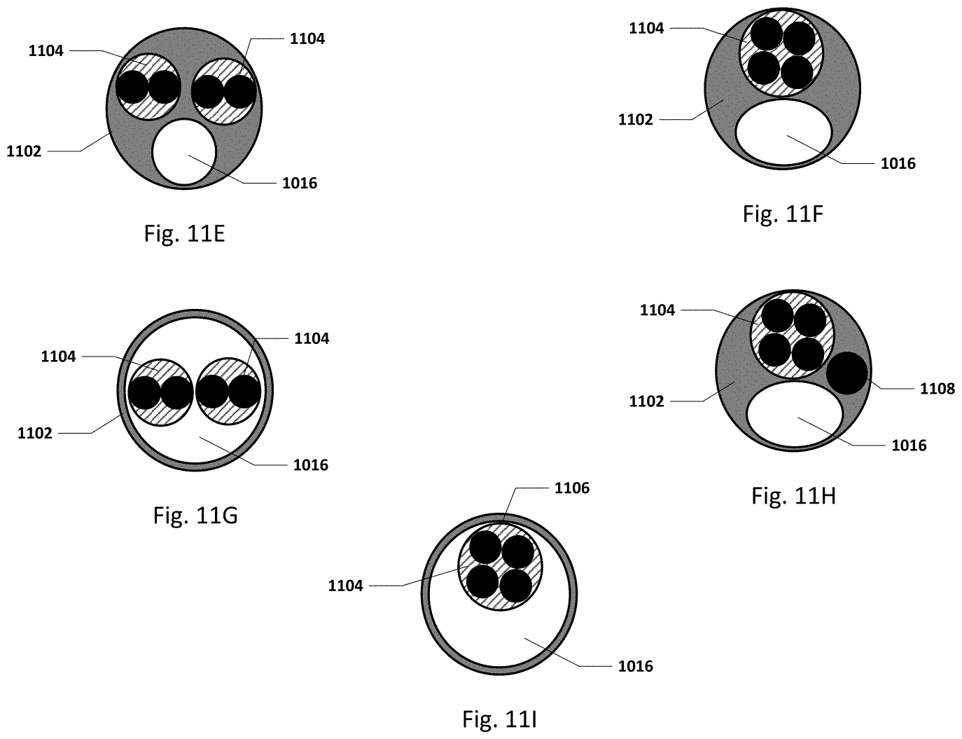

FIGS. 11A-I show various views of various embodiments of a stylet/guidewire version of the vascular catheter navigation device.

The stylets shown in 11A-11I and some other embodiments serve several functions, including: 1) Stiffening of the catheter to aid in insertion 2) providing a medium for fluid delivery and 3) providing a channel for the leads for the sensor or sensors. FIG. 11A is a cross section of the stylet such as that shown in the embodiment of FIG. 10. Two sensors are shown here, but the device may include one, or more than two sensors.

FIG. 11B shows an embodiment of a stylet which includes three components in a triple lumen, heat shrink, and/or tubing housing 1102 which contains two sensors 1104 and fluid lumen 1016. Alternatively, one or more than two sensors may be present.

FIG. 11C shows an embodiment in which the stylet coil is made all, or in part, out of the sensor wires or leads. FIG. 11D is a side view of the embodiment shown in FIG. 11C.

FIG. 11E shows an embodiment including an extrusion, or tube, (metal or plastic) which houses two sensors as well as a fluid lumen. Alternatively, one or more than two sensors may be present.

FIG. 11F shows an embodiment including an extrusion, or tube, (metal or plastic) which houses multiple sensor leads within one bundle as well as a fluid lumen.

FIG. 11G shows an embodiment including a thin walled extrusion, or tube, where the sensor leads are surrounded by the fluid lumen. One, two, or more than two sensors may be present.

FIG. 11H shows an embodiment including an extrusion, or tube, (plastic or metal) which includes multiple sensors, a fluid lumen, as well as stiffener 1108 which may be a wire or a rod. One, two, or more than two sensors may be present.

FIG. 11I shows an embodiment including an extrusion, or tube, (plastic or metal) which includes a sensor lead bundle as well as a fluid lumen. The sensor lead bundle exterior may be made of similar material to the outer extrusion which enables optional chemical or heat formed bond or weld 1106. One, two, or more than two sensors may be present.

In some embodiments, it may be important to either fix, or precisely control, the distance between the catheter tip and the guidewire/stylet, or be able to determine the distance between the catheter tip and the guidewire/stylet. It may also be important to able to fix the location of the injection with respect to a sensor or to know the distance between the location of the injection exit port and a sensor. The distance between the exit port and the sensors will have an effect on the parameter profile during fluid infusion. These distances may be fixed across patients and scenarios, or may be different for different patient types and different scenarios. For example, the distance may be different depending on the vasculature being accessed. The distance may be different for patients of different weight, size, body mass index, health, age, sex, heart condition, or other patient characteristics. The distance may be different for different catheter sizes, catheters with different numbers and shapes of catheter lumens etc.

In some embodiments, the stylet/guidewire is fixed, or locked, with respect to the catheter tip using a torque device near the proximal end of the catheter as shown in FIG. 10.

In some embodiments, the user determines the relative alignment of the catheter and stylet/guidewire by sight and then measures the relative distance from two values.

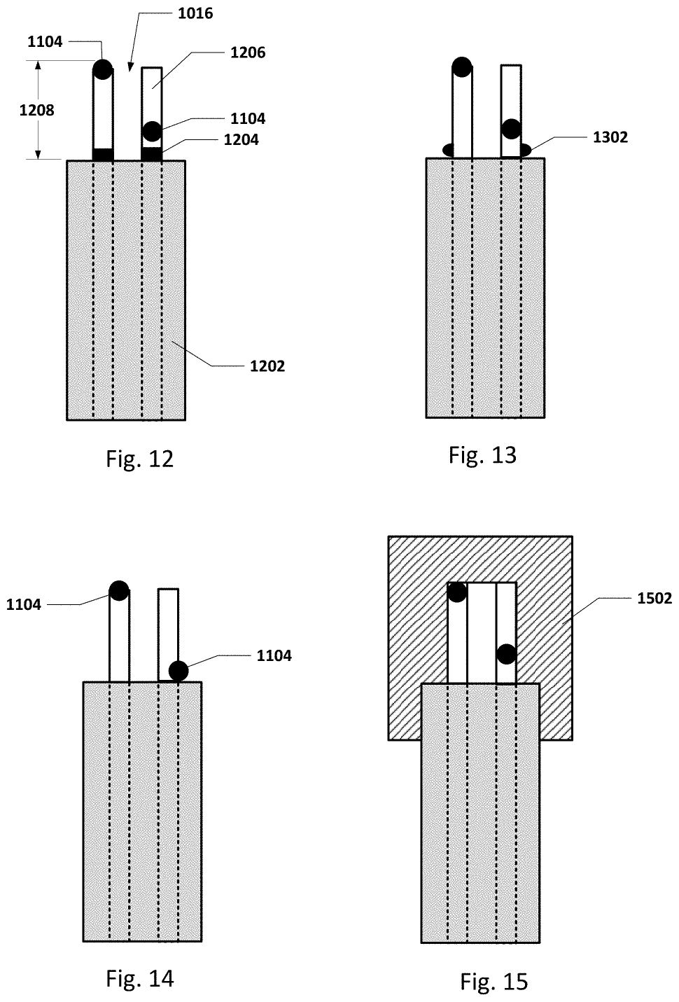

FIGS. 12-17 show various embodiments of the vascular catheter navigation device which include various registration techniques to either fix, or know, the distance between the sensor or sensors on the stylet, and the catheter tip or fluid injection point.

FIG. 12 shows an embodiment with an indicator on the stylet/guidewire which is a fixed and known distance from a sensor. In this embodiment, the user aligns the tip of catheter 1202 with indicator, or mark 1204, on stylet 1206 before insertion into the patient. The relative distance 1208 of the catheter tip to the tip of the stylet may be locked, preferably at the proximal end, using a torque device, a locking rotating hemostasis valve, a tuohy-borst valve, or other locking mechanism, before the catheter is inserted into the patient. The indicator on the guidewire/stylet may be a visible mark, such as a red stripe or dot, or a tactile mark, such as a bump or groove, or other type of indicator.

FIG. 13 shows an embodiment with raised area, or bump 1302, on the stylet which is a fixed and known distance from the distal sensor. This allows the user to align the tip of the catheter with the bump on the stylet, either visually, or by tactile feel. This alignment may be done outside the body or inside the body. In some embodiments, the bump is small or soft enough that the stylet may be removed from the catheter after placement in the anatomy.

FIG. 14 shows an embodiment similar to that shown in FIG. 13 where a sensor 1104 acts as the bump on the stylet.

FIG. 15 shows an embodiment where jig, or block, or aligner 1502, is used to align the tip of the catheter a fixed and known distance from the tip of the stylet. The relative location of the catheter with respect to the stylet is then locked, at the proximal end, using a torque device, a locking rotating hemostasis valve, a tuohy-borst valve, or other locking mechanism, and/or at the distal end, using a securing stylet, conduit, or both. Jig or block 1502 may itself be adjustable so that it can align the fluid exit port (here the distal end of the catheter) with the sensor for a variety of different lengths.

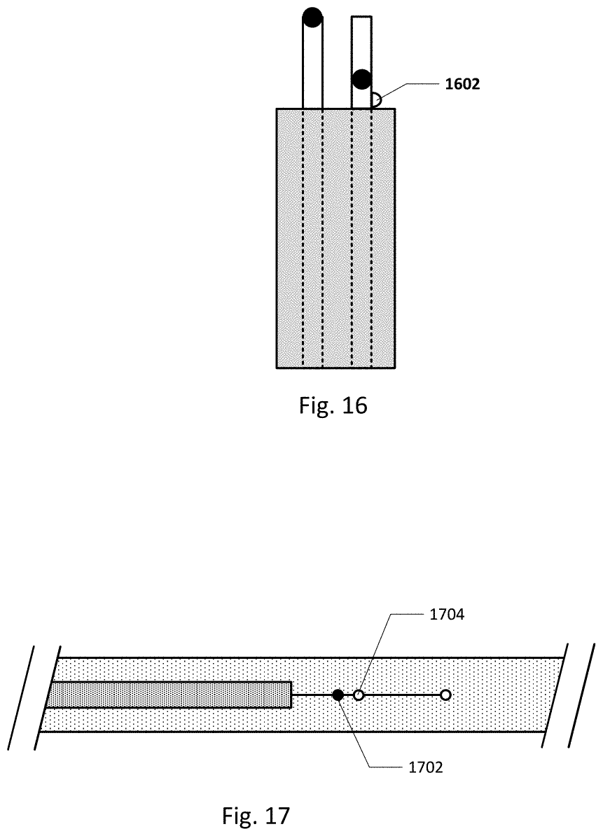

FIG. 16 shows an embodiment similar to that shown in FIG. 13 where inflatable balloon 1602 is used as the bump to align the catheter and the stylet. The balloon may be annular or on one or more sides of the stylet. The balloon may be inflated for use during alignment, and either left inflated during placement, to lock the stylet in position with respect to the catheter, or deflated during placement (where the catheter and stylet have been locked to each other using a torque or valve). In this embodiment, the stylet or catheter will include an inflation lumen to inflate and deflate the balloon. The balloon may be deflated for removal of the stylet after placement of the catheter.

FIG. 17 shows an embodiment of the vascular catheter navigation device which includes a sensor 1702 which can sense when it is inside the catheter tip during use. For example the sensor may be magnetic, ultrasound, light, temperature, etc. In some embodiments, proximal sensor 1704 is used as a sensor to determine when the proximal sensor is inside the catheter tip. The parameter vs. time/location curve shape after injection of injectate will show a specific profile when the sensor is just inside the catheter tip, and can be used to identify this alignment. This embodiment may include one, two, or more sensors.

In some embodiments, controlling the flow patterns of the injectate exit may be important. to achieve consistent results. It may also be important to contrast the flow of the injectate with that of the blood flow within the vasculature/heart. The flow of the injectate may be purposefully made either more laminar or more turbulent to achieve these goals. Some embodiments may include features that direct the flow and are a part of the catheter or stylet. These features may be surface features, like dimpling, or an orange peel finish, that change the surface finish of the catheter or stylet. These features may be part of the OD of stylet/temp sensors or ID of fluid lumen or both.

Note that several embodiments disclosed herein show 2 sensors. In any of these embodiments, one, two, or more sensors may be used.

In some embodiments the outer diameter (OD) of the stylet is around 1 mm or less. In some embodiments the OD of the stylet is around 0.5 mm or less. In some embodiments the OD of the stylet is around 1.5 mm or less. In some embodiments the stylet could range in OD from about 0.2 mm to about 5 mm.

In some embodiments, where the catheter is double or triple lumen, the stylet functionality may be broken into distinct parts (fluid, stiffener, sensing leads) etc. and multiple stylets may be used in multiple lumens of the catheter.

Many types of temperature sensors may be used in any of the embodiments disclosed herein, including thermocouples, fiber optic, resistive, bimetallic, thermometer, state-change, silicon diode, thermistors, optical temperature measurement (infrared or otherwise), mercury thermometers, manometers, etc.

In addition to infusing fluids, as disclosed elsewhere herein, other methods to create a thermal change at or near the tip of the catheter/stylet may be used. Fluids at a temperature higher than body temperature may be introduced, a resistive heating element, or a piezo electric cooling element, etc. may be included in the catheter, on the catheter, on the guidewire/stylet, or at the injector, outside of the body. Alternatively, the injected fluid may be at a different, although not strictly controlled, temperature than body temperature and this temperature difference (between body temperature and injectate temperature) is measured and tracked by the controller.

In embodiments with a resistive heating element, the resistive heating elements may be on the catheter or on a stylet. In embodiments where it is on the catheter it may be on the outside of the catheter or on the inside of one or more lumens of the catheter. Alternatively, it may be on the guidewire/stylet. In embodiments where it is on the guidewire/stylet, it may be within the catheter lumen, partially within the catheter lumen, or external to the catheter lumen, where it is exposed to blood. Embodiments that heat/cool blood may not require the injectate fluid.

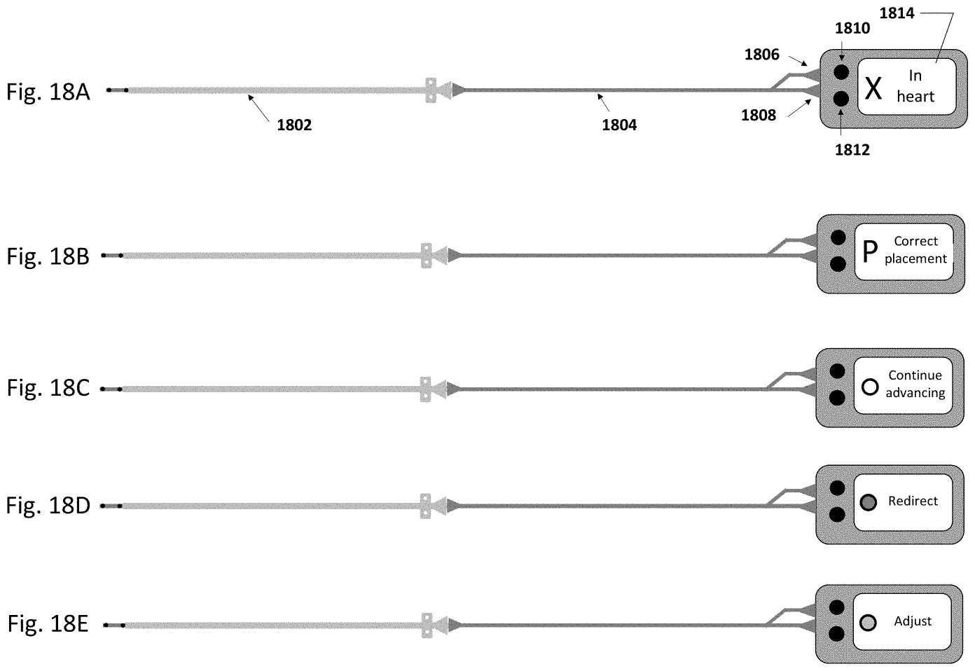

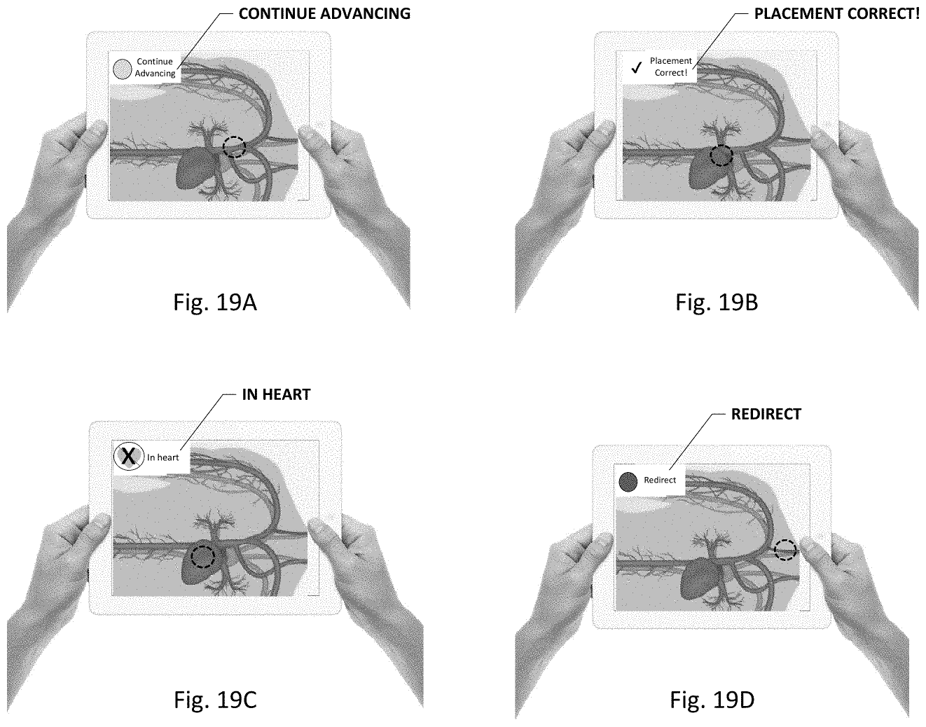

As shown in FIGS. 18A-E and FIGS. 19A-E. A graphical user interface may be displayed in the form of a small screen/display 1814, a large screen, a projection, in virtual reality or augmented reality goggles, etc. The major categorization of user interactions may have any combination of user alerts: 1) icon 2) color of icon or warning light 3) auditory tone that accompanies the alert 4) visual map of the body which matches the location of the catheter tip and with the type of alert 5) written phrase or word on the display indicating the status or alert, vibration, etc. The categories may be the following: 1) ‘Continue Advancing’, which means that that the catheter tip is advancing through a peripheral vein or has rounded the bend and is approaching the superior vena cava. This mode will be accompanied by visual and auditory feedback indicating a positive state such as green lighting and iconography and a positive tone. 2) ‘Placement Correct’, with the checkmark iconography shows that the tip has arrived at the proper location—the cavo atrial junction for PICC lines, or perhaps another location for another type of catheter insertion. Positive tone and lighting may also accompany this state. 3) If the catheter encounters an opposing flow, the warning, ‘Redirect’ may appear. This is the warning if the catheter advances down an azygous branch, advances into the IVC, or has been placed in an artery. Since this is not a positive state, lighting or iconography that is red, yellow or orange may accompany this state along with a tone which depicts that a non-favorable situation is in effect. A less pleasant frequency, pitch and tone may accompany. 4) if the catheter is in the heart, either in the atrium or the ventricle, the user may be alerted with the heart icon and/or “In Heart” warning. A negative color and tone may accompany this state. 5) if the catheter tip is up against a wall of a vein, or has an obstruction of some kind the “Adjust” warning may display. A negative color and tone may accompany this state. Also shown in FIGS. 18A-E and 19A-E is catheter 1802, stylet 1804, sensor adapter 1806, fluid adapter 1808, prime button 1810, and insertion/tracking button 1812.

The graphical user interface (GUI) may display in real time the location of the tip of the catheter relative to the 3D space through which it is navigating. The graphical user interface shown in FIGS. 18A-E and 19A-E are two dimensional, however some embodiments include 3D displays which may also communicate the information in three dimensions.

Note that although some embodiments disclosed herein incorporate the sensor(s) into the vascular catheter, the vascular catheter navigation device may be a stand-alone device which fits inside a vascular catheter, and can be removed once vascular catheter placement has been completed. The vascular catheter navigation device, for example, may serve as a stylet or guidewire for a standard vascular catheter.

The cross sectional area and shape of the flow passages will, at least partially, determine flow velocity exiting the device. The number of flow passages will also affect the flow parameters of the fluid exiting the device. Preferably, the fluid infusion rate may be about 2-3 ml/min. Alternatively, the fluid infusion rate may be about 3-5 ml/min. Alternatively, the fluid infusion rate may be about 5-10 ml/min. Alternatively, the fluid infusion rate may be about 1-5 ml/min. Alternatively, the fluid infusion rate may be about 0.5-7 ml/min. Alternatively, the fluid infusion rate may be about 0.1-2.5 ml/min. Device exit flow velocity is preferably about 60-100 cm/sec. Alternatively, device exit flow velocity is about 1-300 cm/sec.

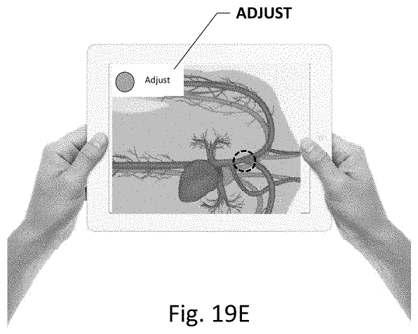

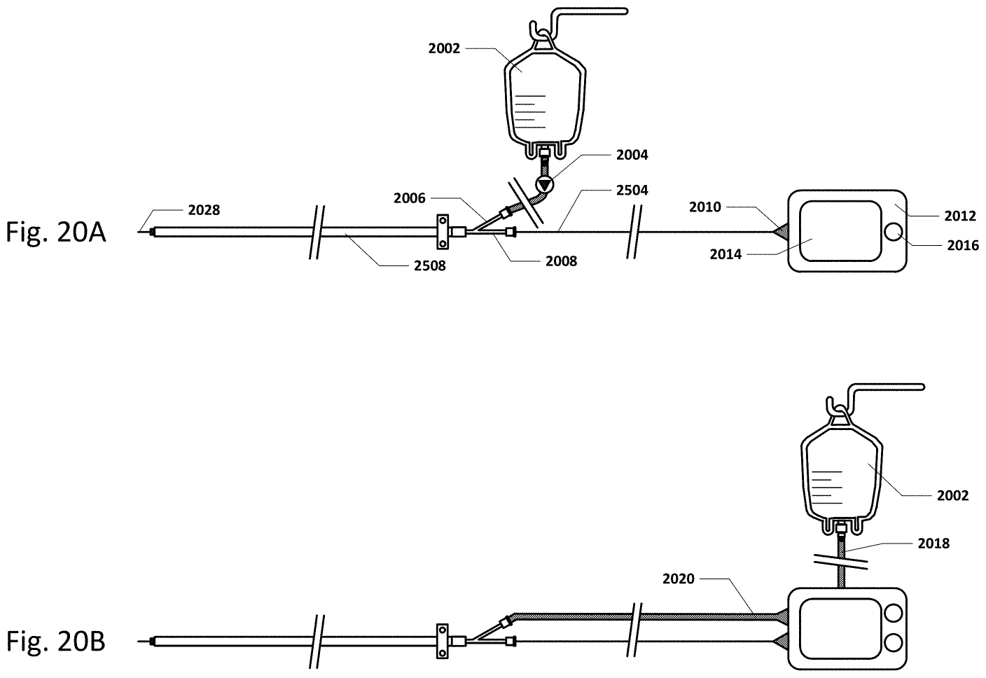

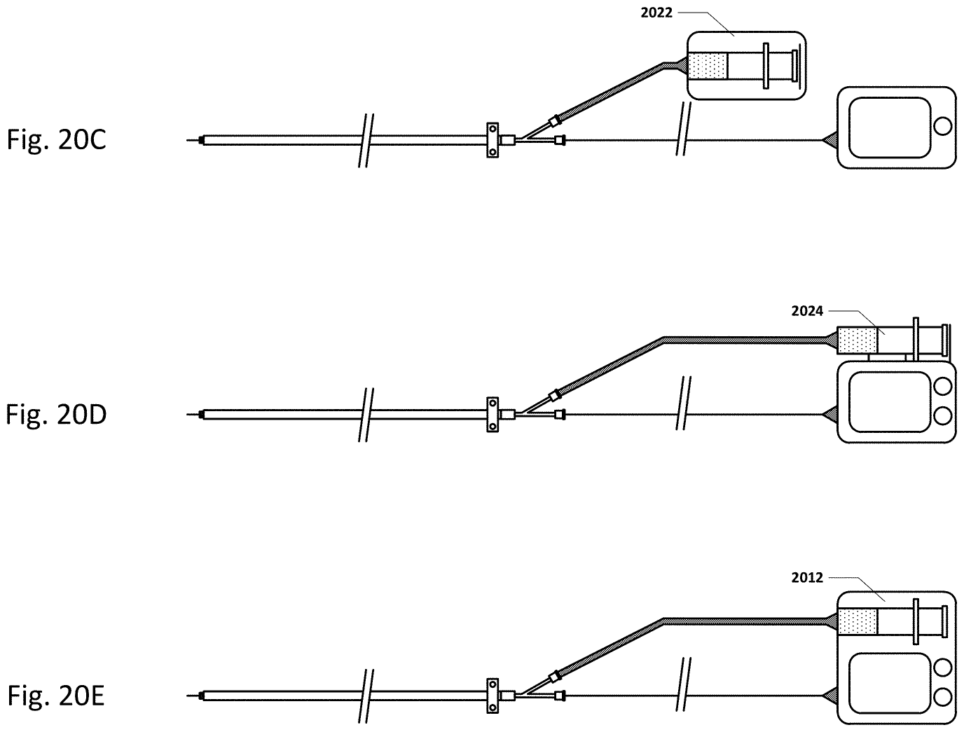

FIGS. 20A-E show some possible architectures of various embodiments of the vascular catheter navigation device. FIG. 20A shows vascular catheter 2106 and guidewire/stylet 2028, along with IV bag 2002, with optional infusion pump 2004, where the infusion bag is connected to the fluid infusion port 2806 of the vascular catheter. The guidewire/stylet is inserted/removed to/from the catheter via stylet port 2008. In some embodiments, the stylet port of the catheter may be the same port as the infusion port. Stylet/sensing connector 2010 connects to controller 2012, which may include display 2014, as well as one or more controls 2016. In this embodiment the infusion of fluid through the vascular catheter and through the flow passage(s) of the device is controlled by the IV bag/infusion pump. The IV bag may be set to a consistent drip, flow, and/or may be controlled by the infusion pump. In this embodiment, the IV bag and/or infusion pump, is connected to the vascular catheter without going through the controller. FIG. 20B shows a similar embodiment, except that the flow of IV fluid from the IV bag is controlled by the controller. IV bag 2002 is connected to the controller via IV fluid line 2018. The controller controls the infusion of fluid from the IV bag and delivers the fluid to the catheter via catheter fluid line 2020. The controller could be disposable or re-usable. The kit could also come with disposable lines which attach to the IV bag or hospital's infusion pump.

FIG. 20C shows an embodiment of the vascular catheter navigation device which includes fluid pump 2022, such as a syringe pump. The fluid pump may be a standard off the shelf fluid pump. It could be a peristaltic pump a pump driven by a lead screw, a pump driven by spring(s) such as constant force spring(s), or any suitable pump. Note that in this embodiment, the fluid pump does not connect to the controller.

FIG. 20D shows an embodiment where fluid pump 2024 connects to the controller, so that the controller can control the fluid delivery to the catheter via the fluid pump. The controller may have a module that allows the user to attach an off the shelf fluid pump, or the controller may require a specific fluid pump. The connection may be through an electrical connection, or the controller may control the infusion or fluid pump through a wireless protocol such as Bluetooth, Wi-Fi, or other. The fluid pump and/or the syringe cartridge within the fluid pump may be disposable and may be shipped sterile.

FIG. 20E shows an embodiment where the fluid pump is incorporated into controller 2012. The fluid pump and/or the syringe cartridge within the fluid pump may be disposable.

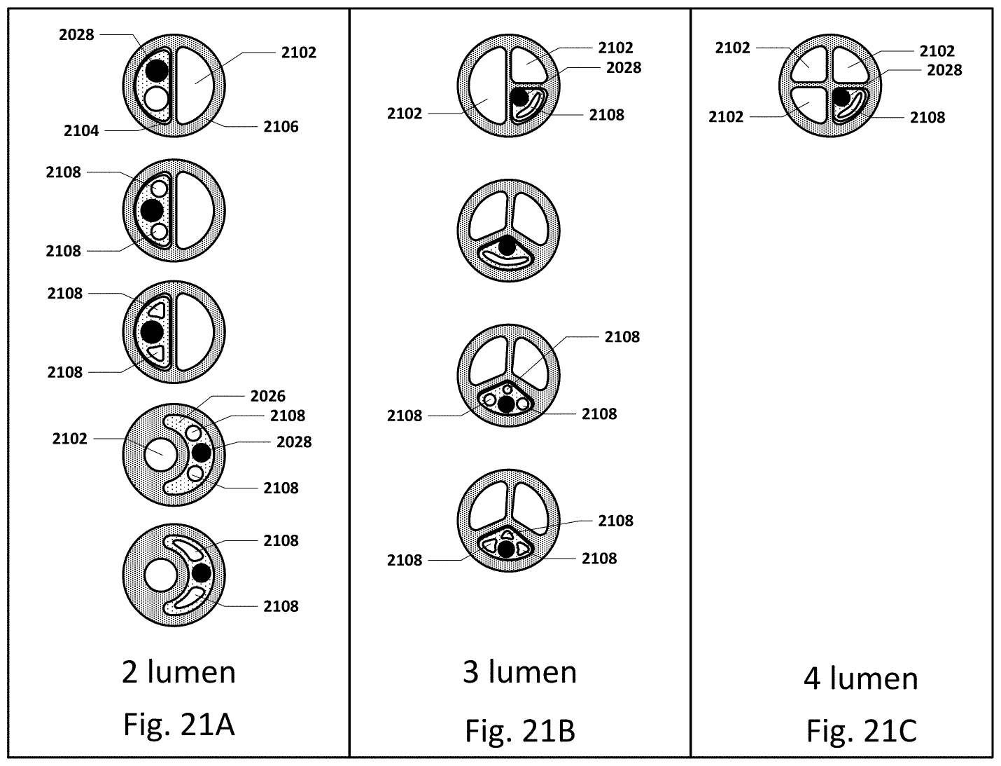

FIGS. 21A-C show some different configurations of vascular catheter lumens and variations of embodiments of the vascular catheter navigation device which work with them. Vascular catheters may have one, two, three, four, five or more lumens. FIG. 21A shows some example configurations of 2 lumen vascular catheters. These configurations include infusion lumen 2104 and auxiliary lumen 2102. The auxiliary lumen may be an additional infusion lumen, a sampling lumen, a pressure lumen, a guidewire/stylet lumen, a tools lumen, or a lumen used for any other purpose. Shown here are guidewire/stylet 2028, conduit 2026, flow passage(s) 2108 and vascular catheter 2106. Some of the various components of the vascular catheter navigation device, including the stylet, conduit, and flow passage(s) may have different cross sectional shapes to accommodate the different shape vascular catheter lumens. Some examples are shown here, but others are envisioned. The conduit may serve as a “plug” on the guidewire/stylet that blocks a lumen of the catheter to direct the flow of injected fluid from the flow passages. The shape of the conduit may be preformed, for example in the form of a polymer conduit, or may take on the shape of the lumen, for example via an inflatable or conformable conduit.

FIG. 21B shows some examples of configurations of 3 lumen vascular catheters. FIG. 21C shows an example of a configuration of a 4 lumen vascular catheter.

Although some embodiments of the vascular navigation device shown here in multi-lumen catheters show the vascular navigation device conforming to the shape of the lumen, the vascular navigation may have different cross sectional shapes, including round.

Note that although embodiments disclosed herein show the vascular catheter navigation device in an infusion lumen of a vascular catheter, it is also possible that the vascular catheter navigation device may be used in any lumen of a vascular catheter, for example a sampling lumen. It is also possible that more than one vascular catheter navigation devices may be used at once in more than one lumen.

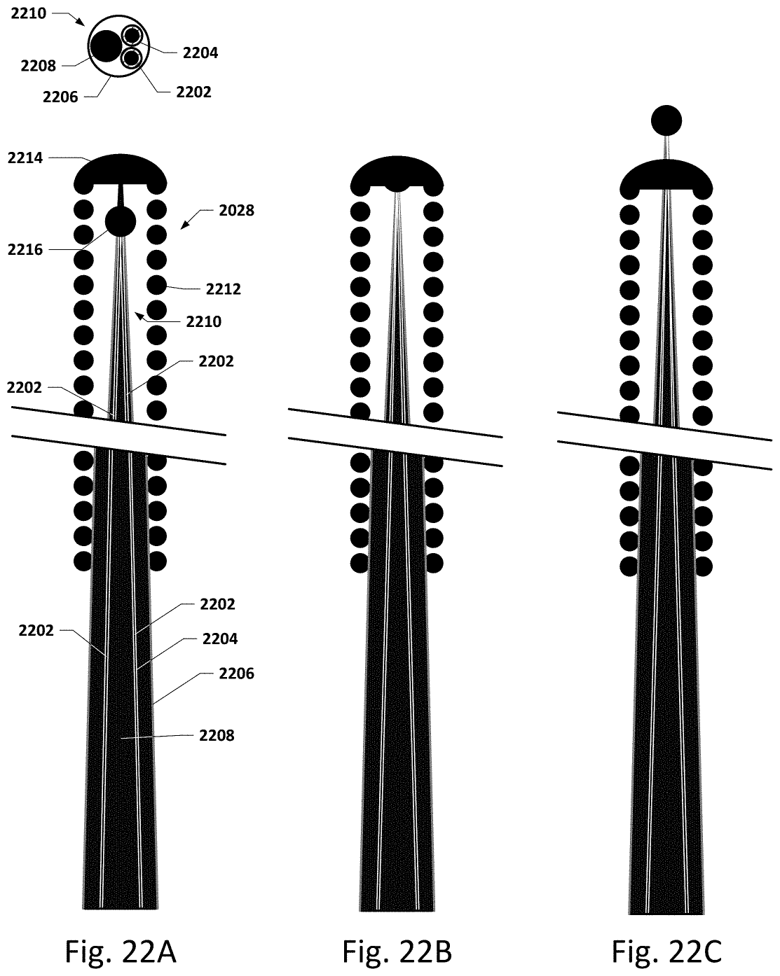

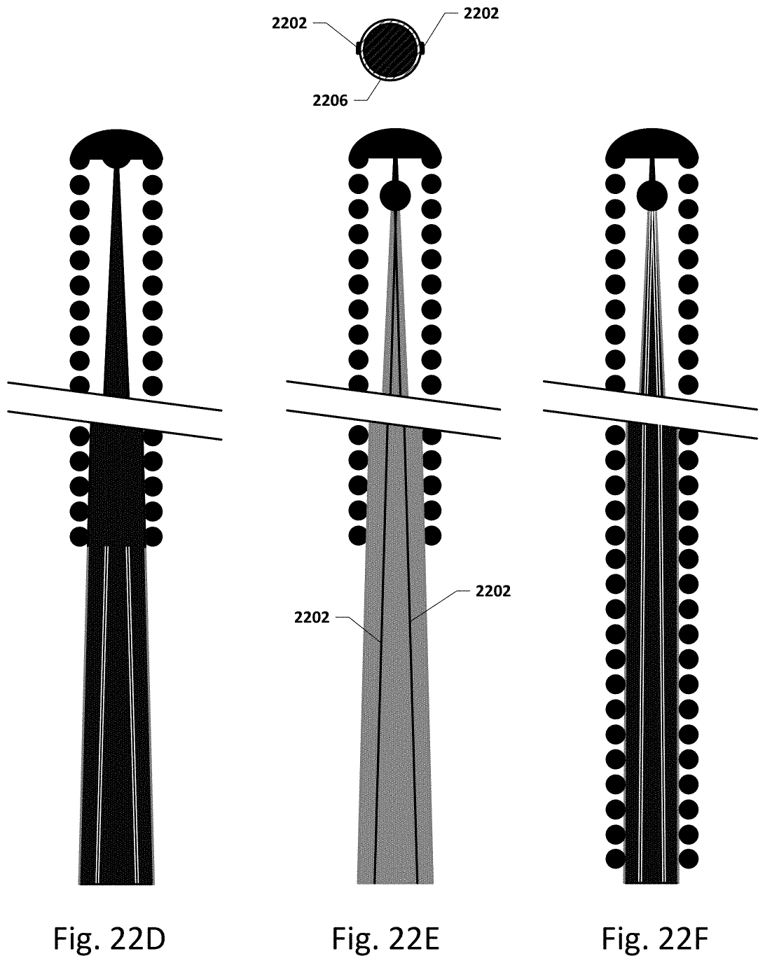

FIGS. 22A-F show various embodiments of a guidewire/stylet component of the vascular catheter navigation device. FIG. 22A shows guidewire/stylet 2028, including core 2210, coil 2212, endcap 2214 and sensor 2216. Also shown here are sensor lead wires 2202, sensor lead wire insulation layer 2204, stiffener 2208 and core enclosure 2206. The sensor lead wires connect the sensor on the distal end of the device to the controller on the proximal end of the device. There may be one, two or more lead wires. For example, a thermocouple will usually have 2 lead wires. Some thermocouples, however, may have 3 lead wires if one of the lead wires is a ground wire. The lead wires are preferably made out of metal. The lead wires may be insulated with insulation layer 2204 which surrounds each lead wire. In some embodiments, only one of the lead wires is insulated. The insulation material may be made out of polymer such as polyethylene or PTFE or polyimide or other suitable material and may or may not be heat shrinkable. The lead wire may be made out of metal, such as copper, stainless steel or other suitable material. The stiffener may be made out of metal (such as nitinol or stainless steel, etc.) and may be tapered to a smaller cross sectional dimension at the distal tip, or the stiffener may have a consistent cross section over its length. The stiffener may be round in cross sectional area or may be any other shape. The stiffener may alternatively be a polymer. The lead wire(s) may serve as the stiffener in which case, and additional stiffener will not be present.

The core, which includes the lead wire(s) and an additional stiffener, if present, may be encapsulated with enclosure 2206. Enclosure 2206 may be a tube made out of polymer, such as polyimide, polyethylene, PTFE etc., or metal or other suitable material. The enclosure may alternatively be a dip or spray coating. The enclosure may be a heat shrinkable tubing.

FIG. 22A shows a guidewire/stylet where lead wires travel to the distal end of the stylet where sensor 2216 exists separately from endcap 2214 and proximally to the endcap. FIG. 22B shows an embodiment where the endcap and the sensor are combined. FIG. 22C shows an embodiment where the sensor is distal to the endcap.

FIG. 22D shows an embodiment of the stylet/guidewire where the coil serves as the lead wire(s). In this embodiment, the lead wire(s) exit the core and are incorporated into the coil proximal to the sensor.

FIG. 22E shows an embodiment where lead(s) 2202 are made out of conductive ink. In this embodiment, the lead(s) may be on the outside of enclosure 2206. The ink may be deposited onto the enclosure. The conductive ink lead(s) may be sandwiched between two enclosures. Note that conductive ink may be used for any of the sensors, including conductance sensors, thermocouples, ECG, sensors, etc., and may be printed on the stylet and/or catheter and/or conduit, or may be printed on a flexible circuit and wrapped around, or applied to, the device.

FIG. 22F shows an embodiment of the stylet/guidewire where the coil exists over the entire length, or substantially the entire length, of the stylet/guidewire.

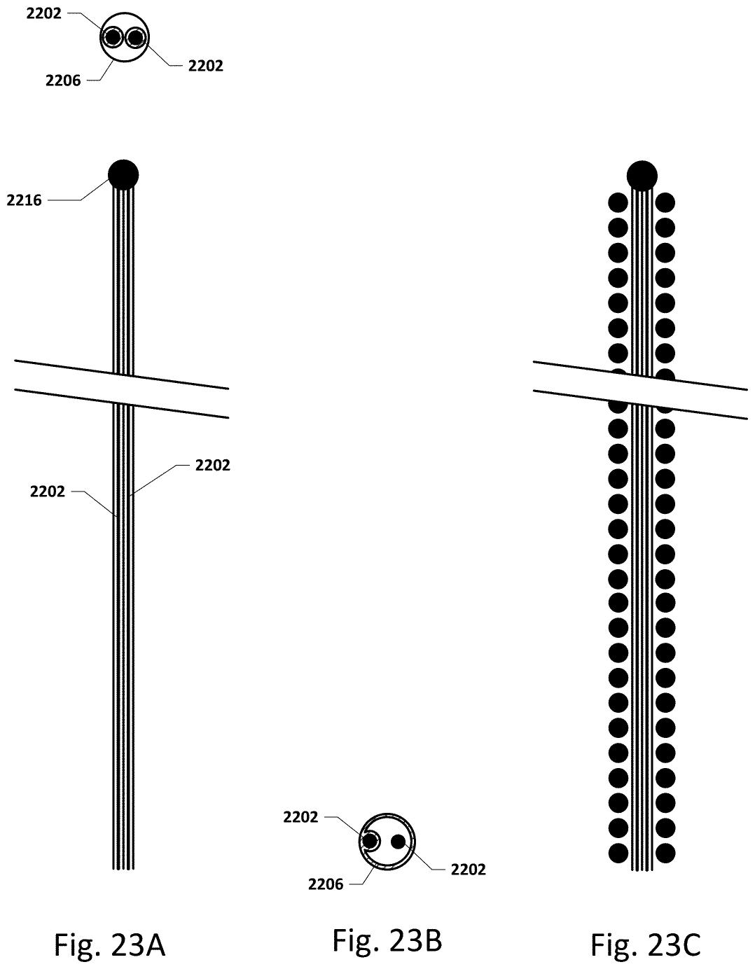

FIG. 23A shows an embodiment of the stylet/guidewire where lead(s) 2202 also serve as stiffener(s). The lead(s) may be encapsulated in enclosure 2206 and connect to sensor 2216. Additional stiffness may be added to this embodiment by using thicker leads, thicker/stiffer enclosure, for example, metal braid or coil or filament reinforced polyimide or polymer tubing etc. Alternatively, the gap between the enclosure and the leads may be filled with epoxy or adhesive. The leads may be welded or bonded to each other or to the enclosure. The enclosure may be co-extruded with one or more of the insulation layers of the lead(s), as shown in FIG. 23B. Thermoset polymers and/or metals may be used in the enclosure, insulation and/or leads. Each lead may include an insulation layer or only one lead may have an insulation layer or neither lead may include an insulation layer, for example in the embodiments where adhesive or epoxy is used to stiffen the stylet. In such embodiment, the lead diameter, cross-section design, and material may be designed to match desired stiffness of the stiffener. One or more of the leads may be spiral, coiled, or braided to achieve desired stiffener mechanical properties.

FIG. 23C shows the embodiment shown in FIG. 23A with the addition of a coil.

Any of the guidewires/styli disclosed herein may be used with any of the embodiments disclosed herein.

Where “sensor” or “sensor” is used herein, other types of sensors may be used, including any measurable parameter including temperature, opacity, light reflectivity, sound reflectivity, density, viscosity, ability to absorb light, ability to absorb sound, pressure etc.

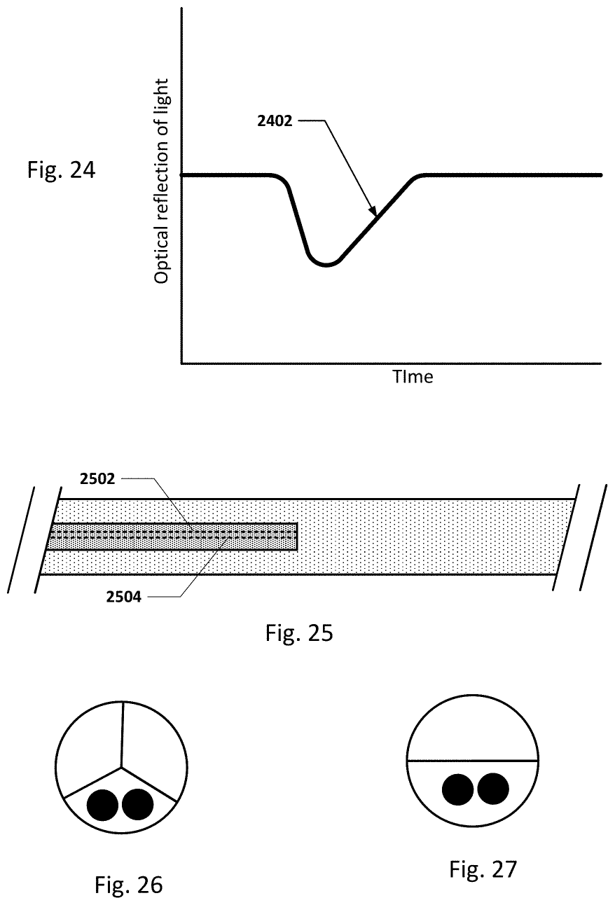

FIG. 24 shows that an optical signal can provide information on direction of blood flow and other blood flow parameters. In this embodiment, the medium is light and the parameter measured is light intensity and/or reflected light. Curve 2402 represents a measurement of reflected light over time in a blood vessel where blood flow is toward the device.

FIG. 25 shows an embodiment of a device which uses optical sensors. Fiber optic cables 2502 and 2504 can be used for transmission and detection of light. One cable may be used to introduce the medium (light) and the other cable serves as a sensor for a parameter of the medium (light intensity/wavelength). A detector and emitter combination can be used or an optical detector can be used without an emitter, requiring only one fiber. In some embodiments, light at particular wavelengths may be used. For example, red light of approximately 620 nm to 750 nm may be emitted, which is reflected more by red blood cells than by saline, or saline diluted blood. Thus a response can indicate a flow direction or characteristic. This same embodiment can be enabled more broadly with other types of visible light of about 350 nm-800 nm and near infrared light between about 400 and 1400 nm. This embodiment can be achieved with detector and/or emitters that are located at the point of measurement and potentially used in combination with a flex circuit. The optical measuring embodiment can also be used with the use of fiber optics (plastic, glass or other,) or light pipes where the actual detector and emitter are located in the controller and the light pipe or fiber optic communicates information collected at or near the catheter tip with the controller located outside the patient. This can be performed with fiber optic lines which are about 0.1 mm to about 0.5 mm in diameter or about 0.5 mm to about 4 mm in diameter. The fiber optic cable(s) may have an insulated coating. In some embodiments, a single optical fiber may be used. In some embodiments, the fiber optic cables may be cut to adjust the length of the device by cutting the device.

FIGS. 26 and 27 show a triple lumen device and a double lumen device respectively, with 2 fiber optic cables.

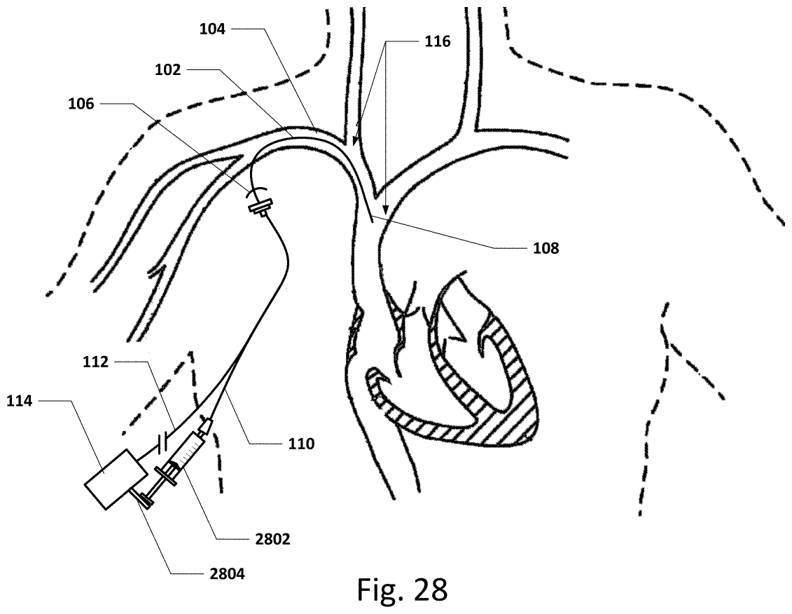

FIG. 28 shows an embodiment which includes controller 114 and a medium/infusate/injectate introduction/infusion mechanism 2802 controlled by the controller via lever or mechanism 2804. The medium introduction mechanism may be a syringe containing saline or other fluid and mechanism 2804 may be a lever controlled by a motor within the controller. Alternatively, the controller may be remote from the medium introduction mechanism, for example, the medium introduction mechanism may be automatically powered by a spring. The media introduction mechanism may alternatively be manually driven. The controller may be at the patient's bedside or remote. The controller may provide real time feedback if there are any changes of safety issues. It may be used with a standard PICC, subclavian, and intra jugular catheters, or central catheters, regardless of brand.

Controller

The controller may control delivery of the medium and receive signals from sensors which sense a medium parameter in the blood flow. In addition the controller will receive information from the one or more sensors and interpret the information to assess the location, relative location, and/or hazard zones within the vasculature, or assess other blood flow parameters. The sensor signals are communicated, via a wire, wirelessly, by fiber optic cable, or other means, back to the controller where the signal(s) are analyzed based on the measured parameter, parameter profile, parameter of more than one sensor, or change in parameter over time and/or distance. For example, the controller can determine whether the distal end of the vascular catheter navigation device is in an artery instead of a vein, based on magnitude and direction of blood flow, and/or other flow parameters, near the vascular catheter navigation device. For example, if the controller determines that the distal end of the vascular catheter navigation device is in an artery instead of a vein, a specific identifying signal may sound, including an audible, visual signal etc., instructing the user to remove the vascular catheter navigation device, and any other device, such as sheaths, catheters etc., and apply pressure to the blood vessel. For example, instructions for advancing, retreating, redirecting, stopping movement of, or removing, the vascular catheter navigation device may be displayed by the controller on a screen connected to the controller. The connection may be wired or wireless and the screen may be local or remote. The signal from the controller may be transmitted over Bluetooth, or other wireless protocol, to a computer such as a laptop, tablet, phone, watch, or other peripheral device.

The controller may control introduction of medium, including injection of a temperature controlled solution, such as saline, introduction of sound, introduction of light, introduction of a fluid containing a level of a parameter, etc. Temperature controlled may mean a temperature which is different than body temperature.

Injection Mechanism and Fluid Properties

The infusion drip, bolus, droplet, stream, etc., used to detect catheter location may have specific parameters. The infusion may be a drip or it may be a stream. The preferred intermittent volume size (drip, drop, bolus, intermittent stream) is between about 0.5 cc to about 3 cc, but can range between about 0.1 cc and about 10 cc. Alternatively, the volume may range from about 0.5 cc to about 1 cc. Alternatively the volume may range from about 0.1 cc to about 2.5 cc.

The preferred drip interval may be between about every 0.5 second to about every 4 seconds to a broader range of about every 0.25 seconds to about every 10 seconds. Where the infusion is a continuous stream, the preferred flow rate is about 4 cc/minute but may range from about 0.25 cc/minute to about 15 cc/minute or from about 0.1 cc/minute to about 30 cc/minute or from about 0.1 cc/minute to about 60 cc/minute.

The pressure applied to the injection mechanism (syringe, for example) for injection may be around 3 psi but may range from about 1 psi to about 20 psi, or the range may be from about 0.1 psi to around 200 psi.

The controller may control an injection device, or volumetric displacing device, such as a syringe, so that the injection device introduces a controlled volume and/or rate of fluid into the catheter or stylet/guidewire. The fixed volume and/or rate of fluid may be at a controlled temperature, either above or below that of blood (approximately 37 degrees Celsius), or at a known temperature which is measured. The injection device may inject a controlled volume and/or rate of fluid at predetermined intervals, or other intervals, or continuously. The controlled volume and/or rate of fluid injection may remain the same throughout a procedure, or the volume and/or rate may change depending on the patient, the location of the catheter/system within the vasculature, etc. For example, the volume and/or rate of fluid injected may increase as the tip of the catheter gets closer to the heart. The volume and/or rate may be different for different sized vascular catheters or different sized lumens of vascular catheters, for example in catheters with multiple lumens.

The volume and/or rate of fluid injected may be controlled by a lead screw, cam, linear actuator motor, peristaltic pump, spring(s) etc. The force of the injection requirements may also be controlled and/or monitored. For example, if an unusually high force is required to inject the fluid, an alert may tell the user that a possible catheter blockage situation exists, including a catheter kink, a blood clot, the catheter tip up against a vessel wall, or within a small vessel, or other catheter patency situation. Higher or lower force injections may be used in different areas of the anatomy, or to confirm location within the anatomy. For example, a higher forced injection of a smaller volume and/or rate may provide different temperature curve information than a lower force, higher volume and/or rate injection. Small volume injections at a higher frequency may provide different information than larger volume injections at a lower frequency, etc.

The fluid injector may also be configured to withdraw fluid through the catheter/stylet/guidewire to determine injection lumen/tip patency. The controller may assess force to withdraw fluid to determine that fluid is flowing freely through the catheter/stylet/guidewire. If fluid is not flowing freely, a patency alert may alert the user. Alternatively the controller may have a sensor which senses the existence of blood in the system when the injector withdraws fluid through the catheter/stylet/guidewire. This may be done optically or otherwise.

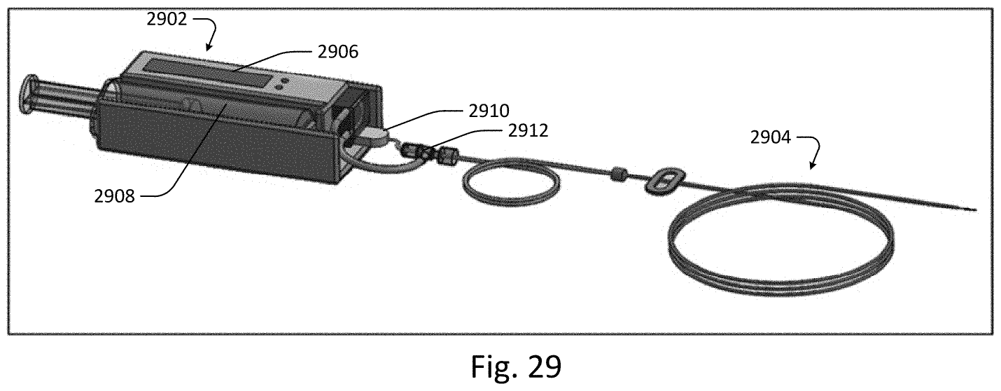

An embodiment of the injection mechanism is shown in FIG. 29. Shown here is injection mechanism 2902, which may be part of, or separate from, the controller, sensing catheter or stylet 2904, display 2906, syringe 2908 (which may contain pre-loaded sterile fluid, such as D5 W, and may be packaged with the infusion device), electronics connector 2910, which connects to the sensors, and fluid connector 2912, which is in fluid communication with the fluid exit ports on the device. The injection mechanism, which may be controlled by the controller, provides precise, low-flow infusion of the fluid via a constant force spring or controlled motor. Display 2906 may provide visual feedback of the device tip location in the anatomy, providing real-time navigation guidance within the anatomy. This embodiment may include an automated injection system for the cartridge/syringe/reservoir which may be a motor driving lead screw or one or two or more springs. The controller may control the infusion delivery parameters, including pressure, volume, frequency, rate, etc. The controller may also control the GUI/display. The buttons, shown in FIG. 29 may turn the device on and off, purge the catheter of air prior to insertion, and/or may stop operation of the device in case of a sensed problem situation. The unit may be fully disposable, partially disposable or non-disposable, and may reside in the sterile field or the unsterile field during the procedure. The fluid loaded syringe may be packaged and sterilized with the stylet/catheter.

The system may come packaged with a prefilled injection device, or a fillable injection device. Saline may be used as the fluid, or D5 W, or other suitable fluid. Contrast medium may be used (which is a higher viscosity than saline). Fluids of differing viscosity may be used, or fluids may be mixed (such as contrast medium and saline) to achieve a desired viscosity or other desired properties. Fluids of different surface tension, different specific heat capacity, different acidity or other different attributes may be used. Fluids with properties that differ from those of blood will provide different sensed parameter, curves and therefor provide different information regarding the location of the catheter/guidewire/stylet tip in the vasculature. Some fluids may be soluble in blood and others less soluble. Since the injection fluid is injected into the blood stream, the fluid used will preferably be biocompatible.

Additives may be added to the injection fluid for different results. For example, salts, such as NaCl may be added. Different salts or other additives may improve an ECG signal in embodiments that include an ECG electrode. A different fluid (liquid or gas) may be introduced with the primary fluid to modify the fluid properties. For example, a biocompatible liquid or gas may be “bubbled” into saline.

More than one injection fluid may be used, either mixed and injected through the same lumen and exit port(s), or injected separately, through different lumens and/or different exit ports. One or more of the injection fluids may include a drug or medication.

A user interface controlled by the controller may include a display, alerts (auditory, visible, lights, vibrations etc.) and other information. The user interface may include a display of the anatomy with a virtual reality indicator of the location of the catheter/guidewire/stylet tip within the anatomy. For example, the display may be an image of the human vascular system, and a moving indicator, such as a light, may show where within the anatomy the catheter/guidewire/stylet tip is located. The display may be actual size, and possibly even projected upon the patient, or it may be a smaller or larger size, for example, displayed on the controller, a tablet, or projected up on the wall. The controller and/or display may include a computer, laptop, tablet, mobile phone, virtual reality/augmented reality glasses, etc.

The system may be fully disposable. A fully disposable system primary package includes: syringe, syringe pump, the syringe (or a sterile vial) filled with the fluid of choice, a controller, a user interface which can exist as any combination of display, alert, and lights, catheter, stylet/guidewire, and introduction mechanism. All, or some, of these elements may be fully disposable. For example, the controller and user interface/display may not be disposable, but the navigation device, syringe, syringe pump may be. By doing so, the chance of infection will be reduced.

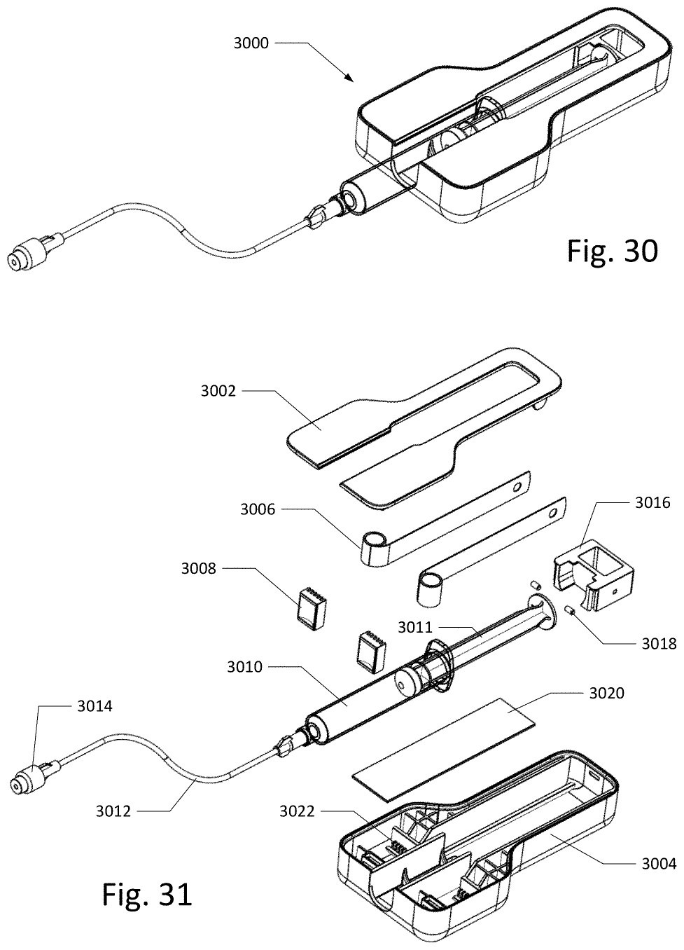

FIG. 30 shows a version of injection mechanism, or fluid pump, 3000 which may be disposable.

FIG. 31 shows an exploded view of the injection mechanism shown in FIG. 30. Shown here are cover 3002, base 3004, springs 3006, spring adjusters 3008, syringe 3010, infusion tubing 3012, valve 3014, carriage 3016, carriage pins 3018, grip tape 3020 and base spring adjuster receptacles 3022. Springs 3006 may be constant force springs. One, two, or more springs may be used. Different spring sizes, applying different forces, may be used. To adjust for different spring sizes, spring adjusters 3008 may be moved within base spring adjuster receptacles 3022 to accommodate larger or smaller springs within the injection mechanism housing. Carriage 3016 engages with the back end of syringe plunger 3011 and slides within the injection mechanism as fluid is injected. The carriage may start in the “cocked” position (where the syringe plunger is pulled back and the syringe body contains fluid). The springs apply a force to the carriage, which in turn applies a force to the syringe plunger, which forces fluid through infusion tubing 3012 and into the sensing stylet or catheter. Valve 3014 may be present to initially prevent fluid from flow through the infusion tubing before the navigation device is connected to the infusion tubing, and possibly during sterilization and shipping. The valve may open only when it is connected to the sensing stylet or catheter, allowing pressurized fluid to flow through the stylet/catheter.

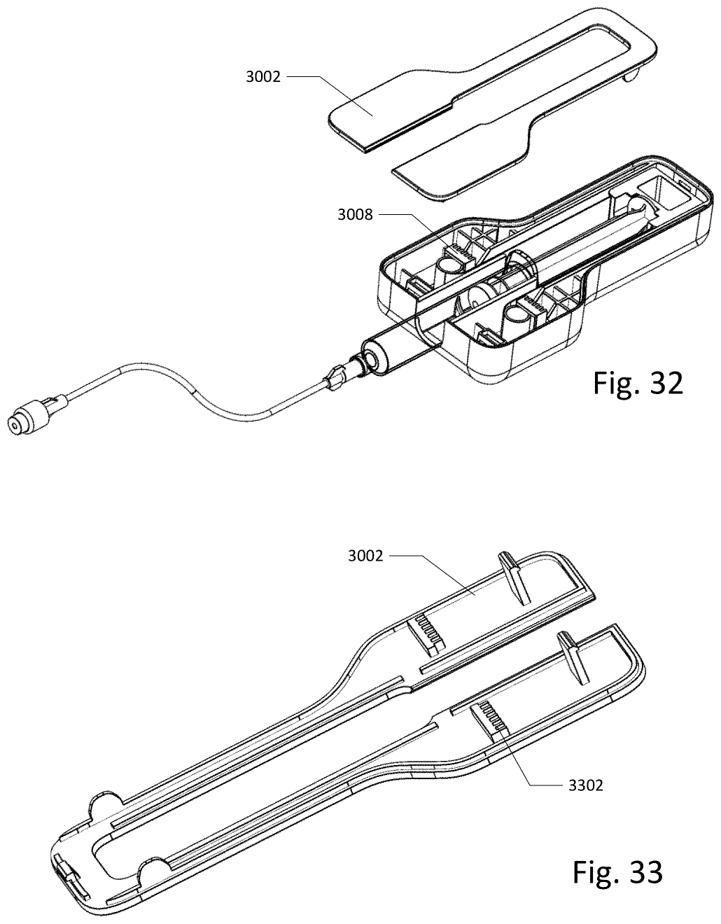

FIG. 32 shows the embodiment of the injection mechanism shown in FIG. 30 where cover 3002 is removed, revealing the other components in place within the housing. Spring adjusters 3008 are shown here engaged with the base spring adjuster receptacles (not visible). The spring adjusters may be moved back and forth and locked in place via notches to accommodate springs of different sizes and forces.

FIG. 33 shows cover spring adjuster receptacles 3302 on the underside of cover 3002. These perform the same duty as the base spring adjuster receptacles, helping to lock the spring adjusters in place when the cover is connected to the base.

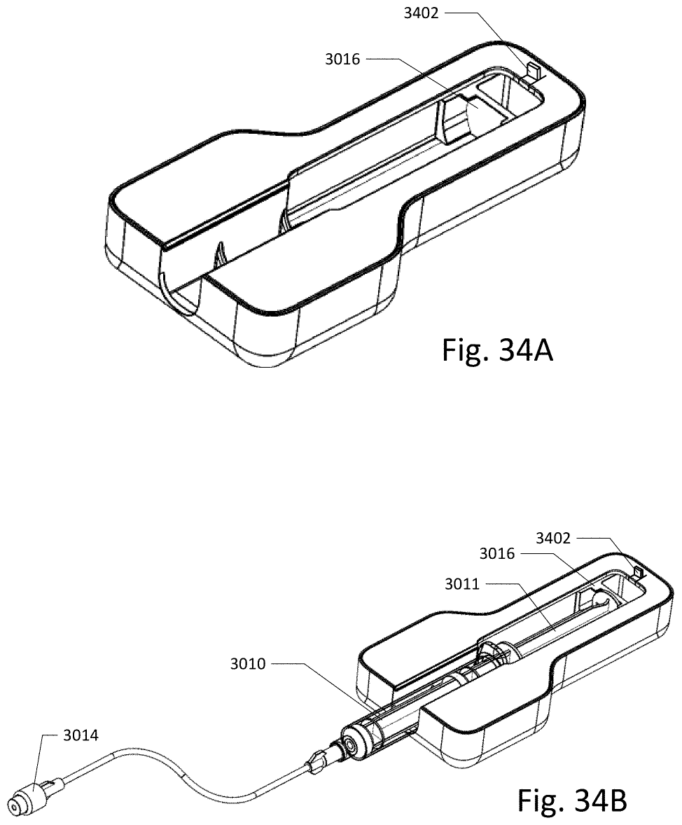

FIGS. 34A and 34B show another embodiment of the injection mechanism. This embodiment includes locking mechanism 3402 to engage with carriage 3016. The locking mechanism is an alternative, or addition, to valve 3014, which may be used to lock carriage 3016 in the “cocked” position so that fluid from the syringe does not escape the system before the sensing stylet/catheter is connected to the injection mechanism. Releasing locking mechanism 3402 allows carriage 3016 to move within the housing and force syringe plunger 3011 to apply pressure to the fluid within syringe 3010 and through the infusion tubing. If both valve and locking mechanism are present, then the locking mechanism is released after the stylet/catheter is connected to the injection mechanism.

In some embodiments, the syringe is loaded into the injection mechanism before the procedure. In other words, the fluid filled syringe is not packaged with the injection mechanism. In this case, the carriage may be pulled back and locked into the locking mechanism, the syringe may be loaded, and the injection mechanism connected to the sensing stylet/catheter. At this point, the locking mechanism may be released to allow fluid to flow through the sensing stylet/catheter.

In some embodiments, the fluid pump is designed in such a way that the syringe may be loaded into the pump using only one hand. For example, the pump may be locked in the “cocked” position first, using one hand, and then the syringe may be loaded into the pump with one hand, and the pump may be unlocked to allow the infusion fluid to flow through the navigation device, using only one hand. In some embodiments, the inserting of the syringe and the locking of the pump into the “cocked” position may be done in a single movement, and in some embodiments, with one hand.

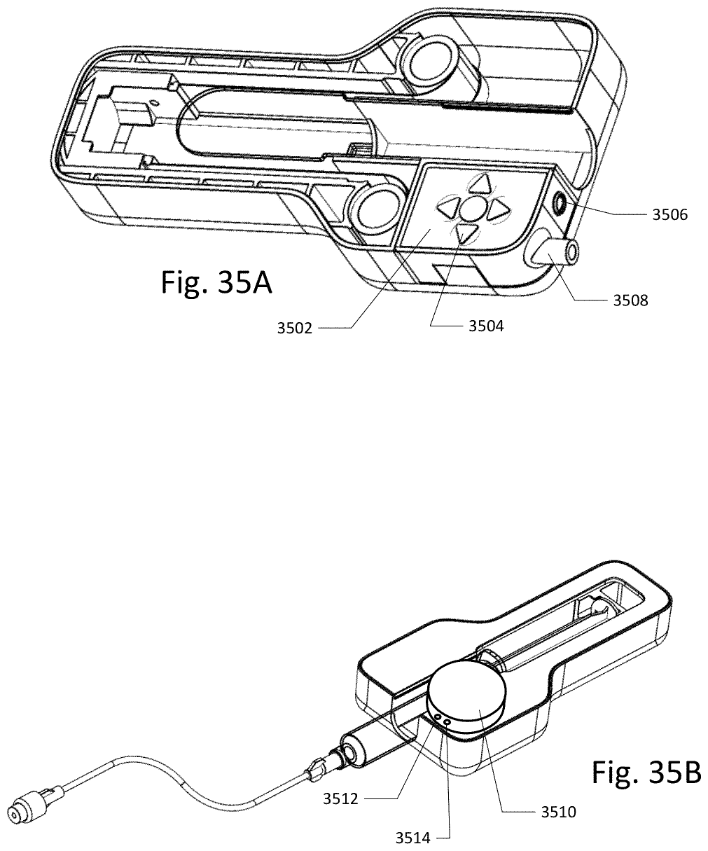

FIGS. 35A and 35B show an embodiment of an injection mechanism which includes an add-on navigational component and/or controller component. Component 3502 shown in FIG. 35A may include display/control interface 3504 as well as fluid connector 3508 and electrical connector 3506. Component 3502 may be incorporated into the injection mechanism during sterilization and package, or may be added after unpackaging the injection mechanism, before the procedure. Component 3502 may snap into place in the housing of the injection mechanism. This allows the control and feedback of the sensing stylet/catheter to be in one place. The injection mechanism may be placed on, or secured to, the patient, bed etc.

FIG. 35B shows component 3510 with fluid connector 3512 and electrical connector 3514. Component 3510 may snap on top of the injection mechanism as shown here. The add-on component may be added by piercing a sterile barrier around the injection mechanism/fluid pump.

In some embodiments, the fluid pump may incorporate catheter and/or stylet and/or cable management, to minimize clutter on and around the patient.

Another embodiment includes all of the items listed above where the display is non-disposable. The display may be within the non-sterile field and communicate via cable or a wireless communications protocol such as Bluetooth. Alternatively, the display may be within the sterile field using a wired or wireless connection. Additionally/alternatively, the display may be projected on glasses—either virtual reality or augmented reality glasses. The glasses may be within the sterile or non-sterile field. Additionally, a projector may project the display on a surface of choice and the projector may be in sterile or non-sterile field.

Another embodiment consists of two subsystems. The disposable elements may include catheter, stylet/guidewire, and a fluid filled volume displacing device, such as a syringe. The non-disposable elements may include a controller in a housing, mechanics/motors to depress the lead screw on the syringe/cartridge, display, audio, and visual elements, as well as user interaction buttons, etc.

Any of the catheter/stylet/guidewire placement and/or patency techniques disclosed herein may be used while placing the device in the vasculature, as well as after placement, to monitor the device during use, and to determine that the device has not significantly strayed from its placement location over time. This time frame may be hours, days, weeks or months.

Any of the embodiments disclosed herein may be used with any type of central vascular catheter including central venous lines, clavicle lines, midline, etc. In addition, any of the embodiments disclosed herein may be used with peripheral vascular catheters, dialysis catheters, and cardiac catheters including catheters used for: coronary arteries, patent foramen ovale, atrial septal defect, etc. Any of the embodiments disclosed herein may be used with any type of urinary catheters. Similar technology may be used in underwater navigation, mining, oil and gas exportation, utility fabrication or repair, transportation infrastructure fabrication and repair, etc.

Other technologies may also be used in conjunction with the sensor readings from the vascular catheter. For example ECG readings, ultrasound readings, Doppler readings, x-ray readings, inductive current technology, pressure readings, etc. Some, all or no readings may be augmented via a turbulence inducer. These, and other, other types of readings may be used in conjunction with the sensor readings by the controller to determine the location of the vascular catheter navigation device distal tip. Specific modalities may be better at identifying specific vascular landmarks or conditions.

For example, any of the conductive components of the vascular navigation device may be used as an ECG lead. Another ECG lead may be placed on the patient's skin or may be incorporated into the navigation device. For example, the guidewire stylet stiffener, coil, enclosure, thermocouple leads, sensor leads, thermocouple, endcap, conduit, etc. may be used as an ECG lead or leads. Alternatively, a separate ECG lead may be added to the system.

Example of Data Processing System

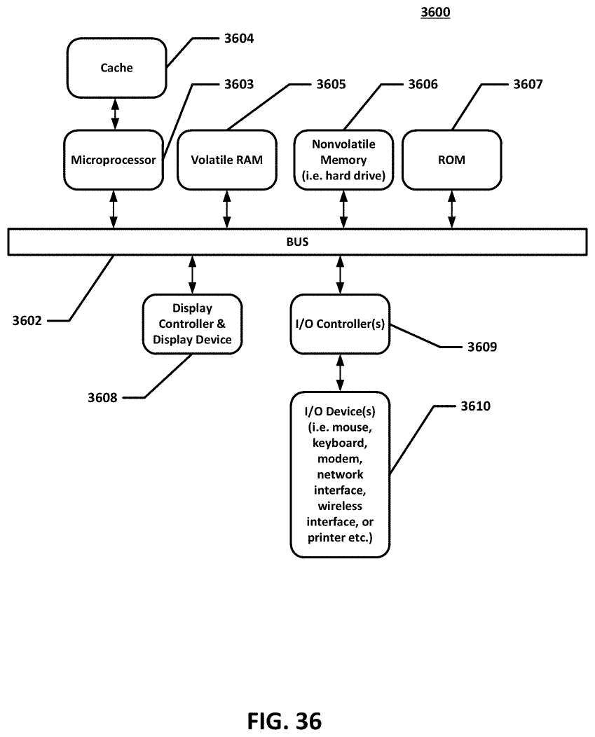

FIG. 36 is a block diagram of a data processing system, which may be used with any embodiment of the invention. For example, the system 3600 may be used as part of the controller. Note that while FIG. 36 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 36, the computer system 3600, which is a form of a data processing system, includes a bus or interconnect 3602 which is coupled to one or more microprocessors 3603 and a ROM 3607, a volatile RAM 3605, and a non-volatile memory 3606. The microprocessor 3603 is coupled to cache memory 3604. The bus 3602 interconnects these various components together and also interconnects these components 3603, 3607, 3605, and 3606 to a display controller and display device 3608, as well as to input/output (I/O) devices 3610, which may be mice, keyboards, modems, network interfaces, printers, and other devices which are well-known in the art.

Typically, the input/output devices 3610 are coupled to the system through input/output controllers 3609. The volatile RAM 3605 is typically implemented as dynamic RAM (DRAM) which requires power continuously in order to refresh or maintain the data in the memory. The non-volatile memory 3606 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 36 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 3602 may include one or more buses connected to each other through various bridges, controllers, and/or adapters, as is well-known in the art. In one embodiment, the I/O controller 3609 includes a USB (Universal Serial Bus) adapter for controlling USB peripherals. Alternatively, I/O controller 3609 may include IEEE-1394 adapter, also known as FireWire adapter, for controlling FireWire devices, SPI (serial peripheral interface), I2 C (inter-integrated circuit) or UART (universal asynchronous receiver/transmitter), or any other suitable technology. Wireless communication protocols may include Wi-Fi, Bluetooth, ZigBee, near-field, cellular and other protocols.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the Figs can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the preceding Figs may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

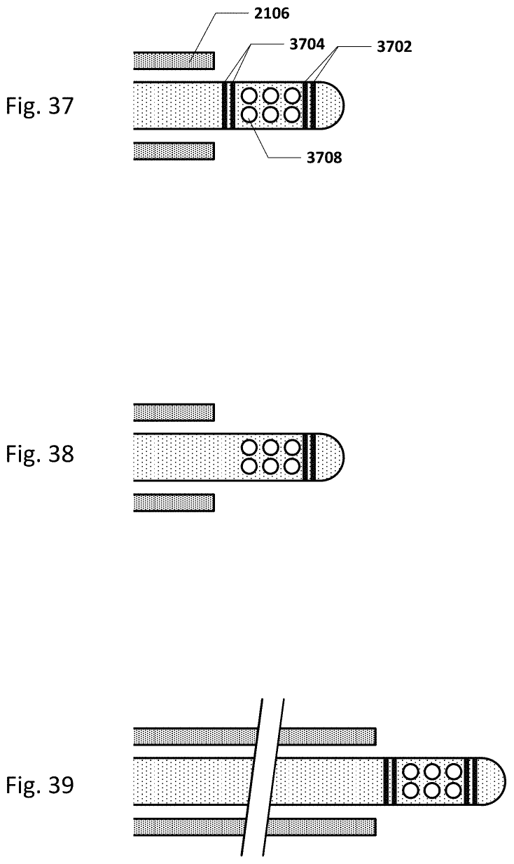

FIG. 37 shows an embodiment of the vascular catheter navigation device which includes sensors, or electrodes, for measuring conductivity. Shown here are distal electrode pair 3702, proximal electrode pair 3704, fluid exit point 3708, and catheter 2106. A single sensor may comprise a pair of electrodes. For example, distal electrode pair 3702 may represent a distal sensor and proximal electrode pair 3704 may represent a proximal sensor. Electrodes may be on the surface of the device, or may be solid discs in some embodiments. In some embodiments, fluid exit point 3708 may include multiple small openings. The fluid exit point may be forward facing as shown in some embodiments herein, or side/laterally facing, as shown here. The multiple small openings allow for a more diffuse infusion of medium fluid, as opposed to a more directed stream which may occur with a single opening. Multiple openings may run essentially circumferentially (360 degrees) around the device or may exist less than essentially 360 degrees around the device. FIG. 37 shows an embodiment with 2 pairs of electrodes and a diffuse exit port. FIG. 38 shows an embodiment with only one pair of electrodes. In some embodiments, the sensor, or electrode pair, is close (between 0.05 mm and 1 mm) to the fluid exit port/holes as this increases the sensitivity of the sensor.

In embodiments which use conductivity or impedance to determine the location of the vascular navigation device, a current is applied to an electrode of a sensor and the conductivity or impedance of the blood/medium mixture between two electrodes is sensed by the second electrode of a sensor. The driving frequency of the signal may be around 10,000 Hz. Alternatively the driving frequency may be around 500 Hz to around 100,000 Hz. Alternatively the driving frequency may be higher than around 100,000 Hz. The sampling frequency may be around 50 Hz. Alternatively, the sampling frequency may be around 25 Hz to around 100 Hz. The sampling frequency may be fixed, or may be variable and may depend on the frequencies within the detected conductivity or impedance signal.

FIG. 39 shows an embodiment where the medium infusion lumen runs the length of the guidewire/stylet. The infusion lumen of any of the embodiments disclosed herein may run the length of the guidewire/stylet/catheter.

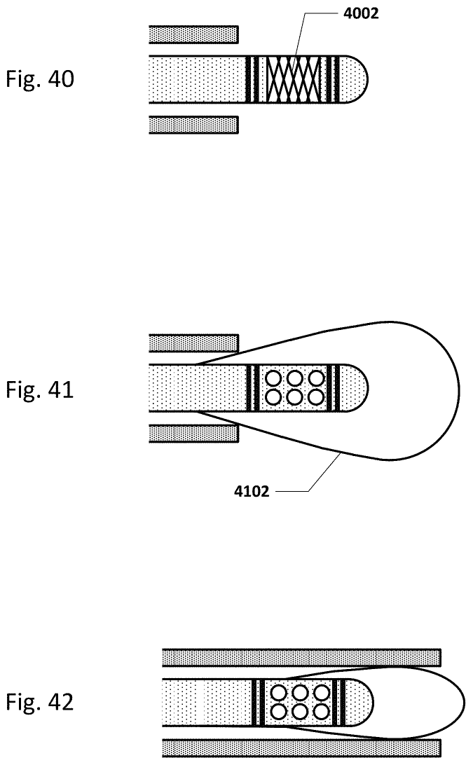

FIG. 40 shows an embodiment of the vascular catheter navigation device which includes open mesh or braid 2102 as a component of the fluid exit point. The mesh/braid may encompass essentially 360 degrees of the device or may encompass less than essentially 360 degrees of the device. The mesh/braid may be metal, polymer or other suitable material.

FIGS. 41 and 42 show an embodiment which includes spacer 4102 which maintains a space between the vessel wall and the electrode sensor(s) so that the sensors don't directly touch the vessel wall. The spacer may be a simple wire loop as shown here, or may have 2 or more loops, like a whisk, or may be of other suitable configurations. Preferably, the spacer may be compressed for introduction into and removal from the vessel. For example, the spacer may be compressed by pulling the guidewire/stylet into the catheter as shown in FIG. 42.

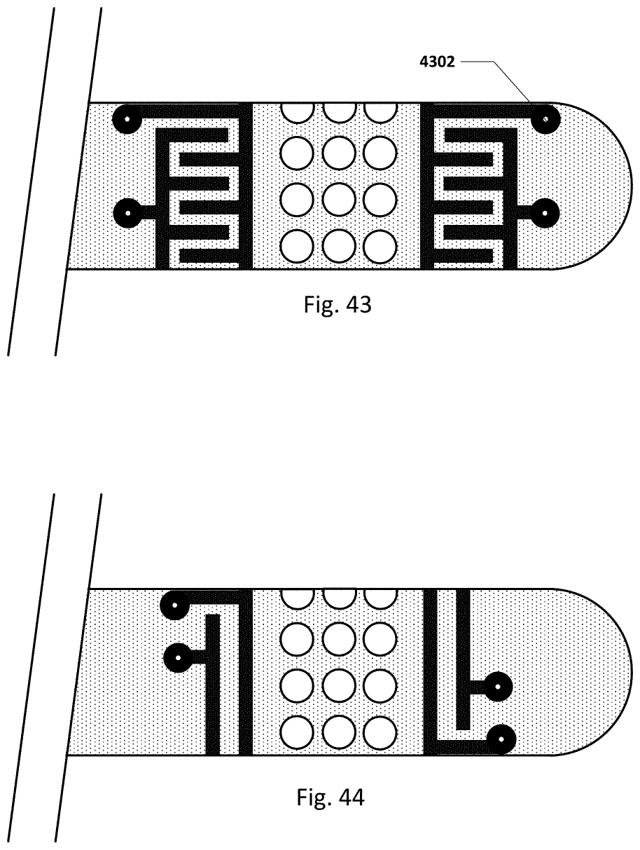

FIGS. 43 and 44 show some possible embodiments of the electrode pairs at the distal end of the device. A serpentine or other suitable pattern is shown in FIG. 43 to increase the surface area of the two electrodes in an electrode pair. The distance between the electrodes within an electrode pair may also be altered to optimize the signal. Connectors 4302 are used to connect the electrodes to wires/leads, or other conduction mechanisms within the device, back to the controller. For example, connectors 4302 may connect to a wire underlying the electrodes which runs the length of the device back to the controller. Alternatively, connectors 4302 may connect to tracings on the inside or the outside of the device, or leads embedded in the wall of the device.

Disclosed herein are various embodiments of the vascular catheter navigation device which rely on injecting an injectate, or medium, into the blood stream, where the injectate has a parameter, the value of which differs from that of blood. For example, the injectate may have a different temperature or conductance or impedance than the temperature, conductance or impedance of blood. Because these embodiments are sensing and analyzing the injectate parameter to determine the flow characteristics of the blood within a vessel, it may be important that the flow characteristics of the injectate be repeatable and meaningful. Different injectate exit port designs result in different injectate flow characteristics, and impact the data collected by and analyzed by the controller.

It is also desirable that the injectate flow in proximity to the sensors on the device so that the sensors can measure the changes in the sensed parameter. It may be desirable for the injectate flow to surround the navigation device essentially 360 degrees or as close to 360 degrees as possible, particularly in laminar, or less turbulent blood flow.

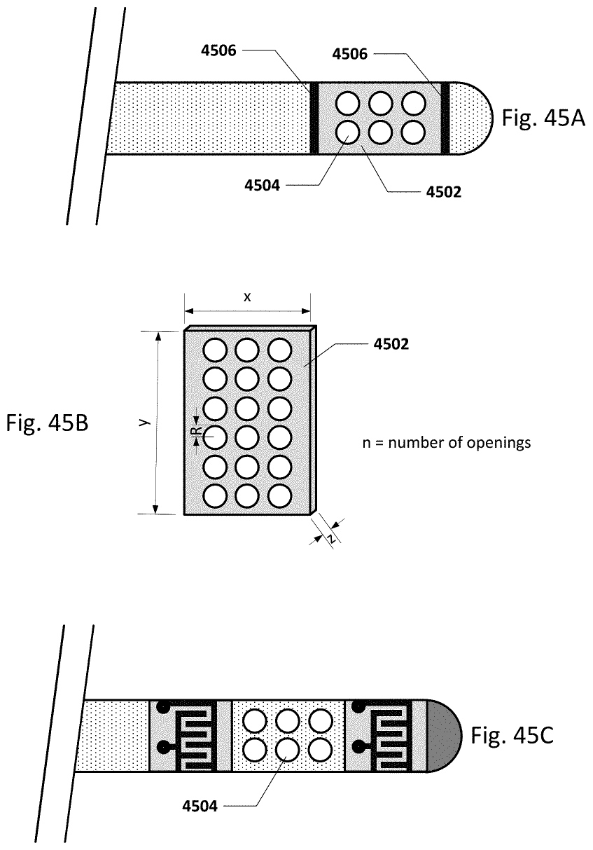

To achieve this, some embodiments include an injectate exit or port which diffuses the exit flow of injectate, to control and/or minimize the injectate exit velocity. These types of injectate port exit designs are termed “diffuse” exit port designs. Such a diffusing exit port may incorporate multiple openings or a mesh, similar to those shown in FIGS. 37-44. To further illustrate this type of fluid exit point or port, please refer to FIGS. 45A-C.

FIG. 45A shows the distal end of a vascular catheter navigation device with diffuse exit port area 4502. The exit port includes openings 4504 and in this embodiment, is between two sensors 4506, each of which may be a pair of electrodes. The exit port 4502 shown in this embodiment is manufactured by wrapping a thin perforated sheet around an opening in the device, creating the diffuse exit port. An example of the sheet is shown in FIG. 45B. The sheet may be made from polyimide or any other suitable thin and strong material, such as a polymer, a metal, etc.

FIG. 45C shows an embodiment where openings 4504 are incorporated into the surface of a device. The device may run through essentially the entire length of the catheter inner lumen or a portion of the catheter inner lumen. For example, the vascular access device may include an elongated polyimide, or other material, tube with openings 4504 cut into the wall of the tube near the distal end of the tube. Sensors may be sheets which are wrapped around the tube as shown here, or may be other types of sensors as disclosed herein. The sensor leads (not shown) may be wires or tracings on either the inside or outside of the tube, or may be embedded within the wall of the tube.

By showing the flattened diffuse exit port we can see that the exit port has a surface area, X times Y. It also has a percentage of opening area which is the surface area of the sheet minus the total surface area of the openings. If the openings are circular, the area of each opening is πR². If the openings are circular, then the total surface area of the openings is n times πR². The sheet shown here also has a thickness shown as z. in some embodiments, the resistance to exit port flow can also be increased to minimize and/or control the injectate exit port velocity. Flow resistance can be increased by increasing the number of openings (for a given open area) and/or increasing the perimeter of the openings (for a given open area), and/or increasing the thickness (z) of the openings (for a given open area). For example, a mesh with small openings will have a higher resistance to flow than a single opening of the same opening area.

A diffuse exit port design may include multiple holes in the device, holes in a secondary material that is attached the device, a mesh (polymer, metal, etc.) that is incorporated into the device, a break, or opening, in the device which is supported by other structures, such as a core wire, a sponge (Polymer, sintered or 3D printed metal or polymer), etc.

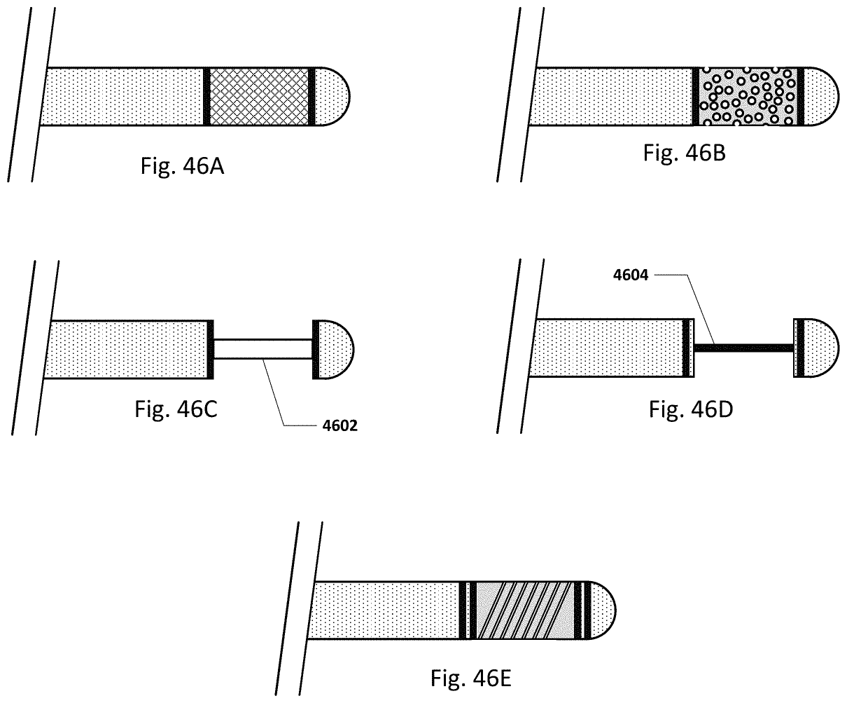

FIGS. 46A-D show some embodiments of diffuse exit port designs including a mesh (FIG. 46A), a porous polymer (FIG. 46B), a large exit port opening spanned by struts 4602 (FIG. 46C), a large exit port opening spanned by central core wire 4604 (FIG. 46D) and a spiral exit port opening (FIG. 46E). The materials of the exit port areas may be rigid, flexible, or semi-rigid.

Exit port design and distance from port to sensors are variables in the design and are optimized such that the sensor functions well in a wide range of conditions, including a range of blood flow velocities (1 cm/sec-200 cm/sec in either direction), a wide range of vessel and organ diameters, both small and large (0.5 mm diameter to 100 mm in diameter), and a wide range of infusion flow rates of the injectate (0.001 cc/min to 100 cc/minute, or more optimally 0.5 cc/min-5 cc/min), etc.

It may be desirable in some embodiments to limit the velocity of the injectate as it exits the injectate exit port. This can be done by limiting the injectate rate on the proximal end of the device, and can also be done by increasing the area of the opening(s) of the injectate exit port. Increasing the area of the openings of the exit port can be done by increasing the percentage of area that is open within the injectate exit port, and/or increasing the surface area of the injectate exit port.

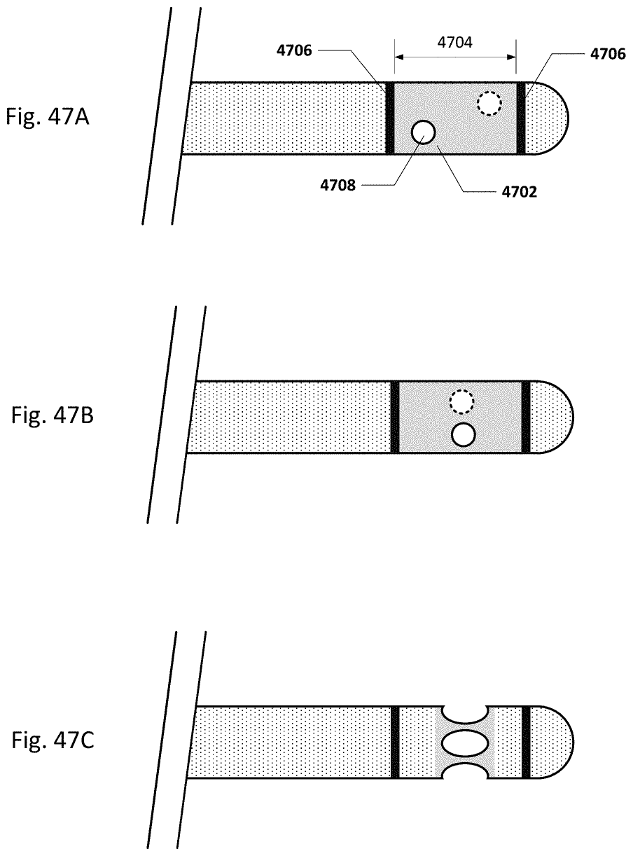

Some embodiments of a diffuse injectate exit port may include multiple openings circumferentially. FIGS. 47A-C show embodiments of the vascular catheter navigation device which includes diffuse exit port area 4702, sensor(s) 4706, which may be electrode pairs or other sensors, diffuse exit port area length 4704, and openings 4708. Note that openings outlined with a dotted line are on the back side of the device. FIG. 47A, for example, shows 2 openings circumferentially within exit port area length 4704. FIG. 47B also shows 2 openings circumferentially within exit port area length 4704, however in FIG. 47B, the openings are aligned circumferentially, where in FIG. 47A, the openings are staggered circumferentially.

As an example, the exit port may have two or more openings within about a 0.5 cm length. As another example, the exit port may have two or more openings aligned circumferentially within the exit port. As an example, the exit port may have three or more openings within about a 0.5 cm length. As another example, the exit port may have three or more openings aligned circumferentially within the exit port.

FIG. 47C shows an embodiment where the exit port openings cover more than 40% of the circumference of the diffuse exit port. In other embodiments, exit port openings cover more than 30% of the circumference of the diffuse exit port. In other embodiments, exit port openings cover more than 50% of the circumference of the diffuse exit port. In other embodiments, the exit port openings cover more than 30% of the area of the diffuse exit port. In other embodiments, the exit port openings cover more than 40% of the area of the diffuse exit port. In other embodiments, the exit port openings cover more than 50% of the area of the diffuse exit port. In other embodiments, the exit port openings cover more than 60% of the area of the diffuse exit port. The percentage of open area of a diffuse exit port may range from 10% to 99%, although more optimally 30% to 80% by area.

In some embodiments, the length 4704 of the diffuse exit port is greater than about 0.10 cm. In some embodiments, the length 4704 of the diffuse exit port is greater than about 0.25 cm. In some embodiments, the length 4704 of the diffuse exit port is greater than about 0.5 cm. In some embodiments, the length 4704 of the diffuse exit port is greater than about 0.75 cm. In some embodiments, the length 4704 of the diffuse exit port is greater than about 1.0 cm.

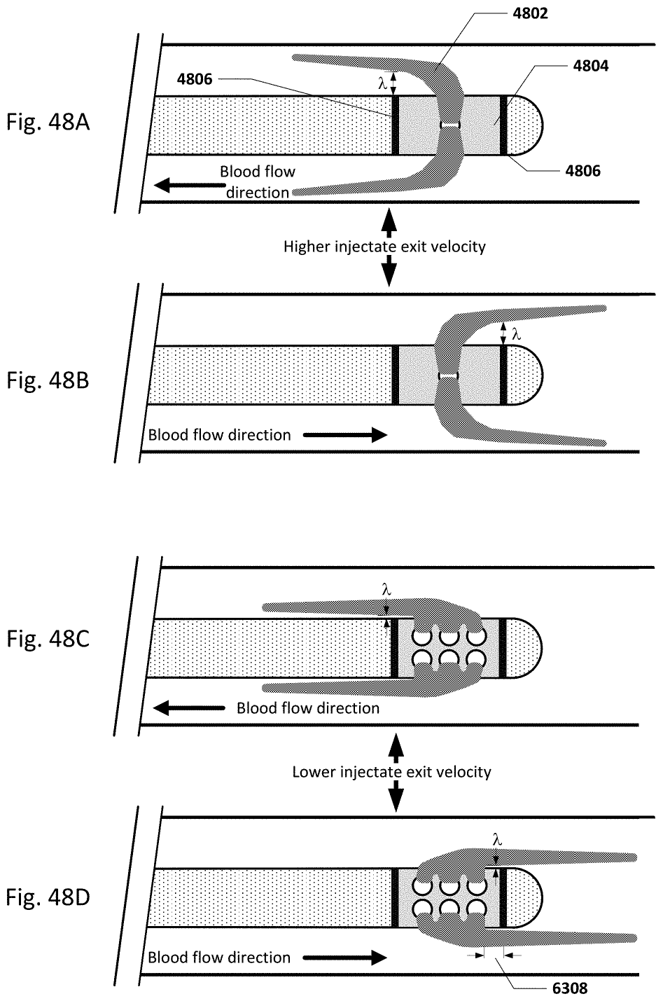

FIGS. 48A-D show that the injectate exit velocity impacts the sensor's ability to sense the injectate parameter within the vessel blood flow. FIGS. 48A and 48B show a 2 dimensional representation of the flow characteristics of injectate fluid 4802 as it exits injectate exit port area 4804 at a higher exit velocity. FIGS. 48A and B show the flow of the injectate fluid, at a given infusion flow rate, in opposing blood flow scenarios. The distance, λ, is the approximate distance between the exiting injectate fluid and sensor 4806, within the plane of the sensor. This distance may also be referred to as the boundary distance. FIGS. 48C and 48D show the fluid flow of the injectate, at the same infusion flow rate, exiting the device in a lower injectate exit velocity, for example via a diffuse exit port. Note that the boundary distance, λ, is much smaller in the low injectate exit velocity scenarios. To optimize sensor sensitivity, it is desirable to design the exit port so that X is at or near zero in laminar or less turbulent blood flow situations, and may be greater than zero in turbulent blood flow situations. Utilizing a diffuse exit port design as shown in FIGS. 48C and 48D allows one to control the boundary distance, λ, for any given proximal injectate injection rate, as well as bringing it close to zero in laminar or low turbulence blood flow situations. By choosing the appropriate exit port configuration for a given injectate injection rate, the sensor can perform under a range of blood velocities. The sensors, along with the analytical capabilities of the controller, can detect inline and opposing flow and can detect the difference between laminar and turbulent blood flow conditions.

For example, at an injectate infusion rate of 3 ml/min, the injectate inlet velocity may be around 6 cm/sec and the injectate exit velocity exiting the exit port may be around 1.5 cm/sec. This represents a ratio of inlet velocity:outlet velocity ratio of around 4. Alternatively, at an injection infusion rate of 5 ml/min, the injectate inlet velocity may be around 10 cm/sec and the injectate exit velocity exiting the exit port may be around 2.5 cm/sec. This represents a ratio of inlet velocity:outlet velocity ratio of around 4. Alternatively, the outlet velocity may be in the range of around 0 to 1 cm/sec. Alternatively, the outlet velocity may be in the range of around 1 to 3 cm/sec. Alternatively, the outlet velocity may be in the range of around 1 to 4 cm/sec. Alternatively, the outlet velocity may be in the range of around 1 to 6 cm/sec. Alternatively, the outlet velocity may be in the range of around 1 to 8 cm/sec. The inlet velocity:outlet velocity ratio may be around 4. Alternatively, the inlet velocity:outlet velocity ratio may be around 2-5. Alternatively, the inlet velocity:outlet velocity ratio may be around 1-6. Alternatively, the inlet velocity:outlet velocity ratio may be around 5-10. Alternatively, the inlet velocity:outlet velocity ratio may be greater than around 2. Alternatively, the inlet velocity:outlet velocity ratio may be greater than around 5. Alternatively, the inlet velocity:outlet velocity ratio may be greater than around 10.

In embodiments which include a diffuse exit port, the distance between the exit port and the sensor may be measured from the closest opening to the sensor. For example, in FIG. 48D, the distance between the exit port and the distal sensor would be length 4808.

In some embodiments of the vascular catheter navigation device, it is desirable to incorporate a sensor into or on the vascular catheter itself. This feature enables the user to detect whether the catheter has migrated over time, even after the guidewire/stylet has been removed. By including the sensor and infusion ports on the catheter itself, the need for a guidewire or stylet may also not be necessary. In some of these embodiments, the electrodes are designed such that the vascular catheter may be trimmed at the distal end without sacrificing the electrode and/or sensor function. For example, electrodes may be printed on the catheter with 3d printing technology, conductive ink may be used, metal bands may be attached, or flex circuits may be affixed to the catheter. Conductive plastics can also be co-extruded to create separate traces or electrode.

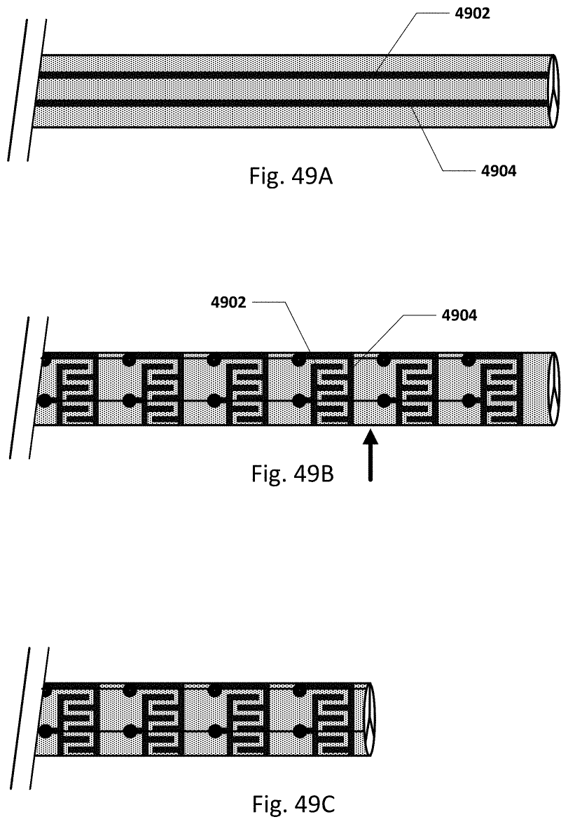

FIGS. 49A-49C show some embodiments of a trimmable vascular catheter with electrodes incorporated into the catheter. FIG. 49A shows electrode 4902 and electrode 4904 that run along the length of a catheter. FIG. 49B shows a vascular catheter with multiple electrode pair areas which can serve as a sensor. FIG. 49C shows the catheter in FIG. 49B after it has been trimmed at the arrow. The distal most sensor or sensors may be used for subsequent injectate parameter sensing during catheter placement. The controller can determine which sensor is the most distal based on the resistance of the sensor loop. The vascular catheter may range from about 42 to about 53 cm in length, and the length trimmed at the distal end may be up to around 6 cm.

Any of the conductivity sensor/electrodes disclosed herein may be incorporated into a rolled printed circuit board which may be wrapped around and attached to the device. Different manufacturing techniques may be used alone or in combination, including plating, masking, lithography, stamping, soldered etc.

The conductivity sensor may also be used to measure the ECG signal of a patient. The two technologies together may be used to identify the device distal tip location within the anatomy. Alternatively, a separate electrode may be used for at least one ECG electrode.

Embodiments with different types of sensors are disclosed herein. It is understood that any type of sensor may be used with any of the embodiments disclosed herein. For example, any of the embodiments disclosed herein may utilize sensors that sense electrical properties, such as conductance or resistance.

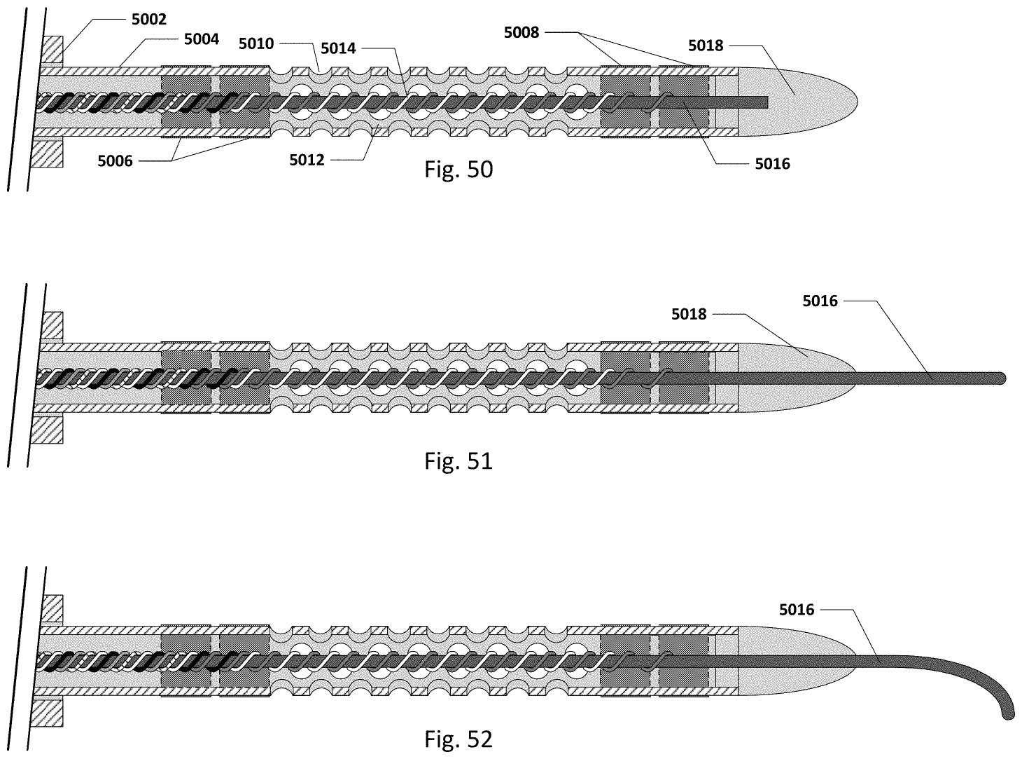

FIG. 50 shows an embodiment of the vascular navigation device which uses electrodes as sensors to sense conductance or impedance of blood and/or mixed blood/injectate. An electrode pair may be considered one sensor. In this embodiment, stylet 5004 is configured to fit within catheter 5002. However, any embodiments shown as a stylet may alternatively be configured into a catheter. Stylet 5004 includes proximal electrode pair, or sensor, 5006 and distal electrode pair, or sensor, 5008. Injectate is injected through a lumen in the catheter or the stylet and exits the stylet via openings 5010 in diffuse exit port area 5012. Electrode leads 5014 in this embodiment are coiled around stiffener 5016 of the stylet. Also shown is stylet end plug 5018. The stylet end plug may have a blunt tip, as shown here, or may incorporate a soft protrusion. Four electrode leads are shown here, but fewer leads may be used. For example, a single lead may connect to both ground electrodes of the two sensors, resulting in three leads instead of four. The electrode leads may be insulated from each other, for example via a coating of each lead.

FIG. 51 shows an embodiment of the vascular navigation device where stiffener 5016 exits beyond end plug 5018. The stiffener may help with guidance of the device through the vasculature. The end plug may be made from adhesive, polymer, metal or other suitable material.

FIG. 52 shows an embodiment of the vascular navigation device where stiffener 5016 ends in a curved portion. The curved portion may be stiff, or more flexible than the more proximal section of the stiffener. The curved portion may be passive, i.e. a set curve in the device, or the curved portion may be active, where the user can change the shape of the curve or the amount of curvature in the tip of the device to help navigate the vasculature. The changing of the curve may be done from the proximal end during the procedure, or may be done prior to the procedure. The curve may be smooth or sharp, i.e. a bend. The curve may be any suitable angle, for example, around 120 degrees, or around 100 degrees to around 140 degrees. The stiffener may or may not exit the device. In other words, a portion, or the entire distal tip outside of the device may be flexible.

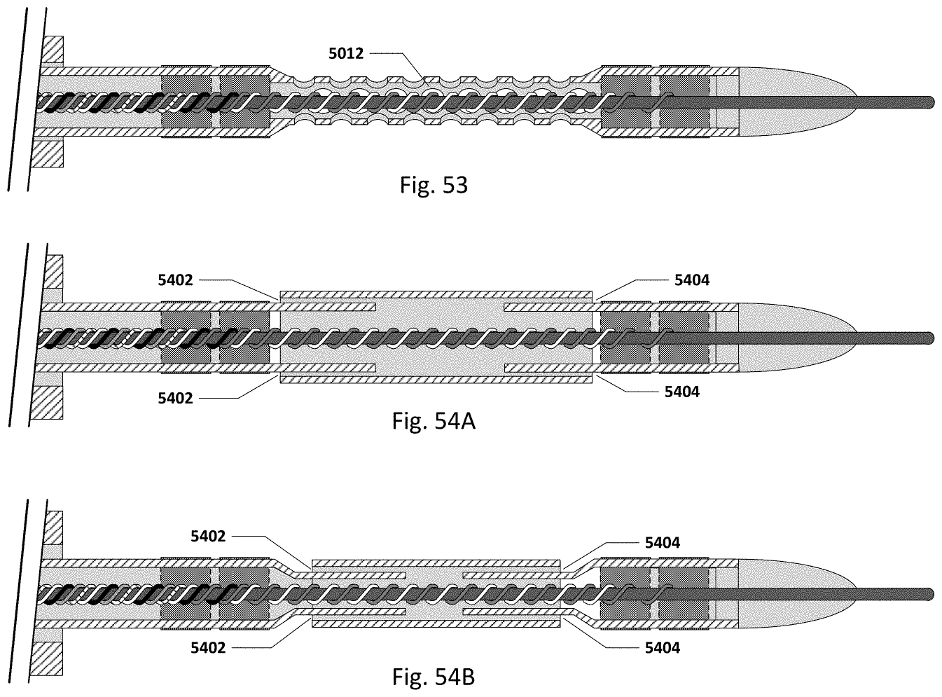

FIG. 53 shows an embodiment of the vascular navigation device with reduced diameter exit port area 5012. For example, the outer diameter of exit port area 5012 may be smaller than the outer diameter of the electrode/sensor area of the device. This embodiment allows for different fluid flow dynamics of the injectate exiting the exit port via the openings. Alternatively, the outer diameter of the exit port area may be greater than the outer diameter of the electrode/sensor area of the device.

FIG. 54A shows an embodiment of the vascular navigation device with a sleeve style exit port area. In this embodiment, the injectate exits the stylet via proximal opening(s) 5402 and/or distal opening(s) 5404. The openings may be annular or comprise one or more openings around the circumference of the device. This embodiment allows for different fluid flow dynamics of the injectate exiting the exit port via the openings. Other embodiments may include baffle(s) or skirt(s) adjacent to the openings to direct the injectate flow as it exits the exit port.

FIG. 54B shows an embodiment of the vascular navigation device with a reduced diameter sleeve style exit port area. In this embodiment, the exit ports are recessed. In other words, the fluid exiting the exit ports exits within a recess of the outer surface of the device. Similar to the embodiments shown in FIGS. 53 and 54A, this may allow the injectate fluid to exit the device in situations where the device is up against a wall of a vessel, or in other confining situations. Embodiments with recessed exit ports may also aid in reducing the boundary distance, λ, as shown in FIGS. 48A-48D. Embodiments with diffuse exit ports, sleeve exit ports and recessed exit ports may reduce the boundary distance, λ.

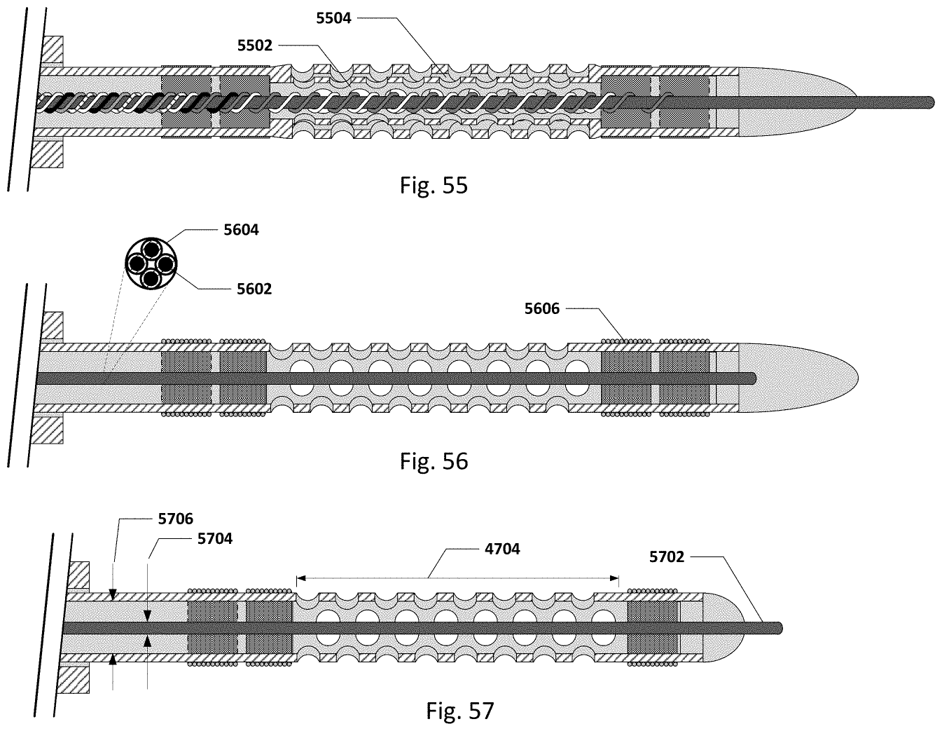

FIG. 55 shows an embodiment of the vascular navigation device with a double layer exit port area. Inner exit port area 5502 in this embodiment is coaxial with, and inside, outer exit port area 5504. This embodiment allows for different flow dynamics of the injectate exiting the exit port area.

FIG. 56 shows an embodiment of the vascular navigation device where the core, or stiffener, includes the leads for the sensors/electrodes. Shown here are 4 insulated leads 5602 within outer sheath 5604. An additional stiffening core may or may not be included in the bundle, which makes up the stiffener. In some embodiments, the insulated leads are stripped and coiled at their distal ends to create coiled electrodes 5606 on the outside of stylet.

FIG. 57 shows an embodiment of the vascular navigation device in which the stiffener is exposed at the distal end forming distal most electrode 5702. This distal most electrode may be incorporated into a coiled atraumatic and/or curved tip.

In these, and other embodiments, the space between OD 5704 of the stiffening core wire and ID 5706 of the outer tube of the device may be important. This defines, at least along a portion of the device, the injection area for fluid injection. The ratio of tube ID to stiffener OD may be around 0.4. Alternatively, the ratio of tube ID to stiffener OD may be around 0.3-0.5. Alternatively, the ratio of tube ID to stiffener OD may be around 0.2-0.6. Alternatively, the ratio of tube ID to stiffener OD may be around 0.1-0.7. In some embodiments, the OD of the stiffening core wire is zero or essentially zero which would cause this ratio to be or approach infinity.

In these, and other embodiments, the ratio of exit port openings total area (for example, port length 4704*% port open area*tube ID 5706*π) to the cross sectional area defining the space between the stiffener OD and the tube ID (π (tube ID 5706/2)²−π (stiffener OD 5704/2)²) may be important. This ratio may be around 4.5. Alternatively, this ratio may be around 1.8-14. Alternatively, this ratio may be around 1.4-20. Alternatively, this ratio may be around 1.2-30. In embodiments where the stiffener OD is zero, this ratio may be around 4. Alternatively, in embodiments where the stiffener OD is zero, this ratio may be around 1.6-13. Alternatively, in embodiments where the stiffener OD is zero, this ratio may be around 1.2-30.

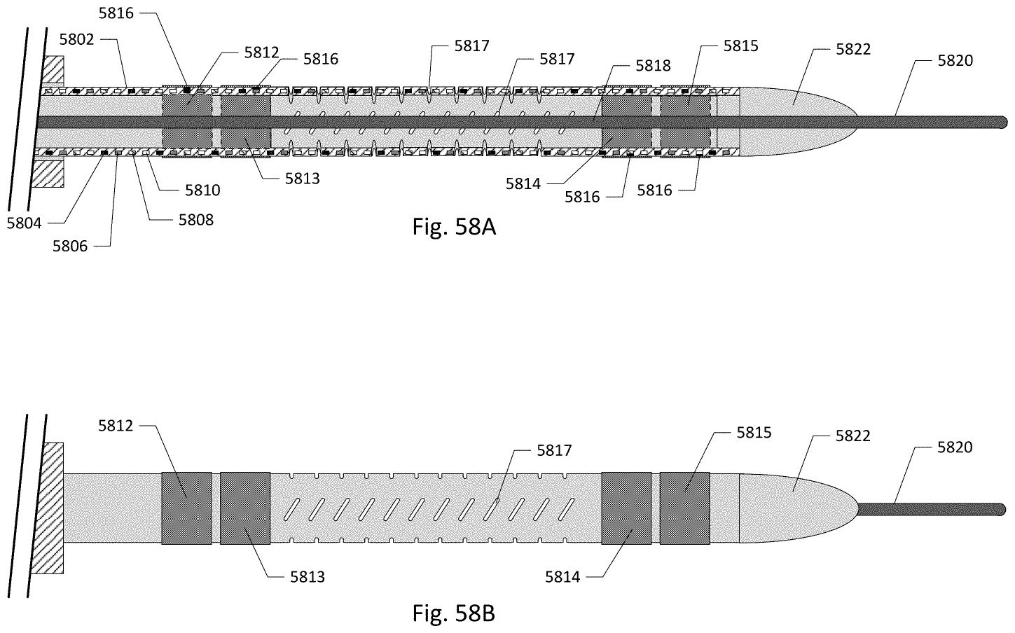

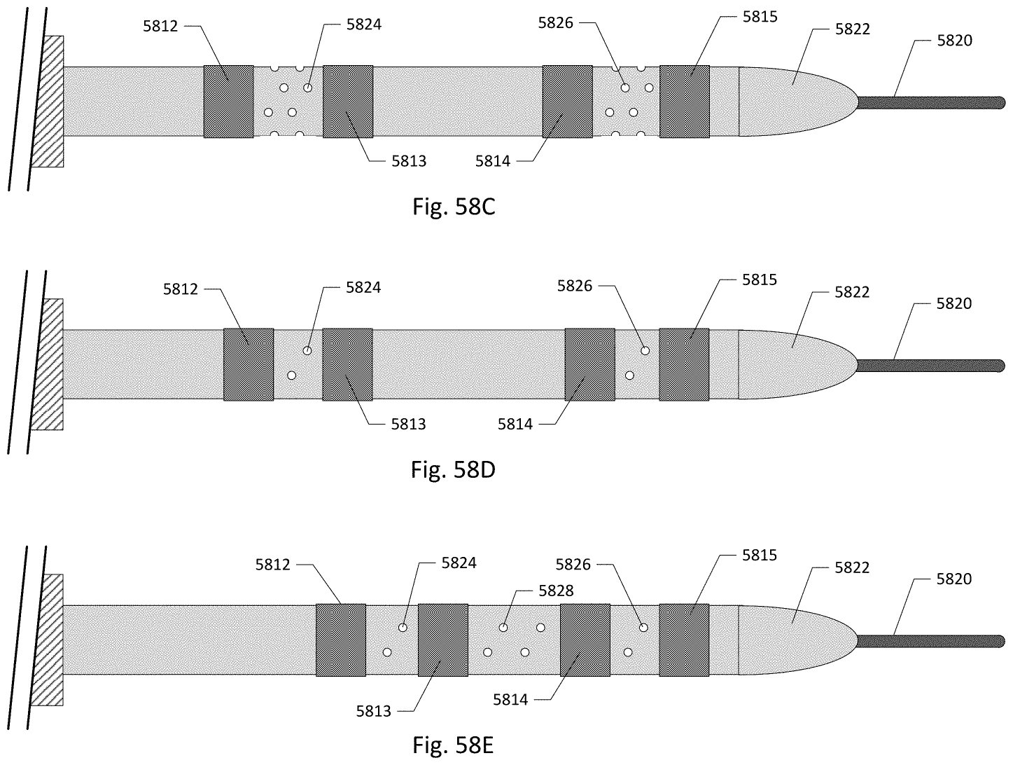

FIG. 58A shows an embodiment of the vascular navigation device in which the electrode leads are separate coils embedded in the outer tubing of the stylet. Coils 5804, 5806, 5808 and 5810 are separate coils embedded in outer tubing 5802. The outer tubing may be a strong thin polymer, such as polyimide, polyethylene, polyurethane or other polymer or material. Coils 5804, 5806, 5808 and 5810 do not contact each other and are separated from each other, or insulated from each other, for example by the material of the outer tubing. They may alternatively or additionally be coated with an insulating material. Coils 5804, 5806, 5808 and 5810 serve as leads for electrodes 5812, 5813, 5814 and 5815. The electrodes are connected to the leads via connection points 5816. The connection points provide an electrical connection between the electrode leads and the electrode bands. Fluid openings 5817 in outer tubing 5802 allow the injected fluid to exit the stylet and enter the blood stream. Fluid openings 5817 are between the coil leads and do not expose the coil leads to fluid. Injected fluid may flow between outer tubing 5802 and stiffener 5818 and out fluid openings 5817. Also shown here is atraumatic tip length 5820, which may be a coil, and end plug 5822. Fluid openings may be round, oblong, or other shapes. The device may include 4 separate coils/leads as shown here, or may have fewer or more coils/leads. In addition, the inner metal stiffener may serve as a lead for an ECG electrode, or an additional coil/lead may be embedded in the wall of the device tubing to serve as a lead for an ECG electrode on the device.

FIG. 58B shows another view of the device in FIG. 58A. This view is a view of the outside of the device, rather than a cutaway view.

FIG. 58C shows another embodiment of the navigation device where the fluid openings are placed between the electrode pair of each sensor, rather than between the sensors. Shown here are proximal electrode pair 5812 and 5813, and distal electrode pair 5814 and 5815. Proximal fluid openings 5824 are between the proximal electrode pair, and distal fluid openings 5826 are between the distal electrode pair. Multiple openings are shown here, but there may be one, two, three, four or more openings. The openings may be round or elongated. The openings may encircle the device 360 degrees, less than 360 degrees, or be along one side of the device. The distal fluid openings may be along a different side of the device than the proximal fluid openings. Fluid openings may also be proximal to the proximal electrode pair and/or distal to the distal electrode pair. There may be one, two, three, four or more openings or sets of openings along the device. These various opening/electrode configurations may exist in a stylet, as shown here, or in a catheter, or both.

FIG. 58D shows an embodiment similar to that shown in FIG. 58C with fewer fluid openings. In some embodiments there may be 4 fluid openings between each electrode pair. The fluid openings may be circumferential around 360 degrees of the device.

FIG. 58E shows an embodiment where mid fluid openings 5828 are present, in addition to proximal fluid openings 5824 and distal fluid openings 5826.

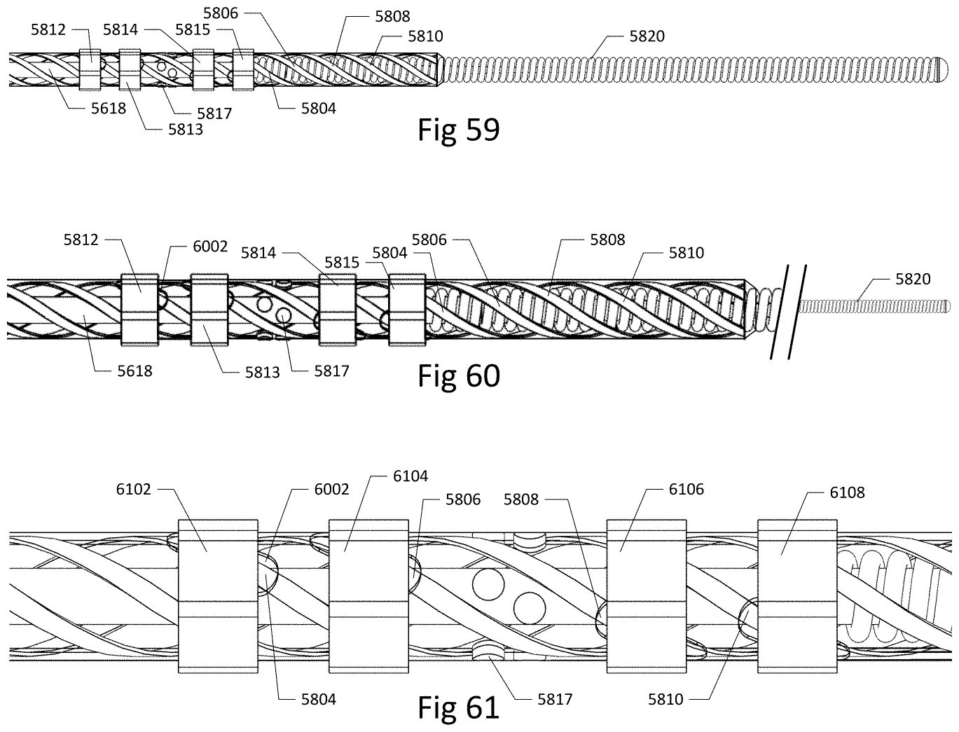

FIG. 59 shows another figure representing an embodiment of the vascular navigation device which is similar to that shown in FIG. 58A. Shown here are coiled electrode leads 5804, 5806, 5808 and 5810, electrodes 5812, 5813, 5814 and 5815, fluid openings 5817, and tip length 5820.

FIG. 60 is a close-up of the embodiment shown in FIG. 59. Also identified here are outer tubing skives or cutouts 6002, which expose electrode leads 5804, 5806, 5808 and 5810, allowing them to make electrical contact with electrode bands 5812, 5813, 5814 and 5815. These cutout openings may be made by skiving the tubing, punching the tubing, cutting the tubing etc. The outer tubing cutouts may extend beyond the electrode band borders, as shown here, or may be completely hidden beneath the electrode bands.

FIG. 61 is a close-up of the embodiment shown if FIGS. 58 and 59. This figure shows more clearly how the individual electrode leads 5804, 5806, 5808 and 5810 connect with individual electrodes 6102, 6104, 6106 and 6108 via cutouts 6002. Also clearly shown are fluid openings 5817 between the coils in the outer tubing, in such a way that the fluid openings do not expose the coil leads within the outer tubing.

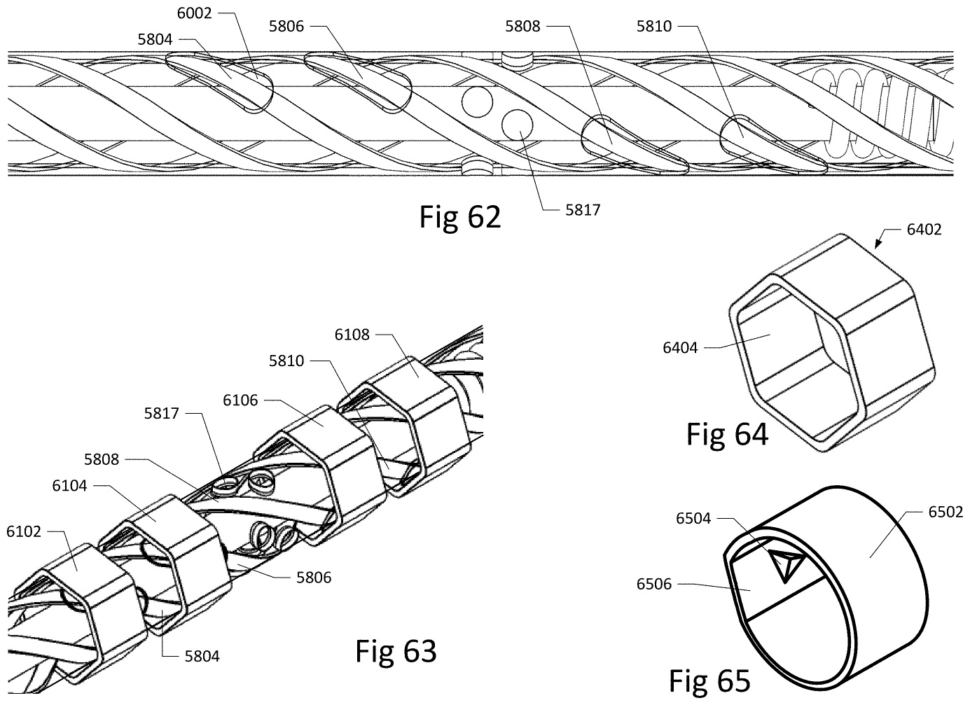

FIG. 62 shows the close-up of FIG. 61 with the electrodes removed. This figure clearly shows electrode leads 5804, 5806, 5808 and 5810 as well as cutouts 6002. Each cutout exposes each separate lead through the outer surface of the outer tubing. The electrode bands can then be connected to the outer tubing and placed in contact with the separate leads, creating 4 separate electrodes on the outside of the stylet.

FIG. 63 shows an angled view of the electrode leads and their respective electrode bands.

Although the distal tip of the device is shown here, the same process of exposing the electrode leads (i.e. skiving) may be used at the proximal end of the navigation device. Electrical connections may also be made at the proximal end of the device via the same leads so that the electrodes are in electrical connection with the controller, via the leads.

FIG. 64 shows electrode band 6402. The electrode bands may have one or more flat areas 6404 which may help increase contact between the electrode lead and the electrode band. Contact may be increased by crimping the band onto the outer tubing of the stylet at the location of the cutout over the electrode lead. Soldering, conductive adhesive or other attachment mechanisms may alternatively be used.

FIG. 65 shows an embodiment of an electrode band which includes a piercing mechanism. Electrode band 6502 includes piercing mechanism 6504 and in some embodiments, flat portion 6506. In some embodiments, a piercing mechanism may be used to avoid the need for cutouts in the outer tubing to expose the electrode lead. Or, a piercing mechanism may be used in conjunction with the cutouts to increase electrical contact. Electrode band 6502 may be placed over the outer tubing of the stylet in the vicinity of the corresponding electrode lead and crimped onto the outer tubing so that the piercing mechanism pierces the outer tubing and contacts the underlying electrode lead, creating an electrical contact between the electrode band and the electrode lead. Piercing mechanisms may include spikes, pyramids, needles or other mechanisms. There may be one or more than one piercing mechanism on an electrode band.

In the embodiments shown in FIGS. 58-63, as well as other embodiments disclosed herein, the distal tip length, for example tip length 5820, may also serve as an electrode. This electrode may be used to collect ECG signals. The entire length of the tip length may serve as an electrode, or the electrode area may be limited to certain portions of the tip length.

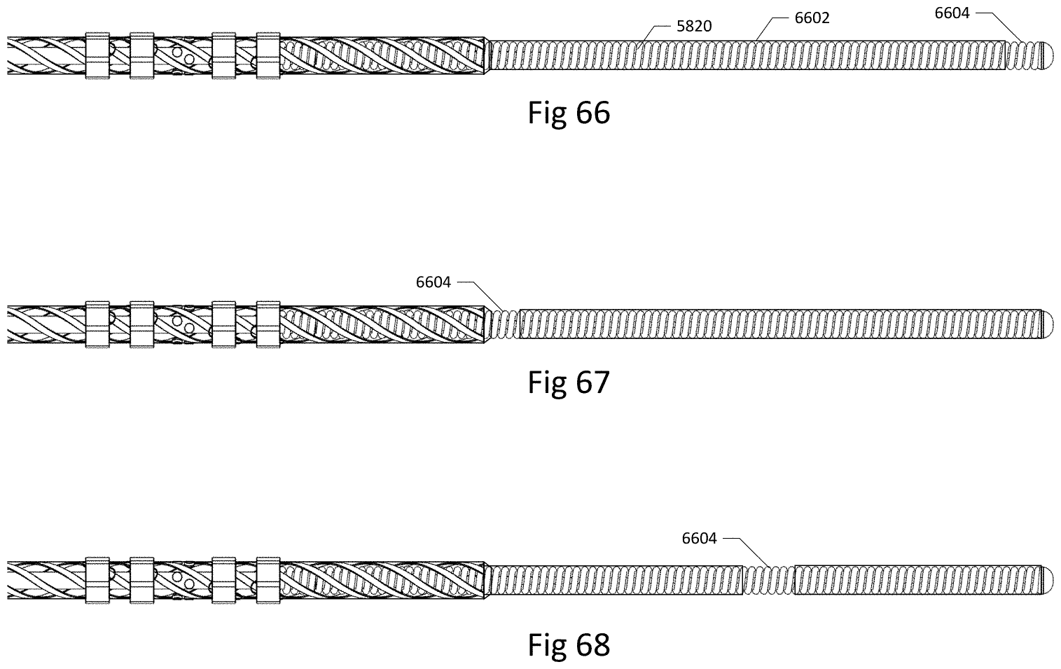

FIGS. 66, 67 and 68 show embodiments of the of the navigation device which include an insulated sheath to limit the electrode area of the distal tip portion of the stylet/catheter. Distal tip length 5820 is shown covered in part by insulating sheath 6602. Insulating sheath 6602 is made out of an electrical insulating material such as a polymer. For example, the sheath may be made out of a heat shrink polymer tubing, such as a polyethylene tubing. Also shown here is exposed area 6604 of distal tip length 5820. The exposed area serves as the electrode since it is not insulated and is therefore exposed to the blood and tissue within the blood vessel. The insulating sheath serves as a means of focusing the electrode so that it is only in a more discreet portion of the tip length. This may allow for a more focused signal from the electrode. In some embodiments a longer length electrode may be desirable, while in others, a shorter length may be desirable.

FIGS. 66, 67 and 68 shown exposed area 6604 at different locations along the distal tip length. In some embodiments, more than one exposed area may be created.

Some embodiments may include a distal tip which includes an electrode, for example for ECG detection, where the ECG electrode only extends part way through the distal tip length. For example, the ECG electrode may include a metal portion, where the distal more length of the distal tip length is made primarily or partially from polymer. Alternatively, both sections may be made from metal, with an insulated portion between them, which limits the length of the electrode portion. In this way, the ECG electrode may be closer to the sensor electrodes on the device, or the ECG electrode may be further from the sensor electrodes on the device. In some embodiments the distal tip electrode may be around 5 mm from the distal most sensor electrode. In some embodiments the distal tip electrode may be around 5-10 mm from the distal most sensor electrode. In some embodiments the distal tip electrode may be around 1-10 mm from the distal most sensor electrode. In some embodiments the distal tip electrode may be around 5-20 mm from the distal most sensor electrode. In some embodiments the distal tip electrode may be around 10-20 mm from the distal most sensor electrode. In some embodiments the distal tip electrode may be around 20-30 mm from the distal most sensor electrode.

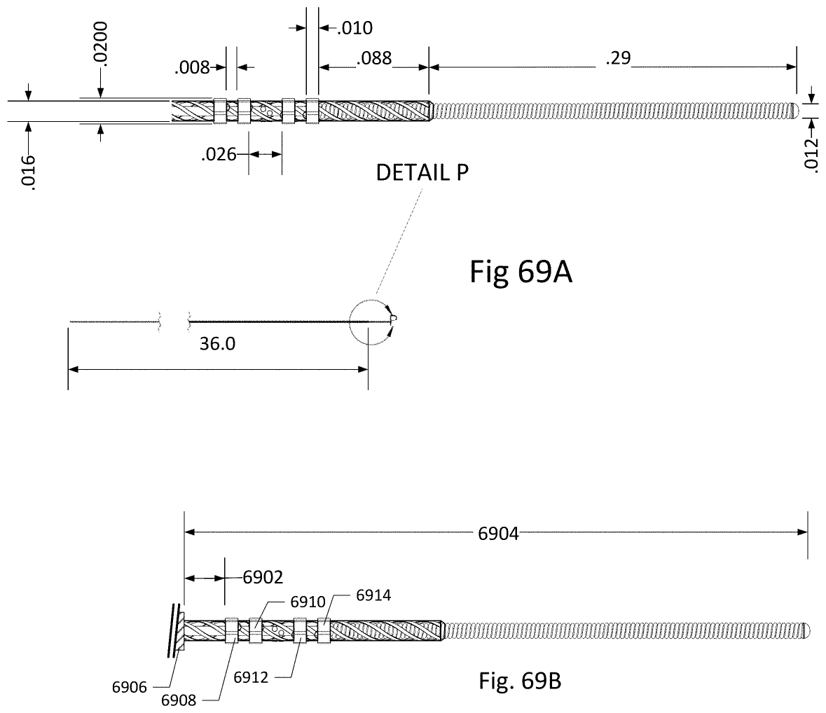

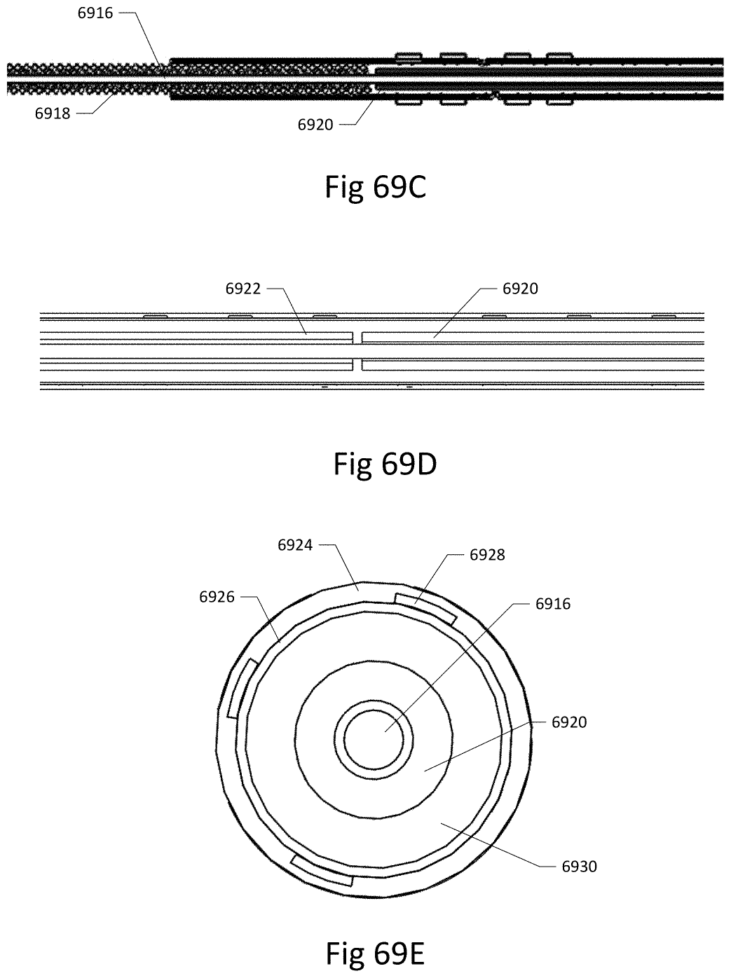

FIG. 69A shows some example dimensions associated with some embodiments of the navigation device. The dimensions shown here are in inches. Note that the dimensions may vary from what is shown here.

In some embodiments, where the sensors are on a guidewire or stylet, the guidewire or stylet extends beyond the distal end of the vascular catheter during placement of the catheter. This allows the sensors on the guidewire/stylet to contact the blood, the infused fluid and the blood/fluid mixture.

FIG. 69B illustrates the relevant distances between the distal tip of the catheter and the stylet/guidewire extending beyond the distal tip of the catheter. Distance 6902 is the distance between the distal tip of the catheter and the proximal edge of the most proximal electrode 6908 of electrode pair 6908 and 6910. Also shown are distal electrode pair 6912 and 6914. Distance 6904 is the distance between the distal tip of the catheter and the distal end of the sensing stylet/guidewire.

In some embodiments, distance 6902 may be around 5 mm. In some embodiments, distance 6902 may be around 0-5 mm. In some embodiments, distance 6902 may be around 5-10 mm. In some embodiments, distance 6902 may be around 10-20 mm. In some embodiments, distance 6902 may be around 20-30 mm.

In some embodiments, distance 6904 may be around 15 mm. In some embodiments, distance 6904 may be around 10-15 mm. In some embodiments, distance 6904 may be around 10-20 mm. In some embodiments, distance 6904 may be around 5-20 mm. In some embodiments, distance 6904 may be around 0-20 mm. In some embodiments, distance 6904 may be around 10-30 mm. In some embodiments, distance 6904 may be around 10-40 mm. In some embodiments, distance 6904 may be around 10-30 mm. In some embodiments, distance 6904 may be around 5-40 mm.

In some embodiments, distance 6912 and/or distance 6914 may be variable, so change at different points during the placement, be different for different patients or for different anatomy, or in response to different alerts, etc. In some embodiments, the distance is known, either by a marker or registration mechanism, for example at the proximal end of the catheter/guidewire/stylet, or by fixing the distance before placement, while the catheter/stylet/guidewire are outside of the body.

The leads may be made of any suitable conductive metal including stainless steel, copper, etc. The coil/leads may be made out of flat ribbon, round wire, or other cross-sectional shapes. The leads may be connected to electrode bands on the outside of the tubing by skiving the tubing to reveal one lead/coil which is to be electrically connected to the electrode band. The connection may be made with conductive adhesive, direct physical contact, solder, etc. The coils/leads may be insulated from each other by a portion of the tubing wall, or by an insulating coating on the coil/lead. This embodiment allows for several electrode leads running at least a portion of the length of the device while conserving space. The leads also serve to reinforce the tubing, allowing it to have more torque, kink resistance and crush resistance.

In some embodiments, an inner guidewire assembly is constructed first using a small diameter wire welded to a flexible coil with an atraumatic ball tip. A hypodermic tube is assembled over the small diameter wire creating stiffness in the proximal length of the stylet. In addition, one or more flexible tubing elements are assembled over the small diameter wire, distal to the hypodermic tubing to create a gradual stiffness transition for kink resistance. The flexible tubing(s) may be affixed to the hypodermic tubing and/or the small diameter wire using adhesive and/or a crimping process. The full guidewire assembly is then assembled inside the coil reinforced polyimide tubing with crimped electrodes.

FIG. 69C shows an embodiment with small diameter wire 6916, flexible coil 6918, ball tip (not shown), hypodermic tube 6920.

FIG. 69D shows flexible tubing element 6922.

FIG. 69E shows outer tubing (which may be polyimide) 6924, inner tubing liner (which may be polyimide) 6926, coil lead 6928 in wall of outer tubing, small diameter wire 6916, hypodermic tube 6920 or flexible tubing 6922 (depending on point along length of device) and infusion pathway 6930.

Some example dimensions and materials:
“Small diameter wire”=NITI #1, 0.003″ OD, 36″ long
“Flexible Coil”=0.012″ OD, 0.0024″ wire DIA, 0.0034″ Pitch, 8 mm long
“hypodermic tube”=304v SS, 0.008″ OD x 0.004″ ID×32″ long
“flexible tubing”=Polyimide, 0.008″ OD x 0.005″ ID×2″ long

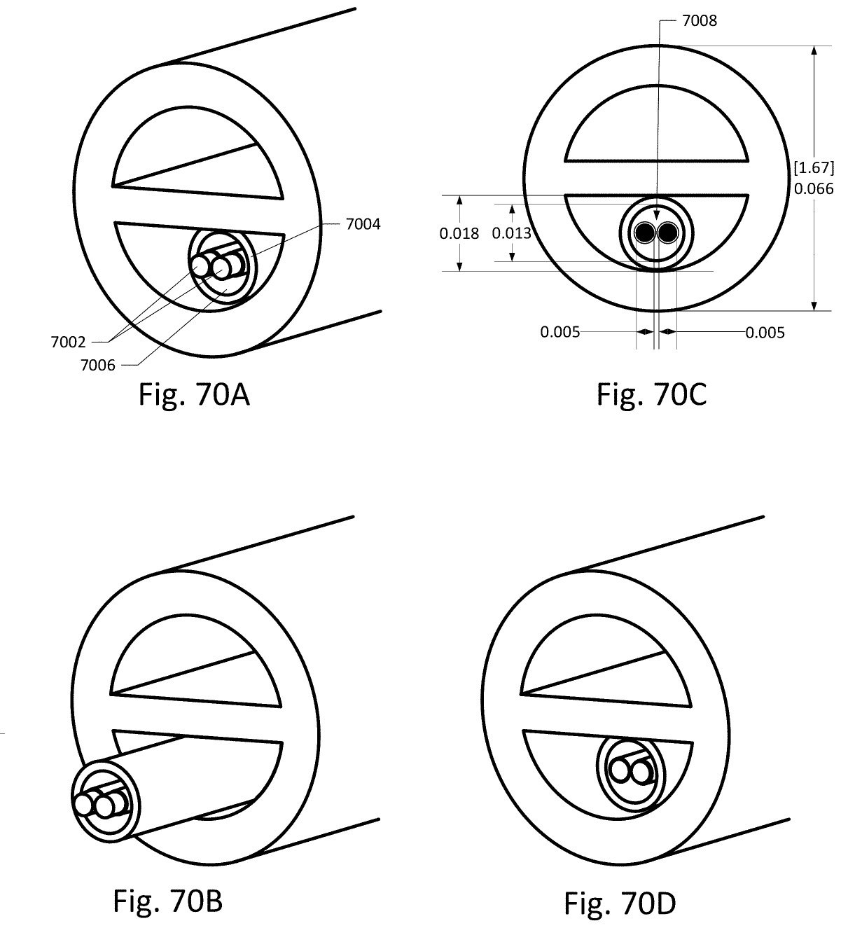

Although several embodiments of the navigation device disclosed herein are shown with a sensing stylet, any of the embodiments disclosed herein may be in the form of a sensor catheter or a sensing catheter/stylet combination. FIGS. 70A-73D show several embodiments of the navigation device, some of which include a sensing catheter.

FIG. 70A shows an embodiment of the navigation device where two electrodes 7002 are comprised of two wires which may be made of any electrically conductive material including, but not limited to: stainless steel, nitinol, gold, copper, copper plated gold, electrically conductive thermoplastic, electrically conductive thermoset, or other conductive materials. The wires are shown here with an insulated coating, but may or may not have an insulating coating. This coating thickness may control the gap of the electrodes which relates to the sensitivity of the sensor. This gap can be anywhere from 0.0001″ to 0.010″, or alternatively around 0.0005″ to 0.0025″. The two electrodes may be made of different materials and may be different sizes. For example, one electrode may be 0.007″ stainless steel, to help with rigidity, and the second electrode may be 0.005″ gold or gold plated copper wire to help with electrical properties. Shown here also is stylet housing 7004 and fluid port 7006 (the space in one lumen of the catheter between the stylet and the interior of the lumen).

The two electrodes may be placed within an elongate body, such as a stylet, guidewire and/or catheter. This elongate body may be fabricated of any suitable material including: polyurethane, polyimide, reinforced polyimide, Polyether ether ketone (PEEK), or other thermoplastics or polymers. An injectate, or infusate, such as water, saline, D5 W or other fluid, may be infused between the electrodes and the inner surface of a lumen of the elongate body. An electrical signal is relayed to a controller which is in communication with the electrodes and may be located remotely. The signal is used by the controller to determine the location of the system within the body based on the response of the sensor to the injected infusate, within a blood vessel (or other cavity) within the flow of blood. The blood is diluted by the infusate and the signal detected by the electrodes depends on the flow characteristics of the blood.

FIG. 70A shows the electrodes with insulation, which is stripped at the distal ends to expose the electrodes. In this Fig, the stylet tip is flush with the tip of the catheter. The catheter shown is a 5 French dual lumen catheter, however any catheter size and any number of lumens may be used.

FIG. 70B shows the stylet of FIG. 70A extended very slightly (0.005″) beyond the distal end of the catheter. The sensor may function with the stylet extended beyond the distal end of the catheter, or flush with the distal end of the catheter, as well as retracted inside the distal catheter tip.

The infusion lumen may be created by the space between the electrodes and the inner wall of a lumen of either the stylet or alternatively, the inner wall of a lumen of the catheter.

FIG. 70C shows some example dimensions of one embodiment of the navigation system. Shown here: Outer diameter of the stylet: 0.018″ (Range 0.005″ to 0.040″), inner diameter of the stylet: 0.013″ (0.004″−0.038″), outer diameter of the electrodes: 0.005″ (0.001″−0.020″), and the insulation thickness on the electrodes: 0.0003″ {0.0001″ to 0.003″), which creates controlled minimum gap 7008 of double the insulation thickness.

FIG. 70D shows an embodiment where the electrodes are not stripped at the end but rather cut flush with the insulated coating. The insulation on the wire and the wire end itself are shown flush with the stylet casing/housing as well as the distal tip of the catheter. Alternatively, the stylet housing/electrodes/catheter tips may not all be flush.

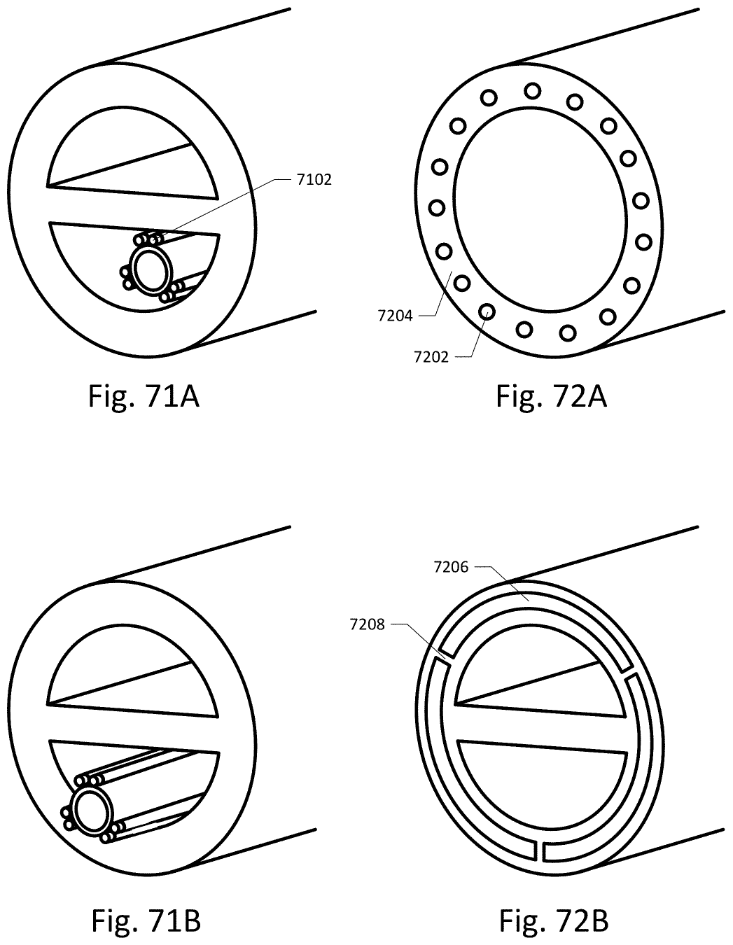

FIG. 71A illustrates an embodiment of the navigation device with three wire pairs each placed approximately 120 degrees radially from each other around the stylet. Each pair of wires acts as a sensor. The wire diameters may range from 0.0005″ to 0.005″ and the spacing between the wires may range from 0.0003″ to 0.003″. The signal from the sensors/electrodes is relayed to the controller which determines the location of the system in the body based on the response of the sensor to the infusate injection and the dilution and/or flow characteristics of the infusate in blood.

These embodiments may include one pair of electrodes, two, three (as shown in FIG. 71A), four or more than 4 pairs of electrodes. The multiple sensors/electrodes may aid in determining the changes in blood flow conditions independently of the orientation of the catheter or stylet. The electrodes and/or electrode leads may be joined with the elongate body (stylet casing or catheter wall) in an extrusion process, a dipping process, a braiding process, a co-molding process or any additional processes. Alternatively the electrodes may be introduced separately, after the extrusion of the stylet casing or catheter wall.

FIG. 71A illustrates an embodiment in which the electrodes are flush with the distal end of the catheter. FIG. 71B illustrates an embodiment in which the electrodes, and stylet, are slightly extended beyond the distal end of the catheter. The electrodes may alternatively be slightly receded within the distal end of the catheter.

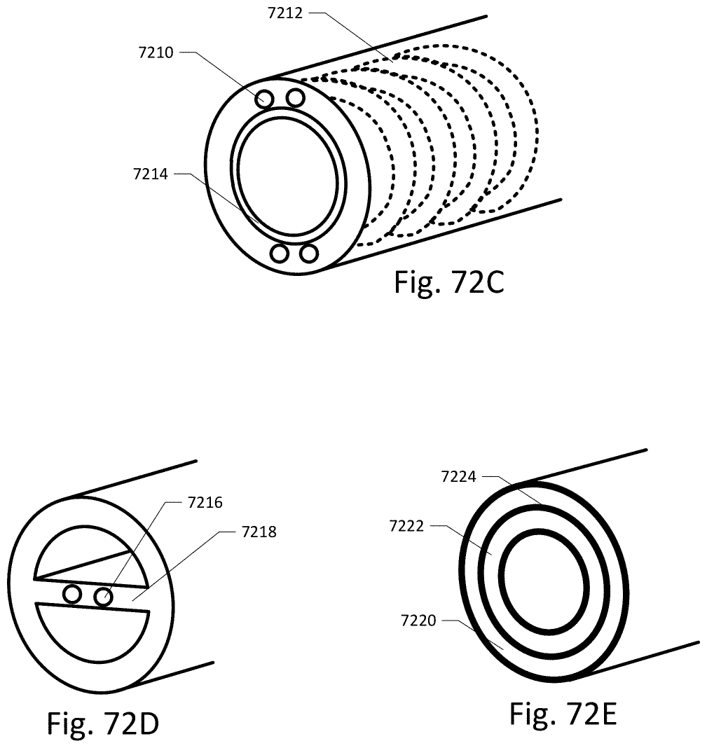

FIG. 72A shows an embodiment with the electrodes or electrode leads embedded into the wall of a catheter itself. In this embodiment the catheter may be extruded polyurethane, or other suitable material, including other polymers. In some embodiments, the catheter tubing is not extruded, such as with polyimide tubing. The electrodes/leads may be copper, gold plated copper, gold, stainless steel, conductive thermoplastic, conductive thermosets, or other suitable conductive materials. The electrodes/leads may be joined with the elongate body in an extrusion process, a dipping process, a braiding process, a coiling process a co-molding process or any additional processes. The electrode/leads may be coiled, braided, parallel or configured in any suitable way. The electrodes/leads are insulated from each other, so they do not contact each other. Any number of electrode/electrode leads may be present.

In embodiments where the electrodes (sensors) are incorporated into the catheter vs. the stylet, the sensors (as part of the catheter) may remain in place in the body for a longer period of time, for example days or weeks, or for as long as the vascular catheter is required for the patient. In these embodiments, the system may detect position in the body during insertion as well as the location of the catheter in the body after the catheter has been inserted and is dwelling in the vascular for the long term. This is useful to detect migration of the catheter from its desired location. The sensors may also be able to detect an increase in thrombus formation on the tip or elsewhere on the catheter, and as a result, indicate when it is time to replace the catheter.

FIG. 72B shows an embodiment of the navigation device with multiple electrodes/leads embedded into the elongate body (catheter or stylet) wall. The electrical conductivity between any electrode pair can act as an individual sensor. These multiple sensors may add redundancy to the system and may help determine blood flow conditions in multiple catheter orientations. For example, where the catheter/stylet is adjacent to a vessel wall, situated at 45 degrees with respect to the axis of a vessel, in very small vessels, etc. The electrodes/leads are insulated from each other, so they do not contact each other.

FIG. 72C shows an embodiment where the electrodes/leads are formed via one or more spiral coils in the wall of the catheter or stylet. Shown here are electrodes 7210, leads 7212 as well as optional inner insulation layer 7214. The leads may be fully embedded within the tubing of the device, or layered between an outer and an inner tubing (which may be the insulation layer).

FIG. 72D shows electrodes/leads embedded in the central divider of a dual lumen catheter. Shown here are electrodes 7216 in central divider 7218. Alternatively, the divider may be any divider, rather than a central divider.

FIG. 72E shows two concentric circular electrodes that are insulated from one another, and may run the length of the device (catheter or stylet). The layers shown are conducting electrode 7220, insulator 7224 and second conducting electrode 7222.

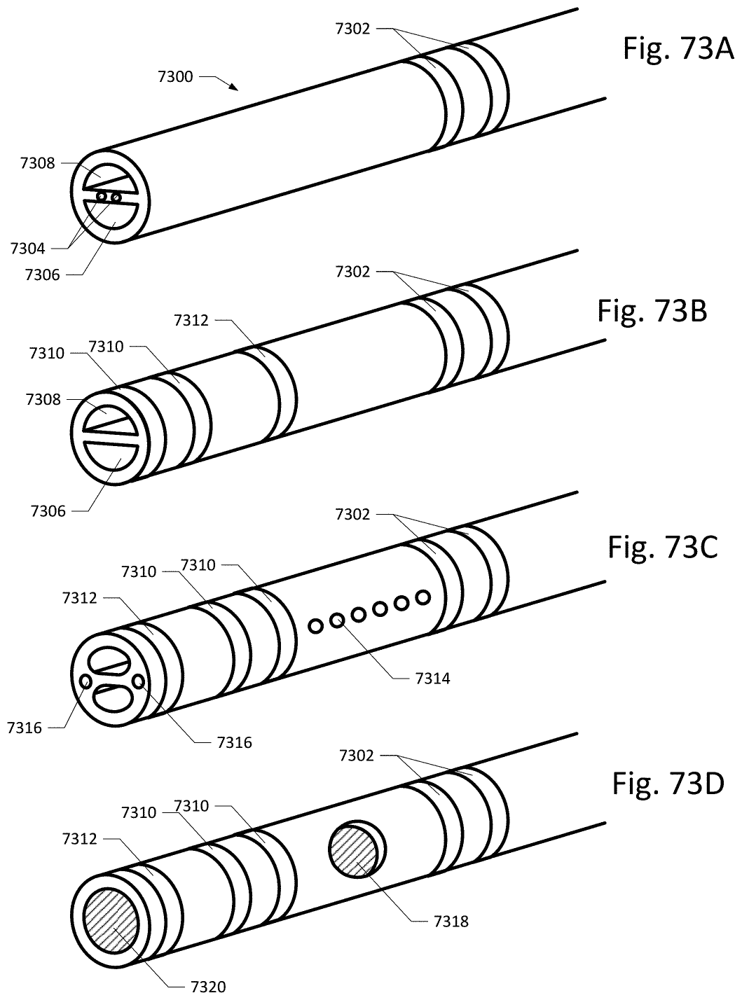

FIGS. 73A-73D show various embodiments of the navigation system which are incorporated into a catheter. In these embodiments, the catheter may remain indwelling in the vasculature after placement. The sensors on the catheter may aid in detecting migration of the catheter so that the correct placement may be reestablished.

FIG. 73A shows catheter 7300 with proximal electrode pair 7302 and distal electrode pair 7304. Lumens 7306 and 7308 may be used for injectate fluid emission and/or regular catheter infusion functions. Distal electrode pair 7304 may be flush with the catheter tip, or may protrude slightly or may be slightly recessed within the catheter tip. 2 lumens are shown, but one lumen, or more than 2 lumens may be present. In this embodiment, the injectate fluid is infused through the catheter and the proximal and distal electrode pairs (sensors) detect signals based on the location of the catheter and the flow of the fluid (a mixture of the injectate fluid and blood) around the sensors.

FIG. 73B shows a sensing catheter embodiment with proximal electrode pair 7302 and distal electrode pair 7310. The distal electrode pair may be flush with, at, or near the distal end of the catheter. Also shown here is ECG electrode 7312 which may be used to detect ECG signals. A second ECG electrode may be placed on the skin of the patient and/or one or more electrodes from electrode pairs 7302 and 7310 may be used also as a second or third ECG electrode. Lumens 7306 and 7308 may be used for injectate fluid emission and/or regular catheter infusion functions.

FIG. 73C shows a sensing catheter embodiment with proximal electrode pair 7302 and distal electrode pair 7310. Also shown is ECG electrode 7312. This embodiment includes injectate fluid openings 7314 between the proximal and distal electrode pairs. This embodiment allows the fluid injectate to exit the catheter between electrode pairs. Shown here also are injectate lumens 7316. Two lumens are shown here, but one lumen, or more than 2 lumens may be used. The lumens may be closed or capped at the distal end of the catheter so the injectate fluid only escapes the catheter between the two electrode pairs. In some embodiments, the 2 injectate lumens are not capped and some injectate also escapes through the distal end of the catheter.

FIG. 73D shows a sensing catheter embodiment with proximal electrode pair 7302 and distal electrode pair 7310. Also shown is ECG electrode 7312. This embodiment includes one or more injectate fluid openings 7318. The fluid openings may communicate with catheter lumen 7320. Catheter lumen 7320 may be used for injectate fluid, and may also be used for other regular catheter infusion functions. In this embodiment, the injectate fluid lumen is open at the distal end of the catheter. Some embodiments may allow for occasional closing of the injectate fluid lumen at the distal end of the catheter, for example, with a valve or balloon, during injection/sensing functions.

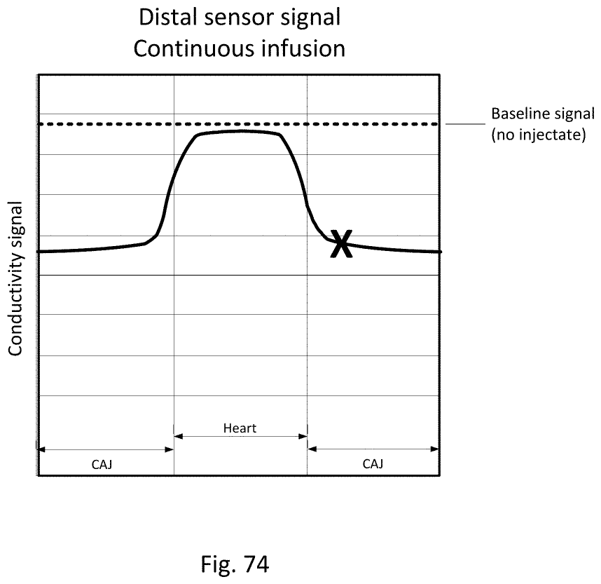

FIG. 74 shows the relationship between the magnitude of the sensor signal and the location of the navigation device in some embodiments. FIG. 74 shows conductivity data collected from the distal sensor of a vascular navigation device which includes conductivity sensors. These data represent a signal from a vascular navigation device during constant infusion of the injectate fluid, as opposed to intermittent or bolus infusion of injectate fluid. In this case, the injectate fluid has a lower conductivity than blood.

This signal has been dampened/filtered to show more clearly the magnitude of the signal as the navigation device is navigated through the vasculature. The distal tip of the device is navigated through the SVC, into the CM, into the heart, and then retracted back through the CAJ and the SVC. The conductivity signal at baseline, with no infusion of injectate medium, is represented by the dotted line. This is generally what the conductivity signal would be if the device were advanced through the vasculature without the injection of any injectate. However, when injectate is continuously infused through the device, so that it exits the device openings near the distal tip, the sensors (in this case electrodes) detect a different conductivity signal depending on the location of the device within the anatomy.

While the device is in the upper part of the SVC, the conductivity is generally lower than baseline, because of the infusion of the injectate which has a lower conductivity than blood. Since there is less turbulence and less mixing in this area of the vasculature, the magnitude of the signal, or the difference between the signal and baseline, is relatively high in this area. The magnitude of this signal may vary with vessel size and/or anatomy. There is also a fairly large magnitude signal as the device enters the CAJ, as is shown here. As the device crosses the superior vena cava/cavo-atrial junction (SVC-CAJ), and enters the right atrium of the heart, the signal magnitude is reduced. In other words, the signal approaches baseline. This is due to the increased flow rate, turbulence and mixing of the blood in the atrium of the heart, which quickly dilutes and eliminates the lower conductivity injectate, so that the sensors do not sense the presence of the injectate. Since the ideal location for the catheter tip is within the CAJ, the user then withdraws the catheter until the magnitude of the conductivity signal again increases, to a point generally represented by the “X” on the curve. In this way, the magnitude of the conductivity signal may be used to locate the navigation device in the CM.

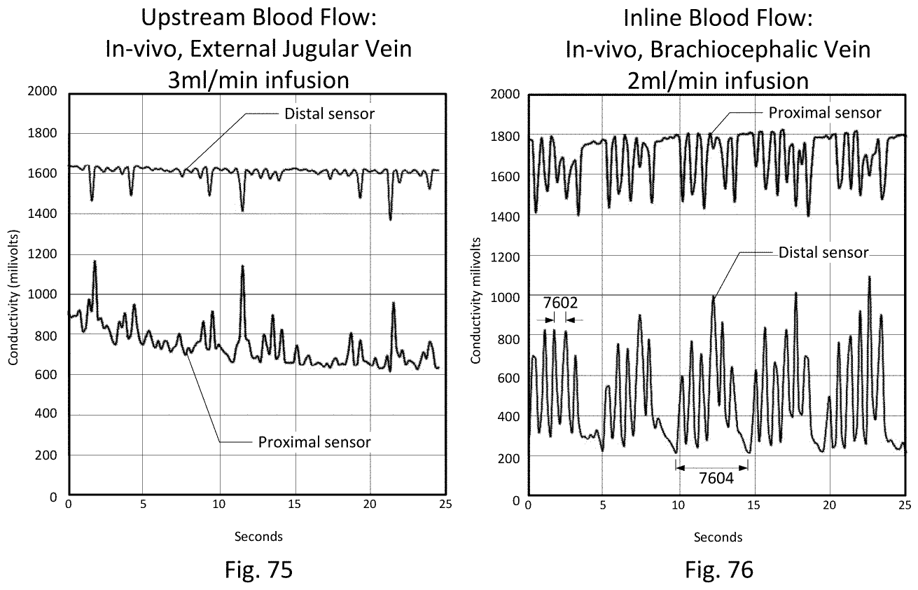

FIGS. 75 and 76 illustrate how the relative magnitude of the signal from the distal and proximal sensors can be used to determine direction of blood flow. FIG. 75 shows the conductivity signal of the vascular navigation device in the external jugular vein of a pig. FIG. 76 shows the conductivity signal of the vascular navigation device in the brachiocephalic vein of a pig. The signals from both the distal sensor and the proximal sensor are shown. Note that blood is flowing with the device, so in the same direction as the advancement of the device, in the brachiocephalic vein, and against the device in the external jugular vein. Blood flowing against the device is an indication that the device is in the wrong vessel and needs to be retracted, so it is important to be able to identify this situation.

In the situation where the device is in the wrong location, where blood is flowing against the device, the signal from the proximal sensor is a larger magnitude from baseline (lower conductivity) than the signal from the distal signal. This is shown in FIG. 75. In the situation where the device is in a blood vessel where blood is flowing with the device, the signal from the proximal sensor is a lower magnitude from baseline (higher conductivity) than the signal from the distal signal. This is shown in FIG. 76. By analyzing the relative magnitude of the distal and proximal sensor, the controller can determine whether the device is in a vessel with blood flowing in the wrong direction (either an incorrect vein or an artery).

FIG. 76 also clearly shows the pulsatile nature of the signal in some locations within the vasculature. Here, in the brachiocephalic vein, the pulsatile nature of both the proximal sensor signal and the distal sensor signal can be seen. Both the respiratory pulse and the heart pulse can be seen in these signals, each having its own frequency. The heart rate frequency has pulse length 7602 and the respiratory frequency has pulse length 7604. Fourier transforms or other mathematical methods can be used by the controller to extract the various frequencies from the sensor signals to determine heart rate and respiratory rate, in addition to the magnitude and relative magnitude of the sensor signals.

A steady injection of the injectate was used to obtain these data in a pig. Steady, varying or intermittent injection of the injectate may be used.

The pulsatility of the sensor signal depends on the location of the device within the vasculature. For example, smaller vessels may produce a more pulsatile signal than very large vessels, or the heart. Therefore, the pulsatility of the sensor signal may also be used to locate the vascular navigation device. Signal pulsatility, signal magnitude and/or relative signal magnitude may be used to locate the device within the anatomy.

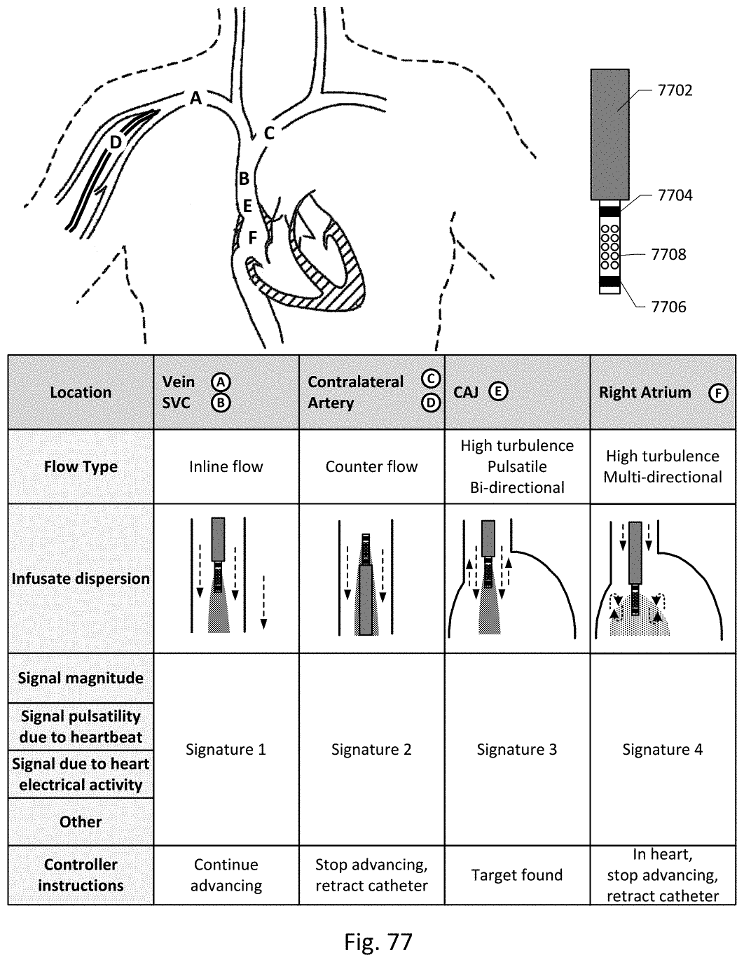

FIG. 77 shows the different types of flow that may be encountered in the vascular system when placing a vascular catheter: inline flow, counter flow, high turbulence bi-directional flow and high turbulence multi-directional flow. FIG. 77 also shows the different types of signals that the vascular navigation device may sense or monitor, including: signal magnitude, signal pulsatility, signal due to electrical activity and other signal types. The controller of the vascular navigation system uses one or more of these signal types to identify where the device is within the anatomy and also communicates to the user instructions based on the sensed location.

Some examples are provided herein, but it is understood that the signal signature may be different than the examples, and may incorporate fewer or more or different signal types. The controller may incorporate the one or more signal types as absolute values, or relative values. The relative values may be relative to another point in time, or relative to another signal type, or relative to the same signal type from a different sensor. For example, the controller of the vascular navigation device may determine that the distal tip of the device is in an artery based on a high proximal sensor signal magnitude. Alternatively or additionally, the determination may be based on an increase in the proximal sensor signal magnitude from a previous time/location of the device. Alternatively or additionally, the determination may be based on the proximal sensor signal magnitude relative to the distal sensor signal magnitude, where the proximal sensor signal magnitude may be higher in an artery. Any of the signal types disclosed herein may be analyzed similarly, either absolutely or relatively or both.

The anatomical diagram shows the location of an entry vein (A), the SVC (B), a vein with contralateral blood flow (C), an artery (D), the CAJ (E), and the right atrium (F). As the device is advanced through the vasculature, ideally, it passes through the entry vein, into the SVC, to the CAJ. The vascular navigating system may detect where the device tip is based on one or more signal signatures. The vascular navigation system may also be able to detect the transition of the location of the device from one anatomical area to another. For example, the vascular navigation system may detect when the device has traveled past the CAJ and has entered the right atrium as it is advanced from the CAJ and instruct the user to retract the catheter/device slightly so that it is again in or near the CAJ. The navigation system may detect when the device has passed back into or near the CAJ and indicates to the user that the device is now in its desired location.

It is also possible that during navigation the catheter/device may enter either an artery or a vein with contralateral blood flow, such as locations (C) or (D). If this occurs, the system may detect, based on one or more signal signatures, that the device is in the wrong location and indicate to the user that he/she should retract the device until the system signals that the device is no longer in the wrong location.

A representation of the vascular navigation device is shown with catheter 7702, proximal sensor 7704, distal sensor 7706 and infusion port area 7708.

The chart in FIG. 77 shows some different types of signals sensed by the sensors and received by the controller of the system at the different locations in the vasculature. These signal types may include:

1. Signal Magnitude

The signal magnitude is the magnitude of the sensor signal relative to baseline. The signal magnitude used by the controller may be the absolute signal magnitude, or may be the relative signal magnitude. A relative signal magnitude may be relative to the signal magnitude at another time/location within the vasculature or relative to the magnitude signal of another sensor or relative to another point in time, for example, before injectate is injected. For example the controller may use the absolute signal, or the increase or decrease of a signal as the device is advanced through the vasculature. The controller may alternatively or additionally use the relative magnitude of the signal between the distal and proximal sensors—in other words, whether one is a higher magnitude signal than the other and by how much.

2. Signal Pulsatility

The signal pulsatility used by the controller may be the absolute signal pulsatility, or may be the relative signal pulsatility. A relative signal pulsatility may be relative to the signal pulsatility at another time/location within the vasculature or relative to the pulsatility signal of another sensor.

3. Signal Due to Heart Electrical Activity

The signal due to heart electrical activity may be sensed by the sensors. Either one or more than one sensor may pick up the heart electrical activity. The relative signal due to heart electrical activity may be relative in time/location, or the relative signal between two sensors. In general, the signal due to heart electrical activity will be stronger nearer to sinoatrial node at the entryway to the heart.

Other signal types may alternatively or also be used. For example, signal phase (for example, the relative phase of the signal between two sensors) may be used. In another example, pressure, such as vascular pressure, may be used.

The following is an example of how the vascular access device may use signal signatures to locate the device within the anatomy. As the device/catheter is being advanced properly through a vein and the SVC, the blood flow is inline, the proximal sensor signal magnitude may be small or negligible, and the distal sensor signal magnitude may be relatively large (although this may vary depending on the size of the vein). In other words, the ratio between the distal and proximal signal magnitudes shifts to a ratio of generally greater than 1. The pulsatility of the sensor signals may also be relatively large. The signal due to heart electrical activity may be relatively low. The controller may determine from one, or a combination of more than one, of these signal types that the device is in a proper vein and indicate to the user to continue advancing the device.

As the device enters an incorrect vessel, such as an artery or a vein with contralateral blood flow, the proximal sensor signal magnitude may become relatively large, while the distal sensor signal magnitude may become relatively small, in other words, the ratio between the distal and proximal signal magnitudes shifts to a ratio of generally less than 1. The pulsatility of the signals may be large if the device is in an artery, or small if the device is in a contralateral flow vein. The signal due to heart electrical activity may remain relatively low. Based on one or more of these signals, the controller of the vascular navigation system may determine that the device is in an incorrect location, and may be able to determine whether the device is in an artery or vein. The controller indicates to the user that the device should not be advanced further and should be retracted until the sensor signals again indicate that the device is in a vein with inline blood flow.

As the user continues to advance the device through the SVC and into the CAJ, the system will instruct the user to continue advancing until the CAJ or the right atrium is detected. In the CAJ, the magnitude of the distal sensor signal may be relatively high, while the magnitude of the proximal sensor signal may vary. The pulsatility of the signals may be relatively high and the signal due to heart electrical activity may be relatively high. The controller may be able to identify that the device is in the CAJ at this point and instruct the user to stop advancing. Alternatively, the user may be instructed to continue advancing the device as it enters the right atrium of the heart.

The blood flow in the right atrium of the heart is highly turbulent and multi-directional, causing much mixing and quick dilution of the injectate. In this area, the magnitudes of both the distal and proximal sensors signals may be reduced, and the pulsatility of the signals may be reduced. The signal due to heart electrical activity may be relatively low. One or more of these signals may be used by the controller to determine that the device has entered the heart. At this point, the controller will signal the user to stop advancing the device, retract the device until the controller detects that the device is again approximately in, or near the CAJ. The vascular navigation system may automatically control the distance that the device/catheter is retracted to ensure proper location of the distal catheter tip in or near the CAJ. In some embodiments, the vascular navigation device extends a known distance beyond the tip of the catheter, and in this case, the device/catheter may not need to be retracted, as the distal tip of the catheter may be in the CAJ when the distal tip of the vascular navigation device is in the heart.

After the navigation device/catheter have been properly placed, the navigation device may be disengaged from, and removed from the lumen of the catheter.

The controller may determine device location within the vasculature based on signal signatures. One or more than one of the signal types may be used to locate the device. The signal type or combination of signal types used in one location may be different than that used in another location in the anatomy. The controller may also or additionally analyze the signals for particular frequencies representing heart rate, respiratory rate or other factors. This information may also be factored into the controller logic used to locate the device or for other purposes.

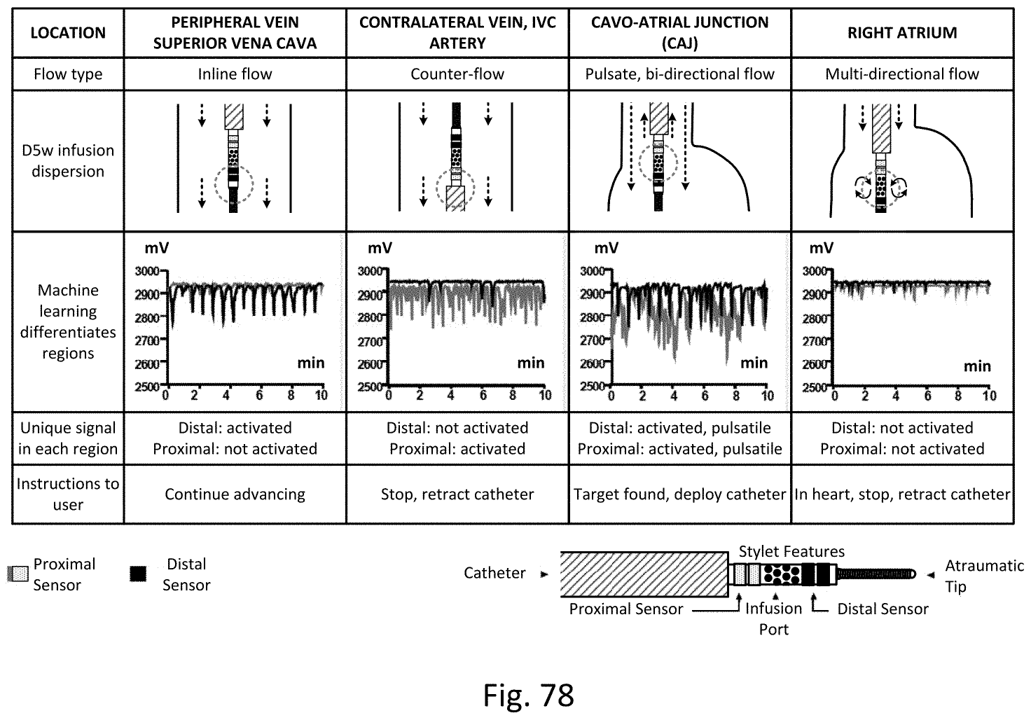

FIG. 78 shows an example of specific signatures received by the controller using some embodiments of the navigation device. The controller uses the data collected to identify the location of the device based on data signatures based on vascular flow. For example, ionic dilution signatures are shown here for of (1) SVC, (2) CAJ, (3) RA, and (4) IVC. Machine learning may be used to analyze the signatures, and to interpret signals based on the analysis.

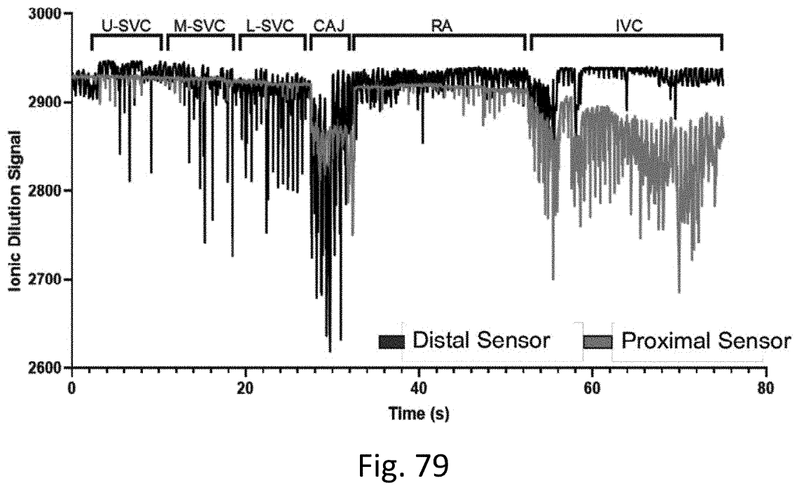

FIG. 79 shows a close up of a graph showing ionic dilution signatures and the transition of the signatures as the device tip enters different areas of the anatomy of a pig. This plot shows the raw time signal from a single animal, which is representative of the larger cohort (n=5). The ionic dilution signal detects the resistance or impedance of the blood and D5 W solution mixture in proximity to each sensor. The periodic nature of each signal is due to the pulsatile blood flow (at the heart rate frequency) interacting with the infused, non-ionic D5 W solution. By using both a proximal and a distal sensor, the vascular access device is able to measure a unique ionic dilution signature at each vascular region.

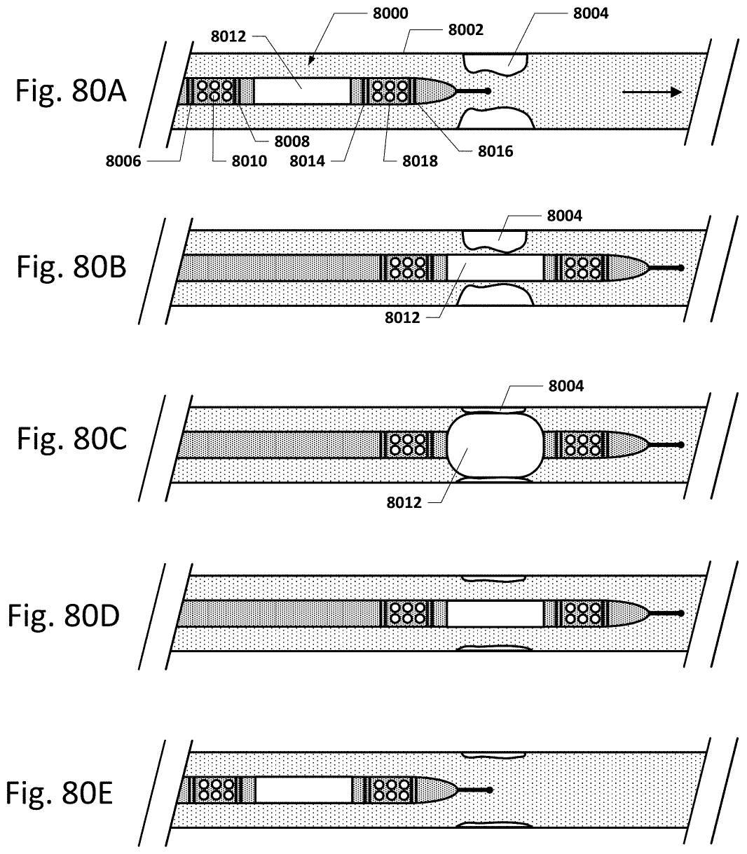

FIGS. 80A-80E show an embodiment of the vascular navigation device which is incorporated into a balloon catheter or stylet, such as an angioplasty catheter or stylet. Shown here is angioplasty device 8000 inside blood vessel 8002. Plaque 8004 is shown which may be impeding blood flow through the vessel. Two sets of sensors are shown, proximal sensor pair 8006 and 8008, as well as distal sensor pair 8014 and 8016. Proximal injectate openings 8010 are shown between the proximal sensor pair, and distal injectate openings 8018 are shown between the distal sensor pair. Angioplasty balloon 8012 is also shown in the deflated state.

The proximal and distal injectate fluid openings may be fed by separate lumens within the device, or the same lumen. By having 2 pairs of sensors and injectate openings, the device is able to sense blood flow characteristics both proximal to, and distal to, the plaque, both before, and after balloon dilatation of the plaque.

FIG. 80A shows the angioplasty device approaching the plaque. Blood flow characteristics may be assessed proximal to the plaque using one of both of the distal and proximal sensor pairs.

FIG. 80B shows the angioplasty device within the plaque area. In this position, blood flow characteristics may be assessed both proximal to, and distal to, the plaque before dilatation.

FIG. 80C shows balloon 8012 inflated to dilate the plaque. In this position, blood flow characteristics may be assessed both proximal to, and distal to, the plaque during dilatation. This information may be useful in assessing whether the plaque is being fully dilated.

FIG. 80D shows the angioplasty device within the plaque area after dilatation. In this position, blood flow characteristics may be assessed both proximal to, and distal to, the plaque after dilatation. This information may be useful in assessing whether the plaque was successfully dilated. For example if the plaque is no longer impeding blood flow, the flow characteristics may be similar for both the proximal and distal sensor pairs.

FIG. 80E shows the angioplasty device as it is being removed from the plaque area. In this position, blood flow characteristics may be assess and compared to the blood flow characteristics before the angioplasty was performed. This will aid in assessing the effectiveness of the angioplasty dilatation.

Since the ionic dilution signal is not affected by arrhythmia, some embodiments of the vascular access device may be used in arrhythmia patients to accurately locate the device.

Embodiments of the vascular navigation device may use a constant infusion rate of the injectate, an intermittent injection, a varying infusion rate, or various infusion rates, depending on anatomy, patient, location within the anatomy etc. For example, a steady injection rate may be used for navigation, until the device detects turbulent flow. The device may then either signal the user to, or automatically, increase or otherwise change the injectate infusion rate. The resulting sensor data may be used to confirm the location of the device in the heart, vs. the thoracic junction or other bifurcation or elsewhere in the anatomy.

In some embodiments, the infusion rate of the injectate may be constantly varied so that more data at different injection rates may be constantly collected and analyzed. For example, the injection rate may vary in a sine wave, a constant increase or a constant decrease or other function.

In some embodiments, the infusion rate of the injectate may be automatically tuned so that the sensor signals are maximized. This “tuned” infusion rate may be determined by patient, by anatomy, by location in anatomy or any combination of these.

In some embodiments, the infusion rate of the injectate may be varied and tracked so that the signal from the sensors is constant. In this way, the infusion rate may be used to determine vessel parameters including diameter, location of the device, etc.

In some embodiments, the signal signature, and/or the signal magnitude may be used to determine vessel diameter, where a larger signal magnitude generally indicates a vessel of a smaller diameter.

In some embodiments, the health of the vessel may be determined based on sensor signals, infusion rate, or both.

In some embodiments, the controller of the vascular navigation system may use sensor data to collect health data on the patient. For example, the system may be able to assess the hydration level of the patient based on the salinity of the patient's blood. The device can also determine respiratory rate, heart rate, blood flow rate based on the sensor data.

Some embodiments of the vascular navigation system may use data collected to assess the health of the patient, for example the presence or absence or status of heart arrhythmias, valve issues, pulmonary hypertension, deep vein thrombosis, bradycardia or heat block, congenital heart disease of various sorts, ventricular arrhythmias, supraventricular tachycardia, atrial fibrillation, atrial flutter, tachycardia, tre-entrant tachycardia, premature atrial contractions (PACs), premature ventricular contractions, junctional arrhythmias, tricuspid regurgitation, tricuspid stenosis, pulmonary regurgitation, pulmonary stenosis, mitral or aortic stenosis, mitral or aortic regurgitation, atrial septal defect, patient ductus arteriosus, systolic heart failure (or HFrEF), diastolic heart failure (HFpEF), right heart failure, cardiogenic shock, distributive shock, hypovolemic shock, obstructive shock, pulmonary embolism, cardiac effusion, cardiac tamponade, perivalvular leak, subclavian stenosis, jugular vein stenosis, pulmonary vascular shunts, hepatorenal syndrome, hypokalemia, hyperkalemia, digitalis toxicity, superior vena cava syndrome, inferior vena cava syndrome, pneumothorax, lung or mediastinal masses, pleural disease or effusion, diaphragmatic paralysis, compartment syndrome, cirrhosis, angioplasty, aortic aneurysm, arterial bypass, cardiac catheterization, cardiac device monitoring, cardiomyopathy, carotid artery stenting, carotid endarterectomy, computed tomography, congestive heart failure (CHF), constrictive pericarditis, coronary artery bypass surgery, dilated cardiomyopathy, echocardiography, heart transplant, hypertrophic cardiomyopathy, implantable cardioverter-defibrillator (ICD), varicose vein treatment, mitral prolapse, pericardial effusion, restrictive cardiomyopathy, stroke, thrombectomy, ventricular assist devices (VAD) etc. machine learning and or neural networks may be used within one patient or using data from more than one patient to correlate signature signals received and analyzed by the controller with particular disease states or risks.

Some embodiments of the vascular navigation system may help identify positioning or malpositioning of devices such as pacemakers, ECMO (Extracorporeal membrane oxygenation) circuits, intraaortic balloon pumps, impellor based heart pumps, IVC filter placement, umbilical vessel catheters, etc.

Some embodiments of the vascular navigation system are designed to automatically calibrate the system. For example, when first inserted, the device may be able to assess the relative salinity of the patient's blood, the relative vasculature size, blood flow rate, blood viscosity of the patient, etc. The system may automatically run through a range of injectate infusion rates to maximize the sensor signal in a given patient. The system may collect sensor data with zero injectate infusion, and at set or varying rates of injectate infusion. The calibration process may be performed at the beginning of the procedure, or at any time during the procedure. The calibration process may also be performed manually, with or without prompts from the controller.

Some embodiments of the vascular navigation system use device vibration data to help determine device location. Some embodiments control for device vibration.

Some embodiments disclosed herein may be used to determine fluid levels, or hydration level, of a patient. Fluid levels are particularly important when a patient has congestive heart challenges. A lower fluid level may result in lower amplitude pulses in the blood flow, where a higher fluid level may result in greater amplitude blood flow pulses. Other flow patterns may be different between a hydrated patient and a less hydrated patient. These flow patterns can be detected using embodiments disclosed herein. Hydration level can be monitored in a patient over time or compared among patients.

Some embodiments of the vascular navigation system use controller logic to identify signal signatures specific to certain conditions, including the condition where the sensor area of the device is up against a wall of a vessel or in a curve of a vessel. In this condition, it is possible that the sensors are not adequately or circumferentially being exposed to the injectate fluid. It is also possible that the sensors may sense the tissue of the wall of the vessel itself instead of the fluid within the vessel. The controller may identify these situations based on changes of any of the sensor signals disclosed herein and may perform one or more of several functions to change the condition, such as: indicating to the user to move the device forward, backward or rotationally, moving the device automatically, moving the device with respect to the catheter, increasing or decreasing the injectate infusion flow rate, changing the injectate infusion flow rate from pulsatile to continuous or from continuous to pulsatile, changing the sampling and/or driving frequency, etc.

Some embodiments of the vascular navigation system measure a direct physical property, such as blood flow rate, blood pressure, blood flow turbulence, blood flow direction etc. one or more of these direct physical properties may be used to guide device placement. In some embodiments, the measurement of a direct physical property may be combines with the measurement of an indirect physical property, or a surrogate physical property, such as ECG. One advantage of measuring a direct physical property is it is unaffected by heart arrhythmias or other electrical signal issues, and may be used as a “ground truth” measurement, either to be used alone, or in conjunction with other measurements.

Some embodiments of the vascular navigation system include sensors to sense other patient parameters, such as chemical sensors (02, glucose, electrolytes, etc.), temperature sensors, viscosity sensors, blood thickness sensors, pressure sensors, ECG, etc. For example, blood clotting time may be able to be determined after the introduction of blood thinning drugs. These sensors may sense these parameters in real time.

Some embodiments of the vascular navigation system may include algorithms that use different types of signals and/or determinations. For example, by measuring vessel diameter and blood flow rates, as well as determining device position, some embodiments may perform real time estimates of how well drugs are mixing in, or infusing into, the blood stream. In some embodiments, the drug(s), may be the injectate fluid. Midline catheter placement, which may be more affordable and easier to place than PICCS and Central catheter lines, but are different than PICC lines in that they are not placed at the CAJ. By determining the mixing rate, or mixing result of two infusion mediums, the vascular navigation system may determine the device location based on this mixing outside of the CAJ.

Some embodiments of the vascular navigation system may include the ability to determine intravascular pressure. This may be accomplished by introducing the injectate at known pressures, and determining when the inject either is flowing or not flowing, By identifying the injectate pressure at which the injectate starts to overcome the blood pressure to flow into the blood vessel, the intravascular pressure may be determined.

In some embodiments, the vascular navigation system may be used in conjunction with pressure measurements, for example, in conjunction with a pressure wire. The readings from the vascular navigation system may inform the calibration of the pressure wire to avoid drift in the pressure measurements due to bubbles or other factors. For example if the voltage readings from the sensors on the device (also referred to as dilution sensors, or ionic dilution sensors) is constant, but the pressure reading is drifting, the controller may determine that the drift is not real but an artifact. Conversely if the pressure reading is stable and ionic dilution reading is drifting, the pressure signal data may be used to calibrate the ionic dilution sensors.

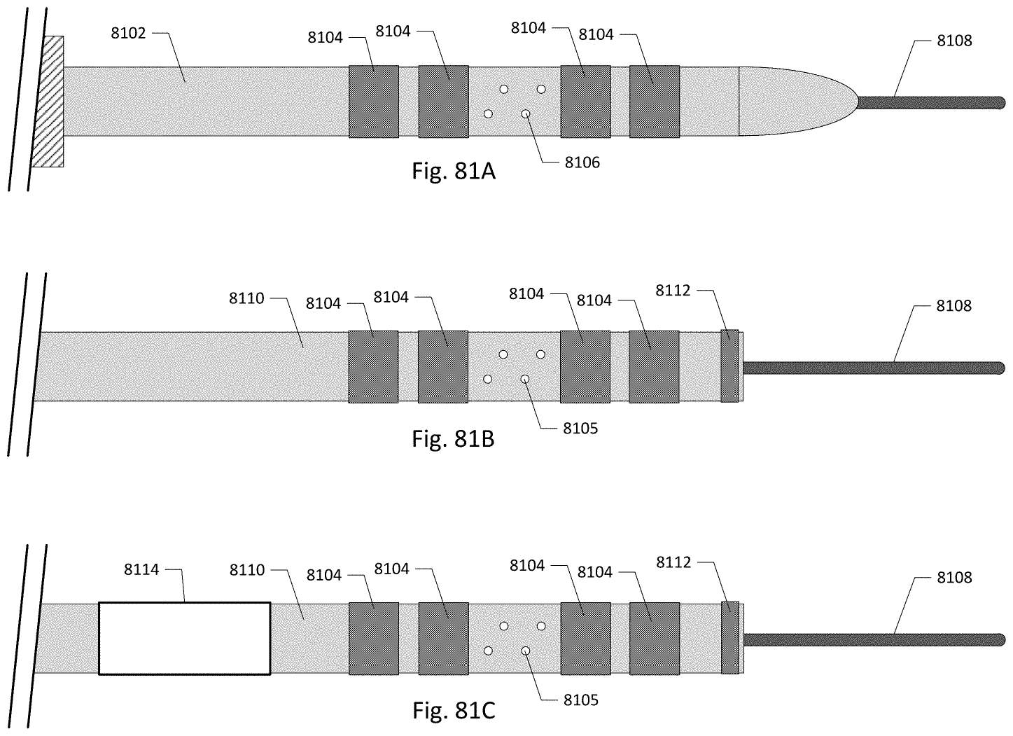

FIGS. 81A-C shows examples of vascular navigation systems in use with a pressure wire. FIG. 81A shows navigation device 8102 in the form of a stylet, including electrodes 8104 and fluid openings 8106. Also shown is pressure wire 8108. The pressure wire may be incorporated into the navigation device or used through, or next to, the navigation device. The pressure wire may be in communication with the controller of the navigation system.

FIG. 81B shows navigation device 8110 in the form of a catheter. The embodiment shown includes additional electrode, such as ECG electrode, 8112. In this embodiment, pressure wire 8108 is used through the inner lumen of the catheter.

FIG. 81C shows navigation device 8110 in the form of a catheter which includes balloon 8114. The balloon may be used for angioplasty of other purposes. The balloon may be proximal to, distal to, between or in any other location with respect to the electrodes. More than one pair of sensors and fluid openings may be present, as shown in FIGS. 80A-E. In any of the embodiments, The pressure wire may be incorporated into the navigation device or used through, or next to, the navigation device. The pressure wire may be in communication with the controller of the navigation system.

The infusion fluid in embodiments which use conductance/resistance sensors/electrodes may be of a higher or lower salinity (i.e. higher or lower conductivity) than blood. For example, the infusion fluid may be distilled water, Dextrose 5% in Water (D5 W), etc.

The controller may also integrate with other systems, such as electronic medical systems, electronic health systems etc. The integration may be wired or wireless and may be local or remote. The integration may be via “EMR sniffers”.

As mentioned herein, vascular location using fluid injection can also be determined through conductivity sensing, for example, where the sensor(s) include one or more electrodes. In these embodiments, the sensor(s), or electrode(s), measure the conductivity (or impedance) of the blood/medium mix, where the medium has a conductivity which is different than that of blood. For example the medium infused into the vessel may have a higher or lower conductivity than that of blood.

Salts are conductive, thus the salinity of the blood/medium mixture may be determined by the voltage drop across a pair of electrodes. For example, if a fluid that is less conductive than blood (such as a lower salinity saline solution (for example, 0.45% NaCl saline) or a solution without salt, such as H2O or a dextrose solution) is the medium which is introduced into the blood stream, a conductivity sensor can measure the presence of that fluid in the blood stream by measuring the conductivity of the blood/medium mixture within the blood flow. The measurement of conductivity over time/device position within the vessel can be used to determine laminar flow, turbulent flow, flow direction, etc. at the device tip or at the sensor(s) location. As a reference, the salinity of blood is around 0.9%.

Alternatively, a medium with a higher conductivity than that of blood may be used. For example, a 3% NaCl solution may be used to detect the fluid flow characteristics. In addition, a hypertonic solutions may increase the signal strength of measured ECG signals. All of the configurations described herein using temperature sensors or other sensors may alternatively use conductivity or impedance sensors. In some embodiments, the electrodes that serve as sensors may detect an ECG signal without an exterior (i.e. skin) ground electrode.

Embodiments of the vascular navigation device may include the ability to measure cardiac output or cardiac flow rate. The parameter vs. time/location curve may be analyzed by the controller to determine cardiac output in addition to vascular location, either simultaneously, or at separate times. Cardiac output may also be used to help establish the location of the vascular navigation device within the vasculature.

Embodiments of the vascular navigation device may include the ability to measure blood flow rate in other areas of the body/vasculature.

Several embodiments have been disclosed herein. It will be understood that any of the features of any of the embodiments may be combined with any embodiment.

Some embodiments of the vascular access or vascular navigation device may be used in other applications. For example, the controller of the device may be equipped with logic to navigate, identify, and assess the health of various vascular or other anatomies. For example, some embodiments may be configured to identify the location of valves within the peripheral vascular (for example, venous) system. Valve location may be identified based on the flow characteristics near and within a valve. Valve health may be assessed based on flow characteristics near and within a valve. Valve function may be assessed based on flow characteristics near and within a valve. Valve closure may be assessed based on flow characteristics near and within a valve. Vascular flow characteristics may be used by the system to navigate near to, within, and/or past valves. Some embodiments of the vascular navigation device may be used in conjunction with treatment procedures. For example, the system may be used to aid in placement of valve prosthetics, valve repair etc. The system may be used to assess the success of such procedures, based on flow characteristics, placement location etc. The system may also be used to navigate to vessel stenting locations, and to assess the function of a vessel before and after a procedure. The system may be used to assess the function and/or location and/or health of a prosthetic (stent, valve etc.) before and after its placement.

In some embodiments, the system may be used to diagnose a stenosis, blockage, narrowings or disease of a blood vessel based on flow characteristics. The system may be used to classify a stenosis, blockage, narrowings or disease of a blood vessel based on flow characteristics. The system may be used to identify the location and quantity of spinal fluid leak.

In some embodiments of the system, the vascular system is accessed peripherally, via a leg, arm, groin, etc.

Some embodiments of the system may be used to diagnose other diseases or health based on flow characteristics of vessels or other organs (such as the bladder, lungs, etc.)

Some embodiments of the system may be used to assess health of, and navigate through, other vessels such as those in the brain. For example, the system may be used to identify, navigate to and assess the health of, aneurysms, blockages, narrowings, stenosis with the brain and elsewhere in the body.

Embodiments of the system may be used for any interventional radiology procedure including Angiography, Arteriovenous Malformations (AVM), Balloon Angioplasty, Biliary Drainage and Stenting, Bleeding Internally, Central Venous Access, Chemoembolization, Embolization, Gastrostomy Tube, Hemodialysis Access Maintenance, High Blood Pressure, Infection and Abscess Drainage, Needle Biopsy, Radiofrequency Ablation, Stent, Stent Graft, Thrombolysis, TIPS (Transjugular Intrahepatic Portosystemic Shunt), Urinary Tract Obstruction, Uterine Artery Embolization, Uterine Fibroid Embolization, Varicocele Embolization, Varicose Vein Treatment, Vena Cava Filter, Vertebroplasty, Deep Vein Thrombosis, etc.

Some embodiments of the system may be used to identify blood flow direction, speed, flow characteristics, etc. This may be useful not only for navigation of the venous system, but also in assessing venous or arterial flow conditions that are useful for identifying heart disease, chronic venous disorder, venous outflow obstructions, etc.

Some embodiments of the system may be used to identify the change in flow characteristics of the blood as it responds to drugs such blood thinners (heparin, etc.) acutely or over time. For example, blood thinness, viscosity, or other properties may be assessed based on the flow characteristics.

Some embodiments of the multi sensor technology may also be included in a permanent implant within the body rather than used as a temporary device. It may be used to measure the performance or health of the cardiovascular system over time, measure post intervention performance over time, etc. This type of intervention may be surgical only, such as when used in a bypass procedure, and may also include monitoring the results and/or performance, and/or success of interventions such as mechanical valves, stents, balloons, etc. It may also be used for the assessment of the need for interventions.

In any of the embodiments disclosed herein, in addition to or instead of measuring temperature of a fluid bolus or stream that is injected, the system may measure the electrical conductivity of a bolus or stream of fluid. As a stream or bolus of fluid fluctuates with various flow conditions and directions, variation in electrical conductivity can be detected. Additionally, fluid may be injected to optimize the electrical conductivity. For example, fluid containing one or more salts may be used to make the fluid more electrically conductive, or, for example, fluid which is less conductive than blood may be used, such as distilled water, or dextrose water.

This technology may also be used outside of the body on the surface of the skin in proximity to one or more veins. This may be done on the skin or just under the skin, across the skin or within the skin. For example, temperature sensors may be placed in several locations on top of the skin or vein. A heating or cooling event may be administered intravascularly to detect blockages, flow, or navigation requirements. Conversely, the heating and or cooling event may happen externally to the skin while the system senses the temperature intravascularly. Alternatively, pressure, or electrical conductivity may be used. Some embodiments may also detect flow characteristics, diagnose venous or arterial disease, challenges, and obstructions, in either acute or chronic events. Embodiments of the device on the surface of the body or vein may be a temporary assessment tool, or may be a more permanently worn biosensor such as a watch, ring, wristband, necklace, earing, contact lens, etc.

From the foregoing, it will be appreciated that specific embodiments of the invention have been described herein for purposes of illustration, but that various modifications may be made without deviating from the spirit and scope of the various embodiments of the invention. For example, several embodiments may include various suitable combinations of components, devices and/or systems from any of the embodiments described herein. Further, while various advantages associated with certain embodiments of the invention have been described above in the context of those embodiments, other embodiments may also exhibit such advantages, and not all embodiments need necessarily exhibit such advantages to fall within the scope of the invention.

What is claimed is:

1. A location detection system, comprising:
an elongate body having a lumen, wherein the elongate body is sized for introduction and translation through a catheter lumen;
one or more openings located at or in proximity to a distal end of the elongate body;
a sensor positioned at or in proximity to the distal end of the elongate body, wherein the sensor is configured to measure at least one parameter of a mixture of a first fluid and a second fluid after the first fluid is emitted from the one or more openings and into the second fluid when the distal end of the elongate body is advanced beyond a distal opening of the catheter lumen; and
a controller in communication with the sensor, wherein the controller is configured to receive a signal indicative of the at least one parameter of the mixture and is further configured to obtain a position of the sensor within a body of a subject based upon the signal.

2. The system of claim 1 wherein the distal end of the elongate body is positionable at a distance of 5 mm to 40 mm beyond the distal opening of the catheter lumen.

3. The system of claim 1 wherein the distal end of the elongate body is positionable at a distance of 10 mm to 20 mm beyond the distal opening of the catheter lumen.

4. The system of claim 1 further comprising a proximal sensor positioned along the elongate body proximal to the sensor.

5. The system of claim 4 wherein the proximal sensor is positionable at a distance of 0 mm to 5 mm beyond the distal opening of the catheter lumen.

6. The system of claim 4 wherein the one or more openings are located along the elongate body between the sensor and the proximal sensor.

7. The system of claim 1 further comprising one or more leads coupled to the sensor, wherein the one or more leads are embedded within an outer wall of the elongate body.

8. The system of claim 7 wherein the one or more leads are embedded in a coiled configuration within the outer wall.

9. The system of claim 7 wherein the sensor comprises at least one pair of electrodes configured as a band.

10. The system of claim 7 wherein the one or more leads are coupled to the sensor through at least one opening defined within the outer wall.

11. The system of claim 7 wherein the sensor comprises at least one electrode which is crimped upon the elongate body such that the at least one electrode is electrically coupled to the one or more leads.

12. The system of claim 7 wherein the sensor comprises at least one electrode which defines a flat section.

13. The system of claim 7 wherein the sensor comprises at least one electrode having a piercing mechanism.

14. The system of claim 7 further comprising a proximal sensor positioned along the elongate body proximal to the sensor, wherein the proximal sensor comprises at least one proximal electrode.

15. The system of claim 7 wherein the one or more openings are located along the elongate body between the one or more leads embedded in the coiled configuration.

16. The system of claim 1 further comprising at least one electrode positioned at or in proximity to the distal end of the elongate body.

17. The system of claim 16 wherein the controller is configured to sense electrocardiogram signals from the body via the at least one electrode.

18. The system of claim 17 wherein the controller is further configured to determine the position of the sensor within the body of the subject based upon the electrocardiogram signals.

19. The system of claim 1 wherein the controller is further configured to determine the position of the sensor within the body of the subject based upon an electrocardiogram signal.

20. The system of claim 1 further comprising a disposable infusion pump in fluid communication with the elongate body.

21. The system of claim 1 further comprising one or more additional sensors in communication with the controller, wherein the one or more additional sensors are positioned along a catheter defining the catheter lumen.

22. A method of determining a location within a body of a subject, comprising:
introducing a first fluid through a lumen defined within an elongate body and through one or more openings located at or in proximity to a distal end of the elongate body after the distal end of the elongate body has been advanced beyond a distal opening of a catheter lumen;
measuring a signal via a sensor of at least one parameter of a mixture of the first fluid and a second fluid after the first fluid is emitted from the one or more openings and into the second fluid; and
determining via a controller in communication with the sensor a position of the sensor within the body of the subject based upon the signal.

23. The method of claim 22 further comprising introducing the elongate body intravascularly within the body of the subject prior to introducing the first fluid.

24. The method of claim 22 wherein the distal end of the elongate body has been positioned at a distance of 5 mm to 40 mm beyond the distal opening of the catheter lumen.

25. The method of claim 22 wherein the distal end of the elongate body has been positioned at a distance of 10 mm to 20 mm beyond the distal opening of the catheter lumen.

26. The method of claim 22 wherein measuring the signal further comprises measuring a second signal via a proximal sensor positioned along the elongate body proximal to the sensor.

27. The method of claim 26 wherein the proximal sensor has been positioned at a distance of 0 mm to 5 mm beyond the distal opening of the catheter lumen.

28. The method of claim 26 wherein the one or more openings are located along the elongate body between the sensor and the proximal sensor.

29. The method of claim 22 wherein measuring the signal further comprises transmitting the signal through one or more leads which are coupled to the sensor, wherein the one or more leads are embedded within an outer wall of the elongate body.

30. The method of claim 29 wherein the one or more leads are embedded in a coiled configuration within the outer wall.

31. The method of claim 29 wherein the sensor comprises at least one pair of electrodes configured as a band.

32. The method of claim 29 wherein the one or more leads are coupled to the sensor through at least one opening defined within the outer wall.

33. The method of claim 29 wherein the sensor comprises at least one electrode which is crimped upon the elongate body such that the at least one electrode is electrically coupled to the one or more leads.

34. The method of claim 29 wherein the sensor comprises at least one electrode which defines a flat section.

35. The method of claim 29 wherein the sensor comprises at least one electrode having a piercing mechanism.

36. The method of claim 29 further comprising a proximal sensor positioned along the elongate body proximal to the sensor, wherein the proximal sensor comprises at least one proximal electrode.

37. The method of claim 29 wherein the one or more openings are located along the elongate body between the one or more leads embedded in the coiled configuration.

38. The method of claim 22 further comprising measuring a second signal via at least one electrode positioned at or in proximity to the distal end of the elongate body.

39. The method of claim 37 wherein measuring the second signal comprises measuring electrocardiogram signals from the body via the at least one electrode.

40. The method of claim 38 further comprising determining via the controller the position of the sensor within the body of the subject based upon the electrocardiogram signals.

41. The method of claim 22 further comprising determining via the controller the position of the sensor within the body of the subject based upon an electrocardiogram signal.

42. The method of claim 22 wherein introducing the first fluid comprises introducing the first fluid via a disposable infusion pump in fluid communication with the elongate body.

43. The method of claim 22 further comprising receiving additional signals from one or more additional sensors via the controller, wherein the one or more additional sensors are positioned along a catheter defining the catheter lumen.
